(19) United States
(12) Patent Application Publication
(10) Pub. No.: US 2007/0224648 A1
(43) Pub. Date: Sep. 27, 2007

(54) Methods and compositions for the diagnosis of treatment of type 2 diabetes

(75) Inventors: Roger D. Cox, Oxfordshire (GB); Frances Ashcroft, Oxford (GB); Helen Freeman, Boston, MA (US); Kenju Shimomura, Oxford (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: The Chancellor, Masters and Scholars of the University of Oxford, London (GB)

(21) Appl. No.: 11/523,479

(22) Filed: Sep. 19, 2006

Related U.S. Application Data
(60) Provisional application No. 60/718,905, filed on Sep. 20, 2005.

(51) Int. Cl. G01N 33/567; A61K 31/155; C12N 5/06; C12N 1/18

(52) U.S. Cl. 435/7.2; 435/325; 435/455; 514/635; 435/254.2

(57) ABSTRACT

Methods and compositions for the modulation of insulin secretion.

The present application claims the benefit of priority of U.S. Provisional Patent Application No. 60/718,905, which was filed Sep. 20, 2005 and is incorporated herein by reference in its entirety.

### FIELD OF THE INVENTION

The present invention relates to methods and compositions for the treatment of diabetes.

### BACKGROUND OF THE RELATED ART

Type 2 diabetes (also called non-insulin dependent diabetes mellitus (NIDDM) or adult onset diabetes) is a serious metabolic disease that is reaching epidemic proportions in Western societies and is predicted to affect 300 million people worldwide by 2025 (King et al., Diabetes Care, 21:1414-1431 (1998)). Over 90% of diabetes is of the type 2 kind. An annual expenditure of $100 billion is attributed to the disease in the United States alone. It is the third leading cause of death each year. Prolonged untreated diabetes leads to heart disease, stroke, kidney disease, blindness, and loss of limbs from advanced peripheral vascular disease. Further, impaired glucose tolerance, which precedes diabetes and is a risk factor for the disease, currently affects a further 200 million worldwide.

Type 2 diabetes is characterized by elevation of the blood glucose concentration, usually presents in middle age, and is exacerbated by age and obesity. It is associated with both impaired insulin secretion and insulin action but it is now recognised that β-cell dysfunction is a key element in the development of the disease (Bell et al., Nature 414:788-791 (2001); Kahn, Diabetologia 46:3-19 (2003)); (Ashcroft et al., Hum Mol Genet 13(1):R21-R31 (2004)).

Insulin release from pancreatic β-cells is stimulated by increased β-cell uptake and metabolism of glucose. The consequent changes in the intracellular concentrations of adenine nucleotides cause closure of ATP-sensitive K⁺ (K_{ATP}) channels in the β-cell plasma membrane. In turn, this leads to membrane depolarisation, opening of voltage-gated Ca²⁺ channels, Ca^{2 +} influx, fusion of insulin secretory vesicles with the plasma membrane and insulin secretion. Normally, insulin secretion in response to elevated plasma glucose is biphasic. Intracellular messengers controlling K_{ATP} channel-dependent first phase insulin secretion also regulate second phase insulin secretion. However, additional K_{ATP} channel-independent messengers are also involved [Straub et al., Diabetes Metab Res Rev 18:451-463 (2002)]. There is evidence that loss of first phase insulin secretion leads to postprandial hyperglycaemia and is common both in patients with Type 2 diabetes and individuals with impaired glucose tolerance [Del Prato, Diabetologia 46 (Suppl. 1):M2-M8 (2003)].

Defects in β-cell function are found in monogenic diabetes, such as maturity-onset diabetes of the young [Bell et al., Nature 414:788-791 (2001)] and permanent neonatal diabetes. It is also apparent that abnormalities in insulin secretion and β-cell function contribute to the onset and development of polygenic Type 2 diabetes [Bell et al., Nature 414:788-791 (2001)]. In Type 2 diabetes there is gradual progression from normal glucose tolerance, to impaired glucose tolerance and subsequently overt diabetes. This is associated with a progressive decline in β-cell function and reduced insulin secretion. Insulin resistance may enhance the risk of diabetes by placing an increased demand upon the β-cell, but by itself does not result in diabetes. The genetic defects that produce inappropriate homeostatic control in Type 2 diabetes are poorly understood [Bell et al., Nature 414:788-791 (2001); Florez et al., Annu Rev Genomics Hum Genet 4:257-291 (2003)].

Animal models of glucose intolerance may provide valuable information about glucose homeostasis that can ultimately be applied to human diabetes. The C57BL/6J mouse exhibits defects in glucose tolerance that are independent of obesity [Kaku et al., Diabetes 37:707-713 (1988); Kooptiwut et al., Endocrinology 143:2085-2092 (2002)]. Feeding C57BL/6J mice with high-fat diets results in insulin resistance, increased fasting plasma glucose levels and diabetes [Surwit et al., Diabetes 37:1163-1167 (1988); Surwit et al., Diabetes 40:82-87 (1991); Burcelin et al., Am J Physiol Endocrinol Metab 282:834-842 (2002); Fueger et al., Diabetes 53:306-314 (2004)]. On a high fat diet these mice also show higher weight gain per calorie [Surwit et al., Metabolism 44:645-651 (1995)], higher weight gain when the same amount of calories are given as fat [Petro et al., Metabolism 43:454-457 (2004)], and higher fat and lower protein and water body composition [Black et al., Metabolism 47:1354-1359 (1998)] than A/J mice. Insulin action was also reduced by between 32% and 60% in C57BL/6J mice after 9 months on a high fat diet [Burcelin et al., Am J Physiol Endocrinol Metab 282:834-842 (2002)]. Interestingly, however, C57BL/6J mice are actually more insulin sensitive than AKR/J mice [Rossmeisl et al., Diabetes 52:1958-1966 (2003)], and DBA/2 or 129X1 mice [Goren et al., Endocrinology 145:3307-3323 (2004)].

On normal diets, C57BL/6J mice appear to have normal free-fed plasma insulin levels, but post-prandial first phase insulin release is impaired when compared to glucose-tolerant control strains, including C3H [Kaku et al., Diabetes 37:707-713 (1988); Kayo et al., Comp Med 50:296-302 (2000)]. Second phase insulin release is also impaired, in comparison with AKR/J [Rossmeisl et al., Diabetes 52:1948-1966 (2003)] and DBA/2 [Kooptiwut et al., Endocrinology 143:2085-2092 (2002)] mice, but is not significantly different to C3H mice (at least at 8 weeks of age; [Kaku et al., Diabetes 37:707-713 (1988)]). A defect in first phase insulin secretion also appears to be present in C57BL/6J mice fed a high fat diet [Ahren, Am J Physiol Endocrinol Metab 283:E738-E744 (2002)], and is accompanied by an unchanged insulin response and delayed glucose clearance over the first 4 weeks of a high fat diet [Reimer et al., Diabetes 51:S138-S143 (2002)]. Recent physiological [Burcelin et al., Am J Physiol Endocrinol Metab 282:834-842 (2002)] and genetic studies [Surwit et al., Diabetes 40:82-87 (1991); Stoehr et al., Diabetes 49:1946-1954 (2000)] also suggest that insulin and plasma glucose levels are poorly correlated. Classification of C57BL/6J mice on a high fat diet into lean non-diabetic (12%), lean diabetic (12%), obese diabetic (about 50%), and intermediates, has allowed a microarray transcript profiling comparison to be made and showed striking differences in gene expression between the groups [Fourmestraux et al., J Biol Chem, published on line 10.1074/jbc.M408014200 (2004)]. These data suggest a differential metabolic adaptation, not wholly under genetic control, that contributes to the observed phenotypic diversity in this strain [Fourmestraux et al., J Biol Chem, published on line 10.1074/jbc.M408014200 (2004)].

In summary C57BL/6J mice represent an important model of diet-induced diabetes which also exhibits defects in glucose tolerance on a normal diet. This model develops insulin resistance on a high fat diet and appears to have a complex insulin secretion deficit under both normal and high fat diets. It can be used to assess diabetes targets for treatment. Being a complex multifactorial disorder, much remains to be understood about type 2 diabetes and methods of treatment thereof. The present invention provides guidance as to methods of determining susceptibility to this disorder as well as methods of treatment thereof.

### SUMMARY OF THE INVENTION

The present invention is directed to methods of identifying an agent that modulates glucose-stimulated insulin secretion in an animal, the method comprising the steps of: (i) contacting an agent to a wild-type nicotinamide nucleotide transhydrogenase (NNT) polypeptide comprising at least 20 contiguous amino acids of SEQ ID NO:2 or 4; (ii) selecting an agent that binds to the polypeptide or modulates the expression or activity of the polypeptide, or its targeting to the mitochondria and (iii) determining the effect of the selected agent on glucose-stimulated insulin secretion, thereby identifying an agent that modulates glucose-stimulated insulin secretion in an animal.

Other methods involve identifying an agent that modulates glucose-stimulated insulin secretion in an animal or cell, the method comprising the steps of: (i) contacting an agent with an NNT polypeptide; and (ii) selecting an agent that modulates the expression or activity of the polypeptide or its targeting to the mitochondria, thereby identifying an agent that modulates glucose-stimulated insulin secretion in an animal.

Step (ii) of these methods preferably comprises selecting an agent that modulates the expression of the polypeptide; alternatively, or in addition, step (ii) comprises selecting an agent that modulates the activity of the polypeptide; in yet a third alternative, step (ii) comprises selecting an agent that modulates the correct targeting of the polypeptide to the mitochondria.

In certain embodiments, the polypeptide is a mutation or polymorphism in the polypeptide of SEQ ID NO: 2 or SEQ ID NO:4. In other embodiments, the polypeptide is a wild-type NNT as shown in SEQ ID NO:2 or SEQ ID NO:4. The wild-type polypeptide is encoded by a nucleic acid sequence of SEQ ID NO: 1 or 3. Human NNT and its nucleic acid sequence is known to those of skill in the art and it is given provided at UniGene Hs.482043 (Homo sapiens NNT) and Entrez Gene ID No. 23530. A representative sequence is given at GenBank Accession No. NM_{—}012343 (SEQ ID NO:1 for nucleic acid sequence and SEQ ID NO:2 for protein sequence) and also at GenBank Accession No. NM_{—}182977 (SEQ ID NO:3 for nucleic acid sequence and SEQ ID NO:4 for protein sequence). These are merely representative sequences and those of skill in the art will be able to identify other NNT sequences in publicly available databases as well as to identify variations thereof that will be useful in the context of the present invention.

In some screening methods, the polypeptide is linked to a solid support. In other screening methods the candidate agent is linked to a solid support.

In some embodiments, the agent is selected by identifying an agent that specifically binds to the polypeptide.

Some screening methods are cell-based screening methods in which the NNT is provided in a cell that expresses the NNT. In other screening methods, isolated organelles (especially mitochrondria) from such cells are used.

The screening method may use any functional output, e.g., the activity of the polypeptide is determined by a step comprising measuring a change in calcium flux in said cell, and/or the activity of the polypeptide is determined by a step comprising measuring a change in membrane potential of a cell, and/or the activity of the polypeptide is determined by a step comprising measuring a change in insulin secretion by the cell; and/or the activity of the polypeptide is determined by the step comprising measuring a change in glucose-stimulated insulin secretion by the cell. As noted above, the cell may be an insulin-secreting cell or an intracellular organelle of such a cell.

In specific screening methods the method further comprising administering the agent to a diabetic animal and testing the animal for increased glucose-stimulated insulin secretion. The animal may have a mutant NNT activity that causes an impaired β-cell function and said agent improves said impaired β-cell function.

Also provided are methods of inducing glucose-stimulated insulin production in an animal, the method comprising administering a therapeutically effective amount of the agent selected in the above methods. Preferably, the animal is a human. The method may further comprise administering metformin to the animal.

Other embodiments provide methods of determining whether a patient is susceptible to developing polygenic type 2 diabetes comprising determining the presence of a mutation in the NNT gene of said patient, wherein a mutation in the NNT gene of said patient is indicative of a susceptibility to polygenic type 2 diabetes.

Also encompassed by the invention is a recombinant cell transfected with the polynucleotide that encodes an NNT polypeptide wherein said cell is an insulin secreting cell. The cell may express a mutant NNT protein that contains a polymorphism or other mutation or a wild-type NNT protein. While the cell may be a pancreatic islet cell, it is contemplated that the cell may be any isolated cell type that has been transfected to encode an NNT protein or naturally encodes Nnt.

Also provided are methods for treating diabetes in a patient, comprising: administering to said patient an effective amount of an agent that augments, potentiates or otherwise increases the activity of NNT in said patient. The patient may have a mutant NNT for example one that has a polymorphism and said agent is administered to overcome the effects of said mutation. The effects said mutation comprise a reduced insulin secretion, a glucose-dependent increase in [Ca²⁺]i in the β-cells of said patient, an increased glucose intolerance, impaired glucose dependent β-cell electrical activity, a decrease in β-cell ATP production, or enhance K_{ATP} channel activity. The method is one which comprises administering a composition that comprises a wild-type NNT to said patient. Preferably, the agent enhances NNT expression and/or activity. In specific embodiments, the patient has Type 2 Diabetes. In other embodiments, the patient has impaired the β-cell function and said administration improves said β-cell function. In still other embodiments, the administering results in increased islet blood flow, increased pancreatic β-cell perfusion, reduced insulin resistance in skeletal muscles, increased insulin-mediated glucose disposal, or increased insulin-mediated glucose uptake by skeletal muscles. In some embodiments, these methods may further involve administration to the patient a therapeutically effective amount of a sulfonylurea agent or a biguanide agent. Such agents and therapeutic regimens for determining the dosage and efficacy of such agents are well known to those of skill in the art. Exemplary such agents available in the art include, but are not limited to biguanide compounds selected from the group consisting of metformin, phenformin and buformin. In specific embodiments, the patient is treated with a therapeutically effective amount of metformin.

Also contemplated herein are methods of screening for an agent that modulates NNT activity comprising culturing a cell that has been transfected with the polynucleotide that encodes an NNT polypeptide wherein the cell is an insulin secreting cell and determining the mitochondrial calcium levels in the cell being cultured in prior to and after addition of a candidate agent that modulates NNT activity wherein an alteration in the mitochondrial calcium level of the cell in the presence of the candidate agent identifies the agent as an NNT modulator. Such screening methods may further comprise elevating the reactive oxygen species (ROS) of the cell in culture prior to addition of the candidate agent. Methods of elevating ROS may involve addition of glucose, menadione, hydrogen peroxide or a combination thereof to the cells.

Other embodiments contemplate methods of screening for an agent that modulates NNT activity comprising culturing a yeast cell transformed or transfected with the polynucleotide that encodes an NNT polypeptide and determining the membrane potential and/or mitochondrial pH in the yeast cell being cultured prior to and after addition of a candidate agent that modulates NNT activity wherein an alteration in the membrane potential and/or mitochondrial pH of the cell in the presence of the candidate agent identifies the agent as an NNT modulator.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1: Male mice at age 12 weeks: (FIG. 1A) Intraperitoneal glucose tolerance test (IPGTT) and, (FIG. 1B) Area under IPGTT curve (AUC) in units of min mmol/l, mean value profiles of 4 inbred strains. A, DBA/2 open circles (n=10), BALB/C open triangle (n=10), C3H/HeH open squares (n=10), C57BL/6J filled triangle (n=10). B, from left to right BALB/C (n=10), C3H/HeH (n=10), C57BL/6J (n=10), DBA/2 (n=10), F1 (B/C, n=53) male offspring of a cross between C57BL/6 mothers and C3H/HeH fathers, F1 (C/B, n=72) are F1 male offspring of a cross between C3H/HeH mothers and C57BL/6 fathers. Y-error bars represent standard errors of the mean.

FIG. 2: interval map of T60 glucose on chromosome 13 (FIG. 2A), 11 (FIG. 2B) and 9 (FIG. 3C). Sugg. (L&K) and Sig. (L&K), are widely accepted thresholds for declaring suggestive and significant linkage [Lander et al., Nat Genet 11:241-247 (1995)]. Sig. (perm) is the empirical false discovery threshold for declaring significant T60 glucose linkage in the current dataset and was derived from analysis of 1000 permutations of the original dataset.

FIG. 3: Plasma glucose dynamics of 12 week old free fed male mice in an insulin tolerance test. Free fed plasma glucose is significantly higher in C57BL/6J mice (filled triangles, p=0.001). Plasma glucose clearance is significantly more rapid in C57BL/6 than C3H/HeH (filled squares) in the first 10 minutes of the test (p=0.03). No significant difference in plasma glucose clearance rate/or level at all other time points. Y-error bars represent standard deviation values.

FIG. 4: Insulin secretion in response to glucose is impaired in isolated pancreatic islets from C57BL/6J compared to C3H/HeH mice. White bars C57BL/6J pancreatic islets; Black bars, C3H/HeH pancreatic islets. The numbers above the bars indicate the numbers of replicate wells (each well contained 10 islets). Y-error bars represent standard errors of the mean.

FIG. 5: (FIG. 5A) Representative changes in intracellular Ca2+, measured by Fura-2 fluorescence, in response to glucose and tolbutamide in single β-cells isolated from C57BL/6J (top), and C3H/HeH (bottom) mice. The bars indicate when glucose (2, 5, 10 mmol/l) and tolbutamide (500 μmol/l) were applied. (FIG. 5B) Mean change in fluorescence ratio in response glucose or tolbutamide (as indicated) in C57BL/6J (black bars, n=7) and C3H/HeH (grey bars, n=11) β-cells. *, p<0.05 (t-test). Y-error bars represent standard errors of the mean.

FIG. 6: (FIG. 6A) Representative whole-cell perforated patch recordings from a C3H/HeH (top) and a C57BL/6J (bottom) mouse β-cell in response to ±20 mV pulses from −70 mV. Glucose (5 mmol/l) or tolbutamide (200 pmol/l) were applied to the external solution as indicated. The dotted line shows the zero-current level. (FIG. 6B) Mean glucose concentration-response relationships for whole-cell K_{ATP} currents from C57BL/6J (circles, n=13) or C3H/HeH (squares, n=6) β-cells. Current (I) is expressed as a fraction of the mean (I0) of that obtained in control solution before and after exposure to glucose. The lines are the best fit of the Hill equation data to the data. For C57BL/6J mice, IC50=24.7±2.9 μmol/l, h=1.15±0.16 (n=13). For C3H/HeH mice, IC50=5.7±0.2 μmol/l, h=1.16±0.04 (n=6). (FIG. 6C) Single-channel currents recorded at −60 mV from inside-out patches excised from a C57BL/6J or CH3/HeH β-cell. ATP (10 μmol/l) was applied to the intracellular solution as indicated. The dotted line indicates the zero-current level. (FIG. 6D) Mean ATP concentration-response relationship for K_{ATP} channels from C57BL/6J β-cells. Channel activity (NPo(ATP)) is expressed as a fraction of the mean (NPo) of that obtained in control solution before and after exposure to ATP. The line is the best fit of the Hill equation to the data: IC50=5.7±0.5 pmol/l, h=0.89±0.06 (n=5). Y-error bars represent standard errors of the mean.

FIG. 7: Quantitative RT-PCR assessment of Nnt gene expression in liver (first four pairs of bars, n=4 C3H/HeH and n=4 C57BL/6J, fasted (p<0.01) and free fed (p<0.01) mice) and isolated pancreatic islets (approximately 300 islets from each strain pooled to make RNA, last 2 pairs of bars) of 12 week old male mice. C3H/HeH (light grey bars) than C57BL/6 (black bars). RQ is relative quantitation statistic representing the Nnt/GAPDH expression ratio. RRQ is the relative RQ statistic expressing the ratio of the RQ statistic for the higher expressing strain (C3H/HeH) relative to that for the lower expressing strain (C57BL/6). Y-error bars represent standard deviation values.

FIG. 8: Mechanism of insulin secretion from pancreatic β-cells.

FIG. 9: Effect of Nnt knockdown on insulin release from MIN6 cells. FIG. 9A. Comparative levels of Gck (top) and Nnt (bottom) gene expression using quantitative PCR in MIN6 cells transfected with nonsense siRNA (ns control, grey bar), Nnt siRNA (white bar) and Gck siRNA (black bar). The data are representative of 3 separate experiments. FIG. 9B. Western blots of C3H/HeH (C3H) or C57BL/6J (B6) mouse islets (left two lanes) and of MIN6 cells (right lanes) transfected with nonsense siRNA (top), Nnt siRNA (middle) or Gck siRNA (bottom). NS, nonsense-transfected MIN6 cells for comparison. Numbered lanes, number of MIN6 repeats of transfected cells. Each blot was probed with three different antibodies (Ab), as indicated on the left, against Nnt, Gck and actin (loading control, not shown). The data are representative of 7 separate experiments. FIG. 9C. Insulin secretion in response to glucose (0, 2, 10 mM) or tolbutamide (200 μM) from MIN6 cells transfected with nonsense siRNA (white bars, n=6) or with Nnt siRNA (black bars, n=6). The data are representative of 3 separate experiments.

FIG. 10: Effect of Nnt knockdown on Ca2+ transients in MIN6 cells. FIG. 10A. Representative changes in intracellular Ca2+, measured by Fura-2 fluorescence, in response to glucose and tolbutamide in single MIN6 cells. Bars indicate when glucose (2, 5, 10 mM glucose) or tolbutamide (200 μM, Tol) were applied. Each colour represents a different cell. FIG. 10B. Representative changes in [Ca2+]i, measured by Fura-2 fluorescence, in response to glucose and tolbutamide in single MIN6 cells transfected with Cy3-conjugated siRNA duplexes for NntI or NntII. Bars indicate when glucose (2, 5, 10 mM glucose) or tolbutamide (200 μM, Tol) were applied. Each colour represents a different cell. FIG. 10C. Mean change in [Ca2+]i, in response to 2, 5 and 10 mM glucose, normalised to the resting [Ca2+]i, in 0 mM glucose for mock transfected (orange) MIN6 cells or MIN6 cells transfected with nonsense (blue), NntI (green), NntII (cyan) or Gck (pink) siRNA. Each data point is the mean of 15-21 cells. The data are representative of 5 separate experiments. FIG. 10D. Mean change in [Ca2+]i, in response to 200 μM tolbutamide for MIN6 cells mock transfected (orange) or transfected with nonsense (blue), NntI (green), NntII (cyan) or Gck (pink) siRNA.

FIG. 11: FIG. 11A. Alignment of Nnt amino acid sequence for various species across the regions in which mutations in Nnt were identified by screening the Harwell ENU-DNA archive. The mutated amino acids in exon 2 (N68K) and exon 14 (G745D) are indicated. FIG. 11B. Western blots of pancreatic islets isolated from homozygous N68K or G745D Nnt mutant mice. Each blot was probed with three different antibodies (Ab) as indicated on the left, against Nnt, Gck and actin (loading control, not shown). The data are representative of 4 separate experiments.

FIG. 12: FIGS. 11A,B Plasma glucose dynamics measured in response to an intraperitoneal glucose tolerance test (IPGTT) following overnight fasting for 12 week old male mice. The IPGTT was carried out over 2 hours. FIG. 12A, wild-type (circles n=9) heterozygous (squares, n=10) and homozygous (triangles, n=15) mice carrying the N68K mutation in Nnt. FIG. 11B, wild-type (circles, n=10), heterozygous (squares, n=16) and homozygous (triangles, n=13) mice carrying the G745D mutation in Nnt. Statistical significance of homozygote vs. wild-type and heterozygotes vs. wild-type is indicated, * p<0.05 and ** p<0.01 (student t-test). FIG. 12C-F. Plasma glucose (FIG. 12C, FIG. 12D) and insulin (FIG. 12E, FIG. 12F) dynamics measured in response to an IPGTT at 16 weeks for the same mice as in FIG. 12A, FIG. 12B. The IPGTT was carried out over 30 minutes. For key see FIG. 12A, FIG. 12B.

FIG. 13: FIG. 13A. Insulin secretion from pancreatic islets isolated from G745D Nnt mice in response to glucose (0, 2, 10 mM) or tolbutamide (tol, 200 μM). Islets were isolated from 2 mice of each type for these experiments. The data are representative of 2 separate experiments on G745D mice and 2 on N68K mice. Black bars are homozygous mutants, hashed bars are heterozygotes and white bars are wild-type littermates. FIG. 13B. ATP content of islets isolated from G745D Nnt mice in response to glucose (2 mM grey bars, 10 mM black bars). Each data point is the mean of 5 samples, each of 6 islets. Data is normalised to protein content. The data are representative of 2 separate experiments on G745D mice.

FIG. 14: Suggested mechanism for the effect of Nnt mutations on insulin secretion.

FIG. 15: FIGS. 15A,B. Plasma glucose dynamics measured in response to an intraperitoneal glucose tolerance test (IPGTT) following overnight fasting for 12 week old female mice. The IPGTT was carried out over 2 hours. FIG. 15A, wild-type (circles, n=9) heterozygous (squares, n=10) and homozygous (triangles, n=15) mice carrying the N68K mutation in Nnt. FIG. 15B, wild-type (circles, n=10), heterozygous (squares, n=16) and homozygous (triangles, n=13) mice carrying the G745D mutation in Nnt. Statistical significance of homozygote vs. wild-type and heterozygotes vs. wild-type is indicated, * p<0.05 and ** p<0.01 (student t-test). FIGS. 15C-F. Plasma glucose (FIG. 15C, FIG. 15D) and insulin (FIG. 15E, FIG. 15F) dynamics measured in response to an IPGTT at 16 weeks for the same mice as in FIG. 15A. The IPGTT was carried out over 30 minutes. For key see FIG. 15A.

FIG. 16: Mice carrying the N68K Nnt mutation show a similar ability to thermoregulate as control littermates. Rectal temperature profiles of male (FIG. 16A) and female (FIG. 16B) mice at room temperature (time zero) and then when housed at 4° C. for 6 hours. Wild type littermates (diamonds, n=5 of each sex) and mice heterozygous (triangles, 14 male and 20 female) or homozygous (squares, 9 male and 12 female) for the N68K mutation in Nnt.

FIG. 17: ROS measured using the fluorescent dye DCFA in single beta-cells isolated from wild-type mice (white bars) and homozygous NNT G745D mutant mice (black bars) at 0 mM and 20 mM glucose.

FIG. 18: ATP content of pancreatic islets isolated wild-type and homozygous NNT G745D mutant mice in response to glucose (2 mM grey bars, 10 mM black bars). Each data point is the mean of 5 samples, each of 6 islets. Data are normalised to protein content. The data are representative of 2 separate experiments on G745D mice.

FIG. 19: Glucose utilization in pancreatic islets isolated wild-type and homozygous NNT mutant mice (G745D). Each data point is the mean of 6 replicates each of 5 islets. Glucose utilization was measured as described in Ashcroft S J H, Weerasinghe L C C, Bassett J M, Randle P J: The pentose cycle and insulin release in mouse pancreatic islets. Biochem J (1972) 126, 525-532. Briefly, And these islets were placed in 20 microlitres of medium containing [3H]glucose and various concentrations of unlabelled glucose (as indicated). The tubes were placed in outer vials containing 0.5 ml of water. After 2-hours of incubation, metabolism was terminated by applying 5 microlitres of HCl. Then these samples were incubated for more than 18-24 hours to allow [3H]water in the incubation tube to equilibrate with the water in outer vial. Then, radioactivity of the water in the outer vial was measured. Control samples without islets were used to correct for background counts.

FIG. 20: Effect of H2O2 on glucose utilization in isolated wild-type islets. Each data point is the mean of 6 replicates each of 5 islets.

FIG. 21: Representative changes in [Ca2+]i, measured by Fura-2 fluorescence, in response to glucose in single MIN6 cells transfected with Cy3-conjugated siRNA duplexes for Nnt (A) or for Nnt plus UCP2 (B). Bars indicate when glucose (2, 5, 10 mM Glucose) or tolbutamide (200 μM, Tol) were applied. Each colour represents a different cell.

FIG. 22: Plasma glucose dynamics measured in response to an intraperitoneal glucose tolerance test (IPGTT) following overnight fasting in 12 week old male mice. The IPGTT was carried out over 2 hours.

FIG. 23: Plasma glucose and insulin dynamics in response to an intraperitoneal glucose tolerance test (IPGTT) following overnight fasting in 16 week old male mice, for the same mice as in FIG. 22. The IPGTT was carried out over 30 minutes.

FIG. 24. Insulin secretion from G745D pancreatic islets. Insulin secretion from islets isolated from wild-type or G745D mice in response to 1 hour incubation in 2 mM, 5 mM, 10 mM and 20 mM glucose. The data are the mean±SEM of six wells (5 islets/well) and are representative of 3 separate experiments.

FIG. 25. Insulin secretion from N68K pancreatic islets. Insulin secretion from islets isolated from wild-type or N68K mice in response to 1 hour incubation in 2 mM, 5 mM, 10 mM and 20 mM glucose. The data are the mean±SEM of six wells (5 islets/well) and are representative of a single experiment.

FIG. 26. Knockdown of Nnt alters the mitochondrial membrane potential in 20 mM glucose. MIN6 cells were transfected with nonsense siRNA (left) or Nnt siRNA (right). They were incubated for 3 hours in 2 or 20 mM glucose and then loaded with the dye JC1 and analysed by FACS. The X-axis shows green fluorescence (indicating a reduced mitochondrial membrane potential) and the Y-axis red fluorescence (indicating increased mitochondrial membrane potential). In 20 mM glucose, the average mitochondrial membrane potential is depolarized in MIN6 cells in which Nnt is knocked down. Data show results from ˜10,000 cells and are representative of 3 separate experiments.

FIG. 27. Loss of function mutations in Nnt alter the mitochondrial membrane potential of isolated beta-cells. Beta-cells isolated from wild-type (left) or Nnt-G745D pancreatic islets (right) were incubated for 3 hours in 2 or 20 mM glucose, as indicated, and then loaded with the dye JC1 and analysed by FACS. The X-axis shows green fluorescence (indicating a reduced mitochondrial membrane potential) and the Y-axis red fluorescence (indicating increased mitochondrial membrane potential). In 20 mM glucose, the average mitochondrial membrane potential is significantly depolarized in Nnt-G745D beta-cells. Data show results from ˜10,000 cells and are representative of 3 separate experiments.

FIG. 28. Incubation with 1 mM glutathione decreases ROS production in MIN6 cells transfected with Nnt siRNA. ROS measured using the fluorescent dye H₂DCFDA-SE in MIN6 cells transfected with nonsense or Nnt siRNA. Representative images of bright field and DCF fluorescence in MIN6 cells transfected with nonsense or Nnt siRNAs incubated in 20 mM glucose.

FIG. 29. Incubation with 1 mM glutathione decreases ROS production in MIN6 cells transfected with Nnt siRNA. ROS measured using the fluorescent dye H₂DCFDA-SE in MIN6 cells transfected with nonsense or Nnt siRNA, in response to 0 and 20 mM glucose, and 1 mM GSH, as indicated. Data are the mean of 10 cells and representative of 3 separate experiments.

FIG. 30. Glutathione partially restores the calcium response in Nnt siRNA transfected MIN6 cells. Mean change in [Ca²+]i, in response to glucose (2 mM, 20 mM) or tolbutamide (200 μM, Tolb) normalised to the resting [Ca²⁺]i in 0 mM glucose, for MIN6 cells transfected with nonsense siRNA (orange, control) or Nnt siRNA (blue, green). Cells were preincubated with (blue) or without (green, orange) 1 mM glutathione (GSH) for 4 hours. [Ca²⁺]i was measured by fura2 fluorescence. The data are representative of 2 separate experiments.

FIG. 31. Glutathione partially restores the insulin secretory response in Nnt siRNA transfected MIN6 cells. Insulin secretion in response to glucose (2 mM, 20 mM) or tolbutamide (200 μM) from MIN6 cells transfected with nonsense or Nnt siRNA, as indicated. The antioxidant glutathione (1 mM for 4 hours) was added as indicated. Menadione (1 mM) was added in some experiments as a positive control to increase ROS production. Data are representative of 2 separate experiments, each carried out in triplicate.

FIG. 32. Reduced glutathione is lower in Nnt-N68K mice pancreatic islets. Comparative levels of reduced glutathione (GSH) measured in islets isolated from wild-type and Nnt-N68K mice. Data are the mean of 6 replicates, each using 100-150 islets. The data are representative of 4 separate experiments.

FIG. 33. UCP2 expression is increased in Nnt-N68K mice pancreatic islets. Left, Western blots showing comparative levels of the uncoupling protein UCP2 measured in islets isolated from wild-type and Nnt-N68K mice, detected using an antibody against UCP2. Actin is shown as a positive control for gel loading. Data are the mean of 6 replicates, each using 120-150 islets. The data are representative of 4 separate experiments.

FIG. 34. Simultaneous knockdown of UCP2 and Nnt restores the wild-type response. Mean change in [Ca²⁺]i, in response to glucose (2 mM, 20 mM) or tolbutamide (200 μM, Tolb) normalised to the resting [Ca²⁺]i in 0 mM glucose, for MIN6 cells transfected with nonsense (black), Nnt (blue), UCP2 (magenta) or UCP2+Nnt (cyan) siRNAs. Each data point is the mean of 35 cells. [Ca²⁺]i was measured by fura2 fluorescence.

FIG. 35. Simultaneous knockdown of UCP2 and Nnt restores the wild-type response. Insulin secretion in response to glucose (0, 2, 10 mM) or tolbutamide (200 μM) from MIN6 cells transfected with nonsense siRNA (black bars, n=6), NntI siRNA (white bars, n=6), UCP2 siRNA (red bars, n=6), UCP2/Nnt siRNA (blue bars, n=6) and Gck siRNA (grey bars, n=6). The data are the mean±SEM of six wells and are representative of 3 separate experiments.

FIG. 36. Metformin restores insulin secretion in Nnt mouse pancreatic islets. Insulin secretion in response to glucose (1 or 20 mM) from islets isolated from wild-type or Nnt-G745D mice following 24 hr preincubation in the absence or presence of 15 μM metformin. The data are the mean±SEM of six wells (5 islets/well) and are representative of 3 separate experiments.

### DETAILED DESCRIPTION OF THE PREFERRED EMBODIMENTS OF THE INVENTION

The C57BL/6J mouse is recognized as a model animal for Type 2 diabetes. In the data described herein genetic mapping of F2 intercrossed C57BL/6J mice, physiological studies of whole mice, studies of isolated liver and pancreatic tissues, and candidate gene analyses of C57BL/6J mice are used to define more precisely the genetic basis of glucose intolerance in C57BL/6J mice. C57BL/6J mice showed normal insulin sensitivity and impaired insulin secretion. In β-cells, glucose failed to stimulate a rise in intracellular calcium and showed impaired ability to close K_{ATP} channels. Further investigations showed that there are three main genetic loci that influence the difference in glucose homeostasis. These loci were located on chromosomes 9, 11 and 13. Underlying the chromosome 13 locus was a strong candidate gene for impaired insulin secretion in type 2 diabetes, Nicotinamide Nucleotide Transhydrogenase (Nnt). Expression of Nnt is at least 7-fold and 5-fold lower in C57BL/6J liver and pancreatic islets. Sequencing of the coding region of Nnt in C57BL/6J mice identified two mutations in this gene that were related to the glucose intolerance. The first is a missense mutation (M35T) in the mitochondrial leader sequence of the Nnt precursor protein. The second is an in-frame 5-exon deletion that removes four predicted transmembrane helices and their connecting linkers.

The C57BL/6J mouse strain exhibits plasma glucose intolerance reminiscent of human Type-2 diabetes. The data discussed herein suggest a defect in β-cell glucose metabolism that results in reduced electrical activity and insulin secretion. In particular, the present invention is based on the discovery that Nnt is one of the genetic elements which can make a person susceptible to type 2 diabetes. An alteration to the Nnt gene makes an individual susceptible to developing type 2 diabetes. Nnt is a nuclear-encoded mitochondrial protein that is located in the inner mitochondrial membrane of eukaryotes and in the plasma membrane of some bacteria (for review, see Hoek et al., Biochem J 254:1-10 (1988)). It functions as a redox-driven proton pump, catalyzing the reversible reduction of NADP+ by NADH and conversion of NADH to NAD+. This is linked to proton translocation across the membrane according to the reaction:
NADH+NADP+H_{cystolic}⇄NAD+NADPH +Hₘₐₜᵣᵢₓ

Under physiological conditions, a marked proton gradient is present across the inner mitochondrial membrane so that the reaction is driven strongly to the right. This makes Nnt a very efficient generator of NADPH. Moreover, it couples the production of NADPH to the rate of mitochondrial metabolism, and hence to the production of reactive oxygen species (ROS) generated by the electron transport chain. Thus, Nnt is postulated to play an important role in the detoxification of ROS.

Nnt is a homodimer and the individual subunits (Mr ˜110 kDa) are composed of three domains (Hoek et al., Biochem J 254:1-10 (1988)). The N-terminal hydrophilic domain (I) binds NAD(H), the central hydrophobic domain (II) intercalates into the membrane (it consists of 14 transmembrane domains and forms the proton channel), and the C-terminal hydrophilic domain (III) binds NADP(H) (Yamaguchi et al., J Biol Chem 266:5728-5735 (1991)). Both domains I and III are located within the mitochondrial matrix. The 5-exon deletion found in the Nnt gene of C57BL/6 mice deletes part of domain I and the first four transmembrane domains and leads to a marked down-regulation of Nnt mRNA in liver and pancreatic islets.

In exploring the functional role of Nnt in detail, it was discovered that knockdown of Nnt in insulin-secreting MIN6 cells prevents elevation of intracellular calcium and insulin secretion in response to glucose, but not to the K_{ATP} channel blocker tolbutamide. Likewise, mice carrying mutations in Nnt have impaired glucose tolerance and loss of both glucose-dependent insulin secretion and ATP production in isolated pancreatic islets. These data are consistent with the idea that impaired Nnt function reduces β-cell metabolism, thereby preventing closure of K_{ATP} channels in response to glucose, calcium influx and insulin secretion.

When insulin secretion from isolated pancreatic islets was examined over a wider range of glucose concentrations it was seen that whereas glucose concentrations above 5 mM stimulate secretion in wild-type pancreatic islets, there is no significant release in response to either 5 or 10 mM glucose in islets isolated from Nnt mutant mice N68K and G745D. At 20 mM glucose, however, there was a small but significant increase in insulin secretion from both N68K and G745D islets. These results explain the insulin secretion observed in vivo, and also explain why the mice do not suffer from overt diabetes, but only from impaired glucose tolerance.

Additional studies described herein prove that Nnt is the gene underling inappropriate glucose homeostasis in C57BL/6J mice (see Freeman et al. Diabetes 55:2153-2156 (2006)). The examples below provide clear evidence of a change in mitochondrial membrane potential as a result of reducing levels of Nnt or mutating Nnt.

Consistent with a role for Nnt in production of reduced glutathione it was shown that reduced glutathione levels are lower in Nnt mutant pancreatic islets, supporting the hypothesis that ROS is part of the mechanism ultimately controlling insulin secretion and that Nnt mutants exert their effects through changes in ROS levels.

UCP2 is upregulated in Nnt pancreatic islets where Nnt has been knocked down. SiRNA based studies showed that the calcium response to glucose was unaffected by knockdown of UCP2 alone. Knockdown of Nnt alone almost completely abolished the calcium response, as previously reported. However, simultaneous knockdown of UCP2 and Nnt reversed the impaired secretory response to glucose observed for Nnt knockdown alone. These and other data shown herein support a role for Nnt acting by stimulating UCP2 function, either via enhanced expression of UCP2, or by increasing UCP2 activity directly, or both.

The identification of the functional role of Nnt in determining susceptibility to type 2 diabetes provides new diagnostic, prevention and treatment tools for type 2 diabetes and related diseases. Related diseases include those diseases and conditions which are treated or ameliorated by modulation of Nnt activity and/or expression. These diseases and conditions include, disorders relating to oxidative stress such as motor neurone disease and aging, as well as to glucose metabolism, insulin secretion, vesicle transport in secretory cells, pancreas and hepatocyte activity, dyslipidemia and obesity. Disorders in which insulin concentrations are severely reduced are insulin-dependent diabetes mellitus (Type 1 diabetes) and some other conditions such as hypopituitarism. Insulin levels are raised in non-insulin-dependent diabetes mellitus (Type 2 diabetes), obesity, insulinoma and some endocrine dysfunctions such as Cushing's syndrome and acromegaly.

The cDNA sequence (e.g., GenBank Accession No. NM_{—}012343 and NM_{—}182977, homo sapiens NNT mRNA) and the related amino acid sequences are provided herein as SEQ ID NO.1-2 (sequences for NM_{—}012343 ) and 3-4 (sequences for NM_{—}182977). Mouse homologs of these sequences are also known to those of skill in the art. For example, the mouse homolog is depicted in FIG. 7 (the figure is incorporated by reference) of Toye et al., Diabetologia, April; 48(4):675-86. Epub 2005 Feb. 24 (2005). The human and mouse sequences are highly homologous, and this degree of identity provides a rational for the prediction that the genetic evidence from mouse models presented herein predicts the same genetic phenomenon in humans.

For diagnostic purposes, a subject can be examined for the allele of their Nnt gene as a step in determining whether the subject is likely to be susceptible to developing type 2 diabetes. For example, the Nnt cDNA sequence of such a subject can be determined and the deduced amino acid sequence can be compared to SEQ ID NO:2 or SEQ ID NO:4. If a mutation or polymorphism is detected at the amino acid level, especially if the mutation is one other than a conservative substitution, the individual can be identified as susceptible to developing type 2 diabetes. A “patient” or “subject” to be treated or diagnosed by a disclosed method can mean either a human or non-human animal.

Susceptibility may also be determined by measuring the mRNA or protein level of Nnt. A lack of expression of the proper form of Nnt, at either the mRNA level or the protein level indicates susceptibility to developing type 2 diabetes. In the Examples herein Western blotting, PCR and protein gels are used to determine the level of expression of Nnt. Such techniques will be readily adaptable for the diagnostic aspects of the present invention. The expression level determined can be compared to the normal range of level of expression and an expression level that is changed from that of the normal range indicates susceptibility to developing type 2 diabetes. The normal range of level of expression can be readily established by measuring the expression level in a suitable number of type 2 diabetes-free individuals. Given that the cDNA and amino acid sequences of Nnt are known, one of ordinary skill can readily design probes and primers and generate antibodies to practice the method described above.

It may also be desirable to determine the nature of the Nnt gene expression in order to more particularly tailor therapies for a particular individual. Some therapeutic agents are only effective in patients having a selected variant of a certain gene. In this embodiment, a subject in need of treatment provides a DNA sample from which the DNA sequences of Nnt is determined. The outcome determines which therapeutic agent is administered to the patient.

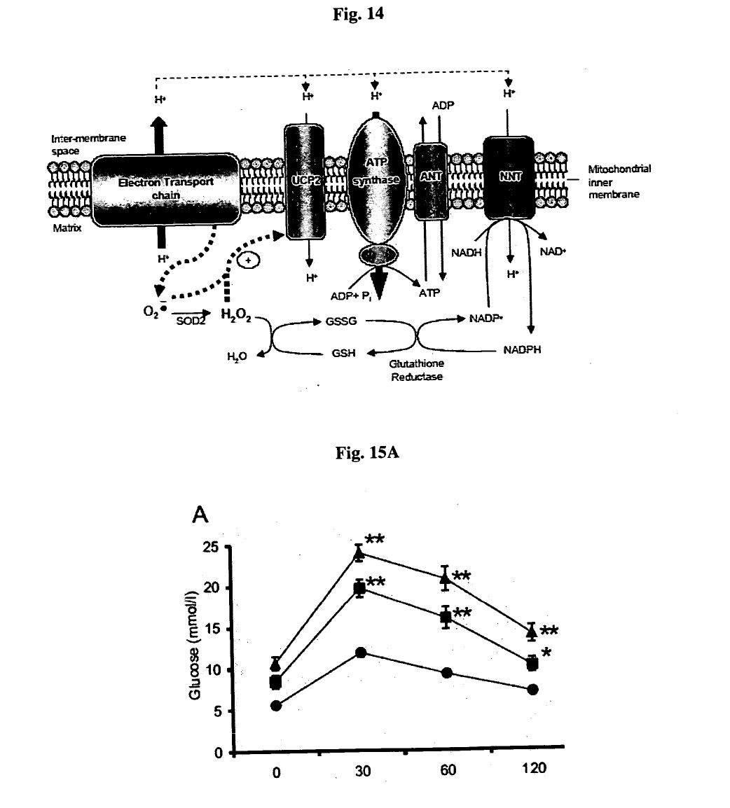

From the perspective of prevention or treatment of type 2 diabetes, agents that augment, potentiate or otherwise increase the activity of Nnt will be potential agents for the prevention or therapy of type 2 diabetes agents. As indicated in the discussion of example 2 Nnt is involved in the detoxification of reactive oxygen species generated by the electron transport chain. The process by which this occurs is shown in FIG. 14. It requires a continuous supply of NADPH, which is provided in the mitochondria by the activity of Nnt, which converts NADP and NAD to NADPH and NADH, respectively. In the absence of a proper Nnt, there is excess H₂O₂, which leads to deleterious effects in the pancreatic β-cells. If the activity of Nnt can be stimulated artificially, the effect of the disease might be ameliorated.

Another aspect of the invention is to identify those agents that can stimulate, augment or otherwise potentiate the activity of Nnt. Given that the cDNA and amino acid sequences of Nnt are known, one of ordinary skill can readily screen for agents that interact with Nnt. For example, one can use a cell culture system in which cells express Nnt (including also cell-free assays, such as assays employing isolated mitochondria). These cells (or isolated organelles, such as mitochondria) can be exposed to a test agent and the presence and absence of an agent/Nnt complex is determined. An in vitro system in which a Nnt protein can be exposed to a test agent directly can also be used to screen for agents that affect its activity. In such screening method, not only the human Nnt but also mouse, or genes from other mammalian species can be used. Fragments of these proteins that include the VPS10 domain or other domains can also be used. In addition, the cells used in the screening methods may be those that contain wild-type Nnt or alternatively may contain a mutant Nnt such as one of the mutants described herein as causing susceptibility to diabetes. Where a mutant Nnt is used, a screening assay that identifies an output that is improved as a result of the present of the test agent will be particularly useful. In certain embodiments, the screening assays employ a mutant Nnt gene that produces a polymorphism or other mutation in the wild-type NNT protein sequence of for example SEQ ID NO:2 or SEQ ID NO:4.

As such a preferred aspect of the present invention contemplates the use of NNT proteins and active fragments thereof in the screening of compounds that modulate (increase or decrease) the activity, expression or targeting of the Nnt protein into the mitochondria. These assays may make use of a variety of different formats and may depend on the kind of “activity” for which the screen is being conducted. Contemplated functional “read-outs” include NNT protein binding to a substrate; a reduced insulin secretion, a glucose-dependent increase in [Ca²⁺]i in cells, an increased glucose intolerance, impaired glucose dependent β-cell electrical activity, a decrease in β-cell ATP production, or enhanced K_{ATP} channel activity. Methods of monitoring such parameters are known to those of skill in the art and are described in the Examples below. For example, insulin secretion can be determined using enzyme-linked immunoassay techniques, intracellular calcium levels can be measured using imaging studies with a calcium indicator such as Fura 2, methods of monitoring glucose tolerance in a whole animal are described under the heading “intraperitoneal glucose tolerance tests” in the Examples. Whole-cell K_{ATP} currents can be measured using a perforated patch configuration of the patch-clamp technique [Sakura et al., Diabetologia 41:654-659 (1998)] (see Examples.) Similar techniques for monitoring the characteristics of type 2 diabetes will be well known to those of skill in the art.

All types of assays for identifying agents that modulate the activity of Nnt are contemplated by the inventors. Such assays include, but are not limited to, assays which measure Nnt biological activity, assays which measure expression of Nnt (preferably employing the promoter gene sequence of these proteins linked to a reporter gene) or “in silico” assays which use computational models of the protein to predict compounds which will modulate the protein biological activity or expression. The assays are designed to identify ligands and modulators which are potential therapeutic agents, or analogs thereof, which have utility in the treatment of type II diabetes and related diseases.

As used herein, the term “modulate the activity (or expression or targeting) of Nnt” means increase or decrease the activity (or expression or targeting) of Nnt. Increasing the activity (or expression or targeting) of Nnt will be useful in the treatment of type II diabetes, for example, where the increase of Nnt in the pancreatic β-cells will result in a beneficial increase in glucose-stimulated insulin secretion, and the like. Conversely, a decrease in the activity (or expression or targeting) of Nnt also will be useful particularly in those circumstances where the subject is suffering from e.g., hyperinsulinemia. mRNA or protein expression assays are also useful for identifying compounds which can modulate (i.e. up regulate or down regulate or increase or decrease) expression of the gene, including compounds which modulate the activity of transcriptional regulators of Nnt. Such expression assays typically include an expression construct comprising the promoter region (5′UTR and associated genomic sequence) of the gene linked to a reporter gene. Potential therapeutic agents, or analogs thereof, are identified by their ability to modulate expression of the gene in question. Those skilled in the art are capable of identifying transcription factors which are responsible for regulating transcription of the gene in question.

a. Assay Formats.

The present invention provides methods of screening for modulators of Nnt protein activity, expression or targeting of Nnt into the mitochondria by effects of Nnt in a cell in the presence and absence of the candidate substance and comparing such results. It is contemplated that such screening techniques will prove useful in the general identification of a compound that will serve the purpose of altering the insulin secretion in a cell and such compounds will ultimately be useful as therapeutic agents. For example, those agents that increase the activity, expression or transport of Nnt will be useful in the treatment of diabetes because it is shown herein that mutations or polymorphisms in Nnt result in type II diabetes. However, it is contemplated that agents that decrease the activity expression or transport of Nnt also will be useful in the treatment of e.g., hyperinsulinemia. Thus in certain embodiments, it will be desirable to identify stimulators of Nnt activity, expression or targeting of the Nnt into mitochondria and in other embodiments it will be desirable to identify inhibitors of Nnt activity, expression or targeting.

In the screening embodiments, the present invention is directed to a method for determining the ability of a candidate substance to alter the Nnt protein expression or activity or mitochondrial targeting of Nnt in cells that either naturally express Nnt protein or have been engineered to express Nnt protein. Methods of engineering cells to transfect them with a polynucleotide that encodes a given protein are routine in the art.

An alteration in Nnt protein activity, expression or mitochondrial targeting in the presence of the candidate substance will indicate that the candidate substance is a modulator of the activity. The effects of the candidate substance on insulin secretion, changes in [Ca²⁺]i, changes in glucose dependent β-cell electrical activity, changes in β-cell ATP production, or changes in K_{ATP} channel activity will be indicative of the candidate substance being a modulator of Nnt activity, expression or targeting .

While the above method generally describes a Nnt protein activity, it should be understood that a candidate substance may be an agent that alters the production of Nnt protein, thereby increasing or decreasing the amount of Nnt protein present as opposed to the per unit activity of the Nnt protein.

To identify a candidate substance as being capable of modulating Nnt protein activity, one would measure or determine Nnt protein activity in the absence of the added candidate substance. One would then add the candidate inhibitory/stimulator substance to the cell and determine the Nnt protein activity, expression or mitochondrial targeting or other characteristic output being monitored in the presence of the candidate inhibitory substance. A candidate substance which is inhibitory would decrease the Nnt protein activity, expression or mitochondrial targeting, relative to the same parameter in its absence, whereas a stimulator will increase such an activity, expression or mitochondrial targeting, relative to the parameter in its absence.

b. Candidate Substances.

As used herein the term “candidate substance” refers to any molecule that is capable of modulating Nnt protein activity, expression or mitochondrial targeting. The candidate substance may be a protein or fragment thereof, a small molecule inhibitor, or even a nucleic acid molecule. It may prove to be the case that the most useful pharmacological compounds for identification through application of the screening assay will be compounds that are structurally related to other known agents typically used type II diabetes. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential.

The present invention provides screening assays to identify agents which inhibit or otherwise treat the indicia of type II diabetes. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known agents that are used in the intervention of type II diabetes.

The candidate screening assays are simple to set up and perform. Thus, in assaying for a candidate substance, after obtaining a cell expressing functional Nnt protein, one will admix a candidate substance with the cell, under conditions which would allow measurable Nnt protein activity or expression or mitochondrial targeting to occur. In this fashion, one can measure the ability of the candidate substance to stimulate the biological effect mediated by Nnt activity of the cell in the absence of the candidate substance. While the expression, activity or mitochondrial targeting of the Nnt itself can be monitored, it should be appreciated that biological properties such as determination of insulin secretion using assays that employ ELISA techniques commercially available in kits from Mercodia, Winston Salem, N.C. (e.g., commercial specific enzyme immunoassay for quantification of human C-peptide; insulin or proinsulin ELISAs) are readily available. In the candidate screening assays H₂O₂/free radical measurement may be measured using kits (kit available from Molecular Probes-Invitrogen) or reporter molecule undergoing conformational change in the presence of free radical/H₂O₂ (quantitative fluorescent output). Such an assay could be performed on isolated organelles (mitochondria), cell lines, or on whole tissues. Changes in membrane potential or in [Ca2+] in cells expressing K_{ATP} channels (e.g., β-cells); O₂ uptake (coupled with fluorescence); glucose utilization (coupled with fluorescence); blood glucose determinations (whole animal); UCP2 activity; ATP production; NADP/NAD status of a cell; in vitro protein assays; and screens for novel ion channel genes (Flipper technology) can also be used as outputs for the screening assays. will be useful outputs for the screening assays used herein. Likewise, in assays for inhibitors of Nnt, after obtaining a cell expressing functional Nnt protein, the candidate substance is admixed with the cell. In this fashion the ability of the candidate inhibitory substance to reduce, abolish, or otherwise diminish a biological effect mediated by Nnt protein from the cell may be detected.

“Effective amounts” in certain circumstances are those amounts effective to reproducibly alter a given Nnt-mediated event e.g., insulin secretion, from the cell in comparison to the normal levels of such an event. Compounds that achieve significant appropriate changes in such activity will be used.

Significant changes in Nnt protein activity or function or mitochondrial targeting of at least about 30%-40%, and most preferably, by changes of at least about 50%, with higher values of course being possible.

Proteins are often used in high throughput screening (HTS) assays known in the art, including melanophore assays to investigate receptor ligand interactions, yeast based assay systems and mammalian cell expression systems. For a review see Jayawickreme and Kost, Curr. Opin. Biotechnol. 8:629-634 (1997). Automated and miniaturized HTS assays are also contemplated as described for example in Houston et al., Curr. Opin. Biotechnol. 8:734-740 (1997). Such high throughput screening techniques may be used for screening for agents that modulate Nnt activity.

There are a number of different libraries used for the identification of small molecule modulators including chemical libraries, natural product libraries and combinatorial libraries comprised of random or designed peptides, oligonucleotides or organic molecules. Chemical libraries consist of structural analogs of known compounds or compounds that are identified as hits or leads via natural product screening or from screening against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63-68 (1998). Test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof. Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8:701-707 (1997). Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection; Bethesda, Md., NCI's Developmental Therapeutics Program, or the like. A candidate modulator identified by the use of various libraries described may then be optimized to modulate activity of Nnt protein through, for example, rational drug design.

c. In Vitro Assays.

In one particular embodiment, the invention encompasses various binding assays. These can include screening for modulators of Nnt activity using in vitro cellular assays or assays may be performed on isolated mitochondria. In some embodiments, agents that bind to Nnt may be useful. In such binding assays, the Nnt protein or a fragment thereof may be either free in solution, fixed to a support, expressed in or on the surface of a cell. Either the polypeptide or the binding agent (i.e., the modulator to which the Nnt binds) may be labeled, thereby permitting determination of binding.

Such assays are highly amenable to automation and high throughput. High throughput screening of compounds is described in WO 84/03564. Large numbers of test compounds are synthesized on a solid substrate, such as plastic pins, 96-well plate, beads or some other solid surface. The test compounds are reacted with Nnt protein and washed. Bound polypeptide is detected by various methods. Combinatorial methods for generating suitable test compounds are specifically contemplated.

Purified Nnt protein or a binding agent can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the Nnt protein active region to a solid phase.

Other forms of in vitro assays include those in which functional readouts are taken. For example cells in which a wild-type or mutant Nnt protein polypeptide is expressed can be treated with a candidate substance. In such assays, the substance would be formulated appropriately, given its biochemical nature, and contacted with the cell. Depending on the assay, culture may be required. The cell may then be examined by virtue of a number of different physiologic assays, as discussed above. Alternatively, molecular analysis may be performed in which the cells characteristics are examined. This may involve assays such as those for protein expression, enzyme function, substrate utilization, mRNA expression (including differential display of whole cell or polyA RNA) and others.

In exemplary embodiments, a high throughput assay to screen for compounds that affect NNT activity may be based on the method described by Farrelly et al. Analytical Biochemistry 293:269-276 (2001). Exemplary such HTS assays are described in Example 9 herein below. A yeast assay for high throughput screening can be based on the method described by Farrelly et al. Analytical Biochemistry 293:269-276 (2001) and explained in further detail in Example 10.

d. In Vivo Assays.

The present invention also encompasses the use of various animal models. Animal models for Type II diabetes are well known to those of skill in the art and exemplary such models are described in the Examples herein below. These models afford the skilled artisan an excellent opportunity to examine the function of Nnt protein and its modulators in a whole animal system where it is normally expressed. Thus, agents can be identified in initial in vitro screens as discussed above and then further tested in whole animal screens. In this manner, one can achieve test results with the candidate compounds that will be highly predictive of efficact in humans and other mammals, and helpful in identifying potential therapies.

Treatment of animals with test compounds will involve the administration of the compound, in an appropriate form, to the animal. Administration will be by any route that can be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood, cerebrospinal fluid (CSF) or lymph supply.

Determining the effectiveness of a compound in vivo may involve a variety of different criteria. Such criteria include, but are not limited to, survival, increased activity level, improvement in immune effector function and improved food intake; a change in glucose tolerance, thermoregulation and the like as described e.g., in Example 2.

Agents identified from such screening assays can then be formulated into pharmaceutical compositions for use as therapeutic (or prophylactic) agents. Such agents may be orally available small molecule compounds. In an alternative embodiment, such compositions are selected from among small molecules, antisense molecules, siRNA, therapeutic antibodies and the like directed against the mutant NNT whose expression in mitochondria of β-cells is indicative of susceptibility to type 2 diabetes. In some embodiments a gene therapy vehicle (plasmid, viral or non-viral (lipid based) vector) may be used to deliver a copy of the Nnt gene to a cell for therapeutic expression of the respective proteins. Therapeutic compounds may be delivered orally, intravenously, by inhalation, and/or by any other of the means well known to those in the art.

Any of the therapeutic agents identified by the above screens may be administered alone as therapeutic agents or alternatively may be administered in combination with other known treatments for type 2 diabetes, including one or more of diet, exercise, administration of a sulfonylurea such as tolbutamide, chlorpropamide, glibenclamide, glipizide, glimeperide, and gliclazide or a biguanide such as metformin, phenformin and buformin. Metformin is widely used to treat type-2 diabetes. In the present invention it was demonstrated that preincubation in 15 μM metformin surprisingly restored glucose-stimulated insulin secretion in pancreatic islets from the Nnt-G745D mutant mouse, while having little effect on wild-type (WT) pancreatic islets. Combination therapy with metformin and a therapeutic agent identified in the present invention is contemplated to be particularly useful.

In another therapeutic embodiment, it is contemplated that a wild-type Nnt activity may be delivered to a mammal suffering from type 2 diabetes where the wild-type enzyme is delivered in the form of a therapeutic cell that expresses the wild-type enzyme. The cell may be used as a “patch” and implanted into the animal. As it is shown herein in the examples that although Nnt is widely expressed in human tissues (Arkblad et al., 2002), it appears that only the β-cell is particularly sensitive to mutations and deletion of the Nnt gene, it would be desirable to prepare β-cells that express wild-type Nnt and transplant such cells into subjects suffering from type 2 diabetes as a result of a mutation in Nnt.

In order to carry out the methods of the present invention, the skilled artisan may employ a wide variety of tools for use in research which employ Nnt. For example, purified Nnt genes or proteins, recombinant cells containing additional copies of such gene(s), antibodies to the Nnt proteins (humanized, therapeutic or otherwise) and transgenic animals, such as mice created to have non-functional forms of the gene (knock-out or knock-down) or recombinant mice having additional copies of the gene(s) may be useful. Recombinant techniques that will allow for the purification of Nnt genes, preparation of recombinant cells that express Nnt proteins, preparations of isolated recombinant such proteins are routine well known to those of skill in the art. See for example, Molecular Cloning: A Laboratory Manual, Sambrook et al., Cold Spring Harbor Press, (2001) a 3-volume treatise on recombinant techniques. These documents also provide detailed “cookbook” teachings of methods for the preparation of recombinant cells that express a desired protein. Any recombinant cell can be used. The cell may be a pancreatic β-cell, or any other mammalian cells that can be transfected with an Nnt-encoding nucleic acid. Mammalian cells useful in recombinant protein production include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Genetic and Physiological Study of Impaired Glucose Homeostasis Control in C57BL/6J Mice

In the present example, it is shown that there are 3 genetic loci that may be responsible for the impaired glucose tolerance in C57BL/6J mice which are a recognized model for human Type-2 diabetes. The nuclear-encoded mitochondrial proton pump, Nnt, lies within one of these loci. Expression of Nnt is >7-fold and 5-fold lower in C57BL/6J liver and pancreatic islets. There is a missense mutation in exon 1 and a multi-exon deletion in the C57BL/6J gene. Glucokinase lies within the Gluchos2 locus and shows reduced enzyme activity in liver. The C57BL/6J mouse strain exhibits plasma glucose intolerance reminiscent of human Type-2 diabetes. The data in this Example show that a defect in β-cell glucose metabolism that results in reduced electrical activity and insulin secretion. Nnt is a novel candidate gene for contribution to glucose intolerance through reduced β-cell activity.

A. Research Design and Methods

i. Animals

Mice were kept in accordance with UK Home Office welfare guidelines and project license restrictions. C57BL/6J mice were obtained from Jackson Laboratory (Maine, USA) in 1999. C3H/HeH mice were from the Harwell (Oxfordshire, UK) stock. Mice were maintained under controlled conditions and were fed ad libitum on a commercial diet (maintenance chow, Special Diet Services, Essex, UK) containing 2.6% saturated fat.

ii. Genotyping

Genomic DNA was extracted from mouse tail tissue using a Qiagen DNeasy tissue kit (GmbH, D-40724 Hilden, Germany). Mice were genotyped by SSLP analysis. Primer sequences were obtained from published records [Dietrich et al., Nat Genet 7 (Special issue): 220-245 (1994)]. PCR products were analysed using an ABI 377 sequencer and Genescan and Genotyper software and protocols (Applied Biosystems, EUROPE). Markers were spaced at a median interval of 11 cM, an average interval of 14.6 cM and the largest interval was 46.7 cM

iii. Linkage Analysis

Phenotype and genotype data were maintained in Microsoft Excel, SPSS and MapManager QTX ([Manly et al., Mamm Genome 12:930-932 (2001)]; see http://mapmgr.roswellpark.org/mimQTX.html) formats. Genetic maps were constructed using the published map order of markers (http://www.informatics.jax.org). Linkage between markers and phenotypes was evaluated using a single marker, and interval mapping features of Map Manager. Thresholds for defining linkage were as outlined by Lander and Kruglyak [Lander et al., Nat Genet 11:241-247 (1995)], with additional permutation tests [Manly et al., Mamm Genome 12:930-932 (2001)] where stated. Empirical thresholds were based on analysis of 1000 permutations of the original data set. Free model permutation derived thresholds were LRS 15.7 (LOD 3.4) for T0 glucose, LRS 16.1 (LOD 3.5) for T30 glucose, LRS 15.5 (LOD 3.4) for T60 glucose, LRS 15.7 (LOD 3.4) for AUC glucose and LRS 16.9 (LOD 3.7) for T30 insulin.

iv. Multilocus Model

Multiple regression was performed by univariate ANOVA using the GLM feature of SPSS. F statistics were based on type III sums of squares. All markers that exceeded LOD 1.9 suggestive linkage threshold [Lander et al., Nat Genet 11:241-247 (1995)] were included in the regression model. Terms not significant at the 0.1% level were sequentially eliminated to produce the final multiple regression model. C57BL/6J homozygotes were coded 0.5, C3H/HeH homozygotes were coded −0.5 and heterozygotes were coded 0 in all regression analyses.

v. Intraperitoneal Glucose Tolerance Tests

Mice were fasted overnight, weighed, and a blood sample collected by tail venipuncture under local anaesthetic (Lignocaine, Biorex, Middlesex, UK) using Lithium-Heparin microvette tubes (Sarstedt, D-51588 Numbrecht, Germany) to establish a baseline glucose level “T0”. They were then injected intraperitoneally with 2 g glucose/kg body weight and blood samples taken at 15, 30, 60 and 120 min following injection to monitor the rate of glucose clearance. Plasma glucose and insulin was measured using a Beckman Glucose analyser and a Mercodia Ultra-sensitive Mouse ELISA kit.

vi. Insulin Tolerance Test

Insulin tolerance tests on free-fed mice were performed as described [Kooptiwut et al., Endocrinology 143:2085-2092 (2002)].

vii. Islet Cell Isolation

Mice were killed by cervical dislocation. The pancreas was removed and islets or dissociated islet cells were prepared and cultured as previously described [Rorsman et al., Pluugers Arch 405:305-309 (1985)]. Cells were exposed to glucose-free solution for 15 min prior to experiment.

viii. Insulin Secretion studies

Insulin secretion from isolated pancreatic islets (10 islets/well) was measured during 1 hr static incubations in Krebs-Ringer Buffer (in mmol/l: 118.5 NaCl, 2.54 CaCl₂, 1.19 KH₂PO₄, 4.74 KCl, 25 NaHCO₃, 1.19 MgSO₄, 10 HEPES, pH 7.4) containing either 2 mmol/l or 10 mmol/l glucose. Each glucose concentration was replicated 5-8 times. Samples of the supernatant were assayed for insulin. Insulin content was extracted using 95:5 ethanol/acetic acid. Insulin was measured using a Mouse Insulin ELISA kit (Mercodia, Sweden).

ix Fura-2 Imaging

Islet cells were cultured on 35 mm Fluorodishes (World Precision Instruments, Stevenage, UK) and incubated with 3 μM Fura-2-AM (Molecular Probes, Paisley, UK) for 40 min at 37° C. They were imaged at room temperature (20-24° C.) using an IonOptix fluorescence system (Boston, Mass.), with 340 nm and 380 nm dual excitation. The 510 nm emission ratio was collected at 1 Hz. Background subtraction was performed by measuring fluorescence from a cell-free region in the field of view. Cells were perfused continuously with extracellular solution (as in perforated-patch experiments), plus glucose or tolbutamide as indicated. Only data from cells that responded to 500 μmol/l tolbutamide were analysed.

x. Electrophysiology

Glucose concentration-response relations for whole-cell K_{ATP} currents were measured using the perforated patch configuration of the patch-clamp technique [Sakura et al., Diabetologia 41:654-659 (1998)]. The pipette solution contained (mmol/l): 70 K2SO4, 1 CaCl₂, 1 MgCl2, 10 NaCl, 10 HEPES (pH 7.2 with KOH), and amphotericin B (0.24 mg/ml). The extracellular (bath) solution contained (mmol/l): 137 NaCl, 5.6 KCl, 2.6 CaCl₂, 1.2 MgCl2, 10 HEPES (pH 7.4 with NaOH) plus glucose as indicated. Currents were filtered at 3 kHz and digitised at 1 kHz. The K_{ATP} component of the whole-cell current was taken as the current blocked by 200 μmol/l tolbutamide.

The ATP sensitivity of K_{ATP} channels was measured at −60 mV in inside-out patches. Currents were digitized at 5 kHz and filtered at 2 kHz. The pipette (extracellular) contained (mmol/l): 140 KCl, 2.6 CaCl₂, 1.2 MgCl2, 10 HEPES (pH 7.4 with KOH). The bath (intracellular) solution contained (mmol/l): 107 KCl, 1 MgCl2, 1 CaCl₂, 10 HEPES (pH 7.2 with KOH), and K2ATP as indicated.

Concentration-response relations were obtained by alternating test solutions with control (glucose- or ATP-free) solution. The control conductance was taken as the mean of that obtained in control solution before and after application of the test agent. The current in the presence of glucose or nucleotide (I) is plotted as a fraction of that obtained in control solution (IC). Data were fit with the Hill equation:
I/IC=1/(1+([X]/IC50)h)
where [X] is the concentration of glucose or ATP, IC50 is the concentration giving half-maximal inhibition of the current and h is the Hill coefficient. Data are given as mean±one S.E.M.

xi Quantitative RT-PCR

Quantitative RT-PCR was performed using ABI SYBR Green on an ABI PRISM 7700 Sequence Detection System (Applied Biosystems). To quantitate Nnt (nicotinamide nucleotide transhydrogenase) it was PCR amplified from cDNA (RT-PCR) together with an endogenous control Gapdh (glyceraldehyde-3-phosphate dehydrogenase). RT-PCR primers listed in 5′ to 3′ orientation are: for Gapd

Gapd888F—CCTGCGACTTCAACAGCAACT (SEQ ID NO:5) and

Gapd979RV—CCAGGAAATGAGCTTGACAAA (SEQ ID NO:6)

for Nnt:

NntXn5intF—GATAGTTGGTGGTGGCGTTG (SEQ ID NO:7) and

NntXn6intR—GATGTCCACCTCCTTGCACT (SEQ ID NO:8).

The t-tests for significant differences between strains are based on RQ (Nnt/Gapd) values. RRQ (or Relative RQ) is the ratio of the C3H/HeH RQ relative to that for C57BL/6J.

B. Results

i. Whole-Animal Studies.

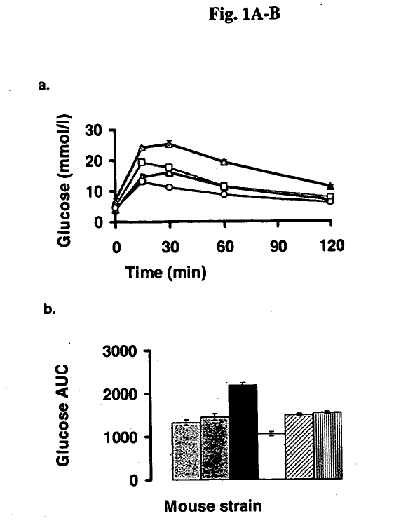

To identify suitable strains for mapping loci that determine obesity-independent impaired glucose tolerance, 4 non-obese inbred mouse strains (C57BL/6J, C3H/HeH, DBA/2 and BALB/c) were surveyed for variation in glucose tolerance during an Intraperitoneal glucose tolerance test (IPGTT). Male mice were less glucose tolerant than female (data not shown). Male C57BL/6J mice were also significantly less glucose tolerant than other strains (FIG. 1A; p<0.05), consistent with previous studies [Kaku et al., Diabetes 37:707-713 (1988); Kayo et al., Comp Med 50:296-302 (2000)]. Therefore, we used genetic analysis to identify loci that determine glucose homeostasis in 260 F2 male mice produced by intercrossing first the C57BL/6J strain with a control strain (C3H/HeH), and then their F1 progeny.

To elucidate whether epigenetic effects determine C57BL/6J glucose intolerance, the F1 offspring of C57BL/6J mothers were first compared with those of C3H/HeH mothers (FIG. 1B). No significant differences were observed between male (n=125) and female mice (n=91, p<0.05). F1 males (n=125) had significantly (p<0.0001) lower plasma glucose than their C57BL/6J (B, n=10), but not their C3H/HeH, parents (C, n=10) (FIG. 1B). Then the F2 phenotypes were examined in the four possible F1 cross combinations (CB×CB, CB×BC, BC×CB and BC×BC) in order to evaluate epigenetic effects in this generation. The T30, T60, T120 min and AUC glucose phenotypes were weakly significantly (close to the <0.05 threshold) different in some, but not all, of the pair-wise comparisons. For T0 two of the 6 phenotype comparisons were significantly different at the p=<0.01 level and one at p=<0.05 (data not shown). Thus there may be some weak epigenetic effects that influence in particular the T0 phenotype. All four cross combinations are equally represented in the F2 population for linkage analysis.

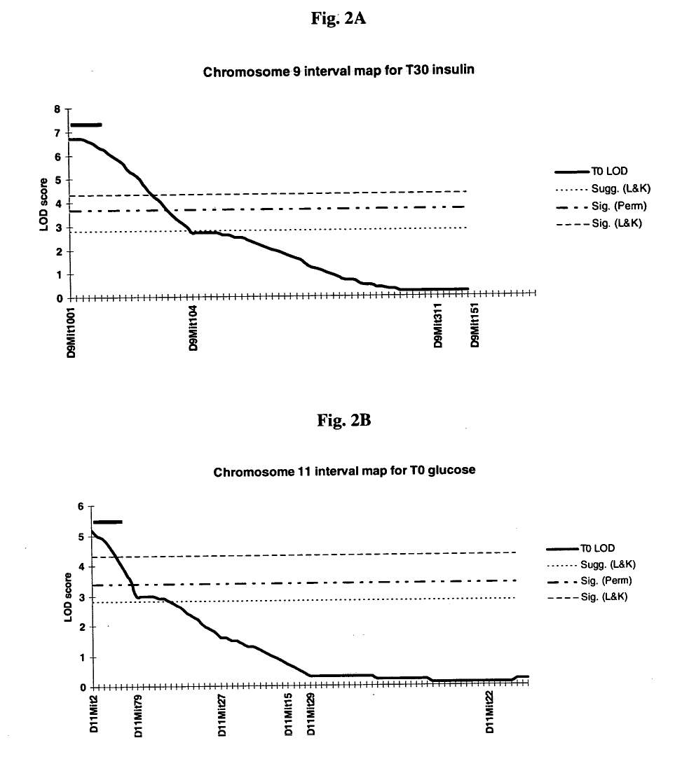

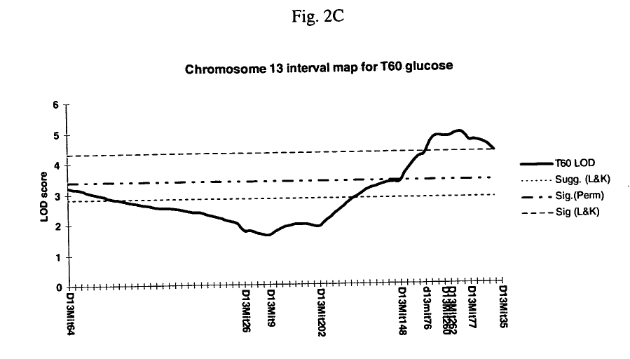

To identify contributing loci, 260 F2 male mice were genotyped with 84 markers [Dietrich et al., Nat Genet 7 (Special issue): 220-245 (1994)], providing genome coverage at an average intermarker distance of 15 cM. Plasma glucose levels during an IPGTT were significantly higher at T30 and T60 in the C57BL/6J strain, and were linked to D13Mit77 (LRS 22.8) (Table 1 and FIG. 2). This locus was named “Gluchos1” for glucose homeostasis locus-1.

TABLE 1
Free model LOD score profile across autosomes for T0,
T30, T60, AUC glucose and T30 insulin measurements.

| Plasma | Chromosome | | | | | | |
|---|---|---|---|---|---|---|---|
| trait^{a} | (Locus) | LRS (LOD)^{b} | % | p^{c} | CI^{d} | Add^{e} | Dom^{f} |
| T0 glucose | 11 (D11Mit2) | 23.7 (5.2) | 9 | 1E−05 | 23 | −0.78 | −0.32 |
| (mmol/l) | | | | | | | |
| T30 | 13 (D13Mit77) | 22.8 (5) | 8 | 1E−05 | 24 | −2.16 | −0.11 |
| glucose | | | | | | | |
| (mmol/l) | | | | | | | |
| T60 | 13 (D13Mit262) | 22.1 (4.8) | 8 | 2E−05 | 25 | −1.9 | −0.5 |
| glucose | | | | | | | |
| (mmol/l) | | | | | | | |
| AUC | 13 (D13Mit77) | 21.3 (4.6) | 8 | 2E−05 | 26 | −180 | −19 |
| (min | | | | | | | |
| mmol/l) | | | | | | | |
| T30 | 9 (D9Mit1001) | 30.9 (6.7) | 24 | 2E−07 | 20 | 0.19 | −0.15 |
| insulin | | | | | | | |
| (mg/dl) | | | | | | | |

| Plasma | Chromosome | Phenotype at closest marker^{g} | | | | | |
|---|---|---|---|---|---|---|---|
| trait^{a} | (Locus) | C3H (n) | | F1 (n) | | C57BL/6J (n) | |
| T0 | 11 (D11Mit2) | 4.91 ± 1.87 | (66) | 5.4 ± 1.89 | (139) | 6.44 ± 1.81 | (68) |
| glucose | | | | | | | |
| (mmol/l) | | | | | | | |
| T30 | 13 (D13Mit77) | 17.05 ± 5.14 | (71) | 18.96 ± 5.16 | (126) | 21.39 ± 5.2 | (64) |
| glucose | | | | | | | |
| (mmol/l) | | | | | | | |
| T60 | 13 (D13Mit262) | 13.6 ± 4.75 | (72) | 14.97 ± 4.71 | (132) | 17.36 ± 4.71 | (66) |
| glucose | | | | | | | |
| (mmol/l) | | | | | | | |
| AUC | 13 (D13Mit77) | 1535.76 ± 444 | (70) | 1685.45 ± 444 | (125) | 1898.31 ± 444 | (64) |
| (min | | | | | | | |
| mmol/l) | | | | | | | |
| T30 | 9 (D9Mit1001) | 0.59 ± 0.24 | (23) | 0.3 ± 0.23 | (61) | 0.26 ± 0.27 | (29) |
| insulin | | | | | | | |
| (mg/dl) | | | | | | | |

N = 280 random mice for all glucose traits. N = 120 mice, 60 each from opposite extremes of AUC distribution for T30 insulin. Only significant (Lander and Kruglyak [23]) linkages are shown.
^{a}Measure of blood plasma trait at time indicated or Area Under Curve (AUC),
^{b}Likelihood ratio statistic (LOD score), % percentage of variance explained by this locus (at DnMitn micro-satellite marker name),
^{c}P, probability value,
^{d}Confidence Interval- the interval around the marker in which the QTL will localise in 95% of attempts to map it,
^{e}Additive regression coefficient,
^{f}Dominance regression coefficient,
^{g}Average ± S.D. phenotype values for the indicated locus marker in the three possible genotype states; C3H homozygous; F1 heterozygous and C57BL/6J homozygous (n = number of animals).

The fasting plasma glucose level was linked to chromosome 11 (D11Mit 2, LRS 23.7) and is designated Gluchos2 (Table 1 and FIG. 2). Plasma glucose at T15 and T120 were not significantly linked to any locus.

The plasma insulin level at T30 provides a measure of second-phase insulin secretion [Kaku et al., Diabetes 37:707-713 (1988); Kayo et al., Comp Med 50:296-302 (2000)]. It was significantly (p<0.05, R2=0.0433, R=0.208), but poorly, correlated to plasma glucose at the same IPGTT time point, and linked to a locus on chromosome 9 (D9Mit1001, LRS 30.9) which was named Gluchos3 (Table 1 and FIG. 2). Insulin was correlated with body weight (R2=0.2319, R=0.48) but body weight was not correlated with T30 glucose (R2=0.0196, R=0.014).

Glucose intolerance can arise through the co-ordinated action of several loci [Stoehr et al., Diabetes 49:1946-1954 (2000); Gauguier et al., Nat Genet 12:38-43 (1996)]. Therefore the role of gene interactions was explored. The combined action of Gluchos1 and 7 additional loci (suggestive of linkage to plasma glucose) explains 35% of the variation in AUC, which is a measure of postprandial plasma glucose clearance (Table 2).

TABLE 2
ANOVA Table for regression of multiple QTL on
plasma glucose tolerance AUC (minmmol/1).

| | | | | | | Exp |
|---|---|---|---|---|---|---|
| Factor | Type III SS | df | MS | F | P | (%) |
| Corrected | 22412056.41 | 24 | 933835.68 | 6.642044 | 7.56141E−16 | 41.36 |
| Model | | | | | | |
| Intercept | 427405402.26 | 1 | 427405402.26 | 3039.984 | 4.6828E−133 | |
| D13Mit77 | 3394911.79 | 2 | 1697455.90 | 12.07341 | 1.04285E−05 | 9.65 |
| D13Mit64 | 492705.72 | 2 | 246352.86 | 1.752221 | 0.175735623 | 1.53 |
| D19Mit41 | 1032581.65 | 2 | 516290.82 | 3.672195 | 0.026949558 | 3.15 |
| D13Mit64 * | 4354787.68 | 4 | 1088696.92 | 7.743517 | 7.2731E−06 | 12.05 |
| D19Mit41 | | | | | | |
| D2Mit200 | 101147.70 | 2 | 50573.85 | 0.359714 | 0.698274735 | 0.32 |
| D9Mit311 | 578235.94 | 2 | 289117.97 | 2.056394 | 0.130301167 | 1.79 |
| D2Mit200 * | 2895102.84 | 4 | 723775.71 | 5.147961 | 0.000548529 | 8.35 |
| D9Mit311 | | | | | | |
| D7Mit91 | 2844846.90 | 2 | 1422423.45 | 10.1172 | 6.19226E−05 | 8.22 |
| D6Mit268 | 3263969.15 | 2 | 1631984.57 | 11.60773 | 1.58957E−05 | 9.32 |
| D16Mit146 | 1839186.94 | 2 | 919593.47 | 6.540744 | 0.001732053 | 5.47 |
| Error | 31774385.23 | 226 | 140594.62 | | | |
| Total | 781242405.41 | 251 | | | | |
| Corrected | 54186441.64 | 250 | | | | |
| Total | | | | | | |

Factor is source of plasma glucose AUC variation, SS is type III sum of squares adjusting for all other terms in the model, df is degrees of freedom, MS is mean square, F is F statistic based on adjusted sum of squares, P is p-value based on the F distribution, Exp (%) is estimate of variance explained by a factor or ‘Eta square’, expressed in percentage terms [Exp % = 100 × SSfactor/(SSfactor + SSerror)].
R Squared = 0.414
(Adjusted R Squared = 0.351)

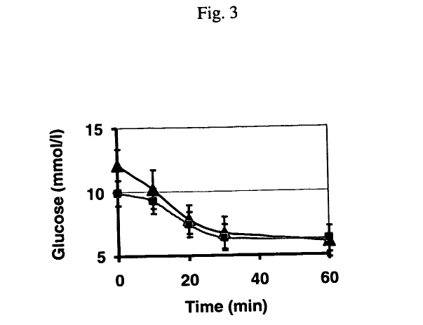

Insulin sensitivity was assessed in C57BL/6J mice using an intraperitoneal insulin tolerance test. C57BL/6J mice (n=10) had significantly higher free-fed plasma glucose prior to insulin injection than C3H/HeH mice (p<0.001; FIG. 3) and responded significantly better to insulin within the first 10 min (p<0.03). No significant difference (p>0.05) was seen between strains in plasma glucose levels in response to insulin at 10, 20, 30 and 60 min after insulin injection.

Therefore differences in insulin secretion were then assessed by carrying out an IPGTT, taking samples for insulin analysis at 0, 10, 20 and 30 min after glucose injection (Table 3). Insulin secretion by C57BL/6J mice is significantly less (for the same glucose challenge per g bodyweight) than for C3H/HeH mice. On average C57BL/6J mice were about 6 g lighter than C3H/HeH mice. Thus the present data favour a defect in insulin secretion rather than insulin action.

TABLE 3
Male C57BL/6J mice secrete less insulin than C3H/HeH
mice in response to a glucose challenge in an IPGTT.

| | Body weight | ng/ml plasma insulin at timepoints in an IPGTT ± SD | | | |
|---|---|---|---|---|---|
| Strain | g ± SD*** | 0 min** | 10 min** | 20 min** | 30 min* |
| C3H/HeH | 31.77 ± 0.99 | 0.46 ± 0.22 | 0.81 ± 0.50 | 1.00 ± 0.57 | 0.63 ± 0.44 |
| (n = 8) | | | | | |
| C57BL/6J | 25.45 ± 1.18 | 0.19 ± 0.00 | 0.19 ± 0.00 | 0.32 ± 0.32 | 0.23 ± 0.06 |
| (n = 8) | | | | | |

Mice were approximately 11.5 to 13 weeks old. The lower sensitivity of the insulin ELISA assays is 0.19 ng/ml and thus the levels of insulin in C57BL/6J mice at 0 and 10 minutes may actually be lower than as shown. Differences are significance at the values of
*≦0.05,
**≦0.01,
***≦0.001

ii Isolated Islet studies

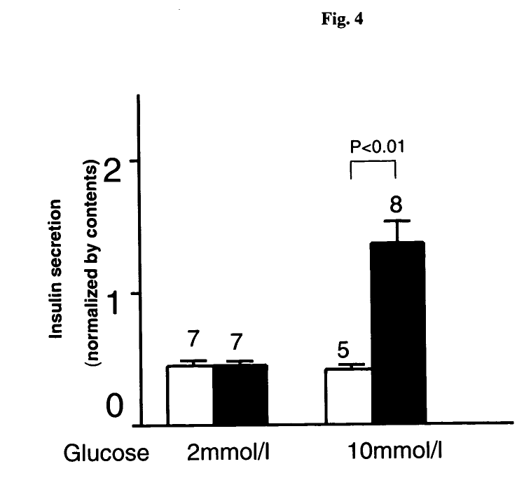

To test the idea that a defect in β-cell function underlies the glucose intolerance of C57BL/6J mice, the ability of glucose to stimulate insulin secretion from pancreatic islets isolated from C3H/HeH was compared to that of C57BL/6J mice. FIG. 4 shows there was no difference in basal secretion (at 2 mmol/l glucose) between the two mouse strains, but that insulin secretion in response to 10 mmol/l glucose is impaired in C57BL/6J islets in comparison with C3H/HeH islets (p<0.01). The data is representative of three experiments.

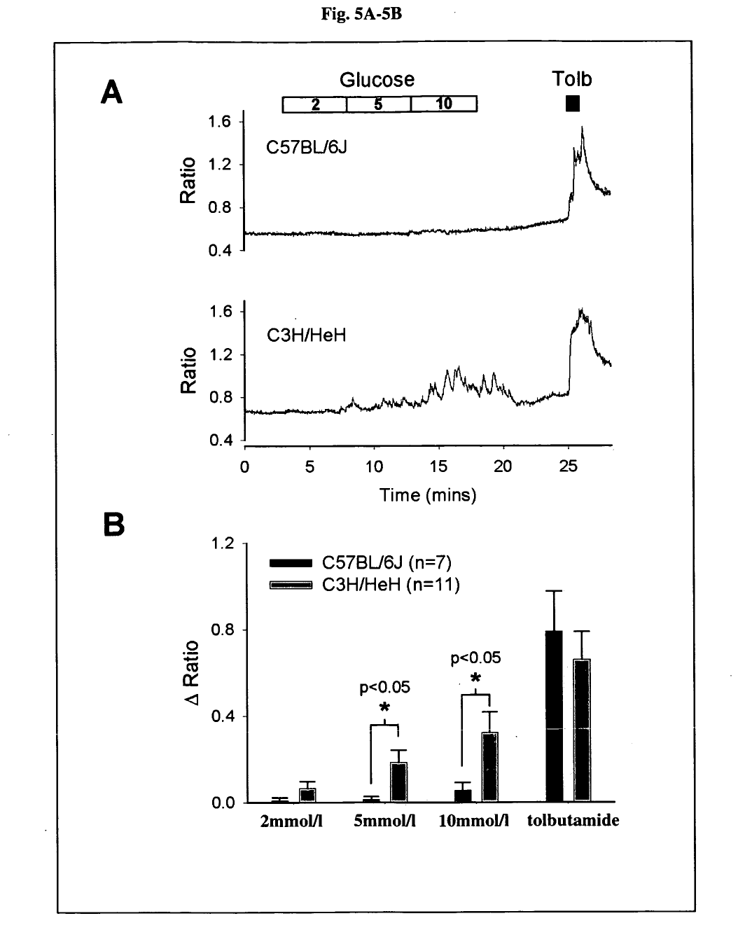

Next the effect of glucose on intracellular calcium was measured using the calcium indicator Fura 2. FIG. 5A shows representative recordings of the fluorescence ratio (F340/F380) from islet cells of both mouse strains. Although both respond to 500 μmol/l tolbutamide with a rise in [Ca2+]i, only β-cells from C3H/HeH mice show an increase in [Ca2+]i following exposure to 5 or 10 mmol/l glucose. This explains the lack of insulin secretion in response to 10 mmol/l glucose of C57BL/6J islets. The mean increase in [Ca2+]i produced by tolbutamide was not significantly different (t-test) between the two strains (FIG. 5B). In contrast, responses to 5 and 10 mmol/l glucose were significantly different (p<0.05). Furthermore, oscillations in [Ca2+]i on exposure to 10 mmol/l glucose were observed in 7 of 11 C3H/HeH β-cells, but were absent from all 7 C57BL/6J cells studied. These data indicate that the loss of insulin secretion derives from the failure of glucose to stimulate a rise in intracellular calcium. Furthermore, because tolbutamide elicits an equivalent rise in [Ca2+]i in both mouse strains, the data further suggest that glucose fails to close K_{ATP} channels and thereby elicit membrane depolarization and Ca2+ entry

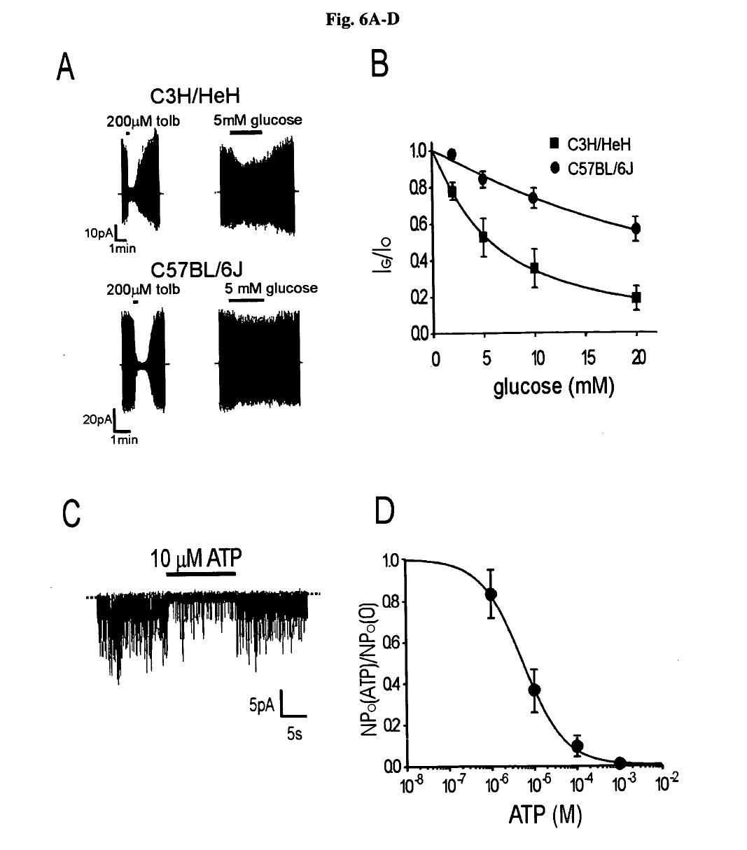

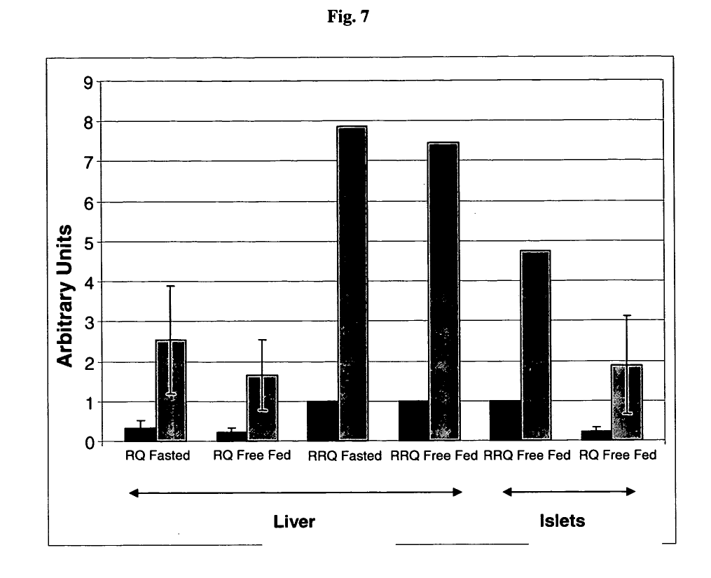

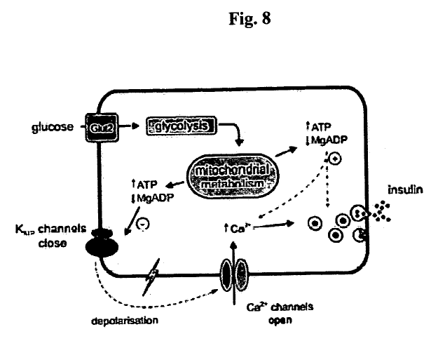

To examine this possibility, the ability of glucose to close K_{ATP} channels in C3H/HeH and C57BL/6J β-cells was studied using the perforated patch configuration to maintain metabolism intact. Strikingly, K_{ATP} currents in C57BL/6J β-cells were significantly less sensitive to glucose than those of C3H/HeH β-cells, the IC50 being 24.7±2.9 mmol/l (n=13) and 5.7±0.2 mmol/l (n=6) respectively (FIGS. 6A and B). This result is consistent with the higher fasting glucose levels and lower glucose tolerance of C57/BL6 mice. To explore if this difference in glucose sensitivity is due to differences in β-cell metabolism or in the K_{ATP} channel itself, the concentration-response curve for channel inhibition by ATP was measured. FIGS. 6 (C and D) shows that half-maximal inhibition of C57BL/6J K_{ATP} channels by ATP (IC50 10 μM) is similar to that observed for NMRI mice [Ashcroft et al., J. Physiol 416:349-367 (1989)] and for cloned K_{ATP} channels [Tucker et al., Nature 387:179-183 (1997)]. This suggests that the defect in C57BL/6J mice lies prior to K_{ATP} channel closure, probably at the level of β-cell metabolism.

iii. Candidate Gene Studies

Gluchos1 (Chromosome 13).

In a 5 cM sweep around Gluchos1 on chromosome 13 at cM 65±12 we identified several potential candidate genes: in particular, the transcription factor Isl1 (Islet gene enhancer protein 1, 113 Mbp) and Nnt (nicotinamide nucleotide transhydrogenase, 64 cM or 116 Mbp). The coding sequence of Isl1 was sequenced and no mutations found.

The coding region of Nnt was sequenced and showed two mutations. First, an exchange of T (C3H/HeH) for C (C57BL/6J) at nt104 of exon 1 that results in a missense Methionine to Threonine mutation at amino acid 35 of the protein. This mutation is located in the mitochondrial leader peptide sequence of the Nnt precursor protein [Arkblad et al., Biochim Biophys Acta 1273:203-205 (1996)]. The second mutation was found in islet and liver cDNA PCR amplified between exon 5 and 13, numbered according to the Nnt gene structure and sequence derived from 129/SvJ mice [Arkblad et al., Biochim Biophys Acta 1520:115-123 (2001)]; GENBANK AAF72982, AF257137-AF257157). The amplified fragment was ˜508 bp from C57BL/6J but ˜1261 bp from C3H/HeH cDNA. Sequencing of these products confirmed that exons 7 to 11 were completely missing in the C57BL/6J fragment, although the transcript remained in frame between exons 6 and 12 (see FIG. 7 of Toye et al., Diabetologia. 2005 April; 48(4):675-86. Epub 2005 Feb. 24). In silico analysis of cDNA database sequences from 129/SvJ, NOD, FVB, C57BL/6J and B6CBAF1 mouse strains independently confirmed that most C57BL/6 annotated transcripts also lack a 753 bp segment that encompasses the whole of exons 7 to 11 expected from the structure of the 129/SvJ gene and that of other mouse strains [Arkblad et al., Biochim Biophys Acta 1520:115-123 (2001)]. PCR amplification of exons from genomic DNA of 26 different inbred strains and sub-strains indicates that only C57BL/6J is missing exons 7 to 11 and that in the other strains all exons are present (data not shown). This is consistent with BLAST searches of the ENSEMBL C57BL/6J Mouse sequence (releases v13.30.1, May 2003 and NCBIM32_feb 2004) that revealed no homology to these exons.

Finally, the RNA expression level of Nnt was assayed by RT-PCR, by amplifying a 277 bp fragment bridging exons 5 and 6 of the gene. Gene expression in C3H/HeH compared to C57BL/6J mice was at least 7-fold higher in the liver and approximately 5-fold higher in islet RNA (FIG. 7).

Gluchos2 (Chromosome 11).

Gluchos2 lies in a region of comparative homology with a rat chromosome 14 diabetes QTL Dmo3 [Kanemoto et al., Mamm Genome 9:419-425 (1998)]. There are a number of potential candidate genes that lie under the peak of linkage, most notably CamK2B and Gck. The entire coding sequence of CamK2B was sequenced and showed no mutations capable of disrupting protein function. Given the crucial role of glucokinase in glucose homeostasis [Stoffel et al., Proc Natl Acad Sci USA, 89:7698-7702 (1992); Velho et al., Diabetologia 40:217-224 (1997); Byrne et al., J Clin Invest 93:1120-1130 (1994); Hattersley et al., Lancet 339:1307-1310 (1992); Njolstad et al., N Engl J Med 344:1588-1592 (2001); Vionnet et al., Nature 356:721-722 (1992)], in vitro glucokinase function in liver of 12 week male C57BL/6J was compared with that of C3H/HeH mice. C57BL/6J mice had significantly (p<0.01) lower glucokinase activity despite similar hexokinase activity: glucokinase activity was 16.61±2.144 (S.D.) mU/mg protein (n=5) for C57BL/6J compared to 22.73±2.09 mU/mg protein (n=4) for C3H/HeH (enzyme activity level was measured using triplicate Vmax assays). The islet promoter and entire coding region of the Gck gene in both strains were sequenced and showed one silent polymorphism in C57BL/6J—a C to G transversion at base 131 of exon 7.

Gluchos3 (Chromosome 9).

There are a large number of genes in the Gluchos3 region including several of potential interest. For example, Atp5l encodes a subunit of the mitochondrial F0 complex involved in ATP synthesis and proton transport. Given its role in ATP synthesis, and the tight coupling between ATP generation and insulin secretion, it seems a good causal candidate for Gluchos3. The islet-2 gene (a relative of islet-1) also lies on chromosome 9, but at the edge of the potential linkage.

This Example shows that there is a mutation resulting in missense (Met to Thr) mutation in the mitochondrial leader polypeptide of the Nnt precursor protein. A 5 exon deletion was also found in the C57BL/6J gene that removes 4 putative transmembrane helices and connecting linkers, and is expected to have a detrimental effect on protein function. Expression of Nnt RNA is more than 7-fold and 5-fold lower in C57BL/6J liver and pancreatic islets compared to C3H/HeH controls.

The nature of this mutation, its genomic position within the Gluchos1 locus, the mitochondrial location, and the predicted biological role of Nnt [Arkblad et al., Biochim Biophys Acta 1273:203-205 (1996); Hoek et al., Biochem J 254:1-10 (1988); Sazanov et al., FEBS Lett 344:109-16 (1994); Wollheim et al., Diabetes 51 (Suppl. 1): S37-S42 (2002)], coupled with the large difference in gene expression between the two strains, make it a strong candidate for the Gluchos1 locus causal gene. The human NNT gene maps to 5p13.1-5cen; interestingly a genetic modifier of the age of diagnosis of MODY3 (an early onset form of diabetes resulting from impaired insulin secretion) also maps to 5p15 [Kim et al., Diabetes 52:2182-6 (2003)].

In conclusion, impaired glucose tolerance in C57BL/6J mice is under the control of three main genes with several other genes having smaller effects. These studies show that a large component of glucose insensitivity results from impaired glucose-stimulated insulin release due to defective β-cell metabolism. The deletion in the Nnt gene makes it a strong causal candidate for Gluchos1 and the reduced expression of Gck suggests it could be the gene underlying Gluchos2. These studies support the conclusion that a mild reduction in glucokinase is compounded by a defect in mitochondrial metabolism that further reduces ATP production, leading to increased K_{ATP} currents and thus to the reduced insulin secretion that produces glucose intolerance. It seems possible that a similar deficiency in metabolism may contribute the impaired insulin secretion found in human Type 2 diabetes.

TABLE 4
Summary of significant quantitative trait loci linkages
in crosses involving C57BL/6J and related strains.

| Chr | cM | High allele | Trait and genetic cross | * |
|---|---|---|---|---|
| 6 | 61 | C57BL/6J | Increased plasma Insulin; F2 | [41] |
| | | | C57BL/6J and CAST/Ei. | |
| | | | Atherogenic diet. | |
| 2 | 87 | C57BL/6J | Increased 30 min IPGTT blood | [15] |
| | | | glucose; F2 C57BL/6J and | |
| | | | C3H/HeH | |
| 13 | 45-61 | C57BL/6J | As above | [15] |
| 8 | 72 | KK | Increased blood glucose in | [42] |
| | | | IPGTT; F2 C57BL/6J and | |
| | | | KK-A(y) | |
| 14 | 10.5 | C57BL/6J | Hyperinsulinaneima; F2 | [43] |
| | | | C57BL/6J (congenic, doubly | |
| | | | heterozygous for IR and IRS-1 | |
| | | | knockouts) and 129S6/SvEvTac | |
| 1 | 38 | B6 | Increased plasma insulin | [44] |
| | | | levels; F2 B6 and 129/Sv | |
| | | | carrying heterozygous | |
| | | | IR knockout. | |
| 2 | 28 | B6 | As above | [44] |
| 6 | 57 | B6 | As above | [44] |
| 10 | 48 | B6 | As above | [44] |
| 12 | 44 | 129/Sv | As above | [44] |
| 2 | 52 | BTBR | High fasting plasma insulin; | [19] |
| | | | F2 ob/ob population segregating | |
| | | | B6 and BTBR alleles | |
| 19 | 53 | BTBR | As above | [19] |
| 16 | 28 | BTBR | High fasting plasma glucose; F2 | [19] |
| | | | ob/ob population segregating B6 | |
| | | | and BTBR alleles | |
| 13 | 60 | TallyHo | Increased non-fasted plasma | [40] |
| | | (TH) | glucose; Backcross | |
| | | | (C57BL/6J × TH)F1 and TH | |
| 19 | 50 | TallyHo | Increased non-fasted plasma | [40] |
| | | (TH) | glucose; Backcross | |
| | | | (C57BL/6J × TH)F1 and TH | |
| | | | or (CASTEi × TH)F1 and TH. | |

Chr, Chromosome; cM, centimorgan; High allele, the strain contributing the allele associated with an increased phenotype value; Trait and genetic cross, the trait showing linkage and brief details of the genetic mapping cross used.
Ref: 41: Mehrabian et al., Cic. Res. 89: 112-130, 2001
Ref. 15: Goren et al., Endocrinology 145: 3307-3323, 2004
Ref. 19: Stoehr et al., Diabetes 49: 1946-1954, 2000
Ref. 42: Suto et al., Int. J. Obes. 26: 1517-1519, 2002
Ref. 43: Almind et al., Diabetes 52: 1535-1536, 2003
Ref. 44: Kido et al., Diabetes 49: 589-596, 2000
Ref. 40: Kim et al., Genomics, 74: 273-286, 2001.

### Example 2

### Nicotinamide Nucleotide Transhydrogenase: A Key Role in Insulin Secretion

The C57BL/6J mouse displays glucose intolerance and reduced insulin secretion. In Example 1, QTL mapping identified Nnt as a strong candidate gene for reduced insulin secretion in these mouse models of Type-2 diabetes. Nnt is a nuclear-encoded mitochondrial protein thought to be involved in free radical detoxification. To investigate its functional role, siRNA molecules were used to knock down Nnt in insulin-secreting MIN6 cells (a pancreatic β-cell line).

The present Example demonstrates that knock down of Nnt produced a dramatic reduction in insulin secretion and the rise in [Ca2+]i evoked by glucose, but not the sulphonylurea tolbutamide. Two ENU-induced point mutations also exist in Nnt (N68K, G745D). Nnt mutant mice were glucose intolerant and secreted less insulin during a glucose tolerance test. Isolated pancreatic islets showed impaired insulin secretion in response to glucose, but not to tolbutamide, and glucose failed to enhance ATP levels. From the data described herein it can be deduced that that Nnt mutations/deletion impair β-cell mitochondrial metabolism leading to less ATP production, and thereby enhanced K_{ATP} channel activity. Consequently, glucose-dependent β-cell electrical activity and insulin secretion are impaired.

A. Research Design and Methods

i. Animals

Husbandry.

Mice were kept in accordance with UK Home office welfare guidelines and project license restrictions under controlled light (12 hour light and 12 hour dark cycle), temperature (21±20° C.) and humidity (55±10%) conditions. They had free access to water (25 ppm chlorine) and were fed ad libitum on a commercial diet (SDS maintenance chow) containing 2.6% saturated fat.

Genetic Crosses.

The Harwell ENU-DNA archive (Quwailid et al., Mammalian Genome 15:585-591 (2004)) was screened and two ENU induced point mutations on Nnt were identified by denaturing High Performance Liquid Chromatography (dHPLC) using a Transgenomic WAVE system. These mice were rederived by IVF from frozen sperm using C3H/HeH eggs and the progeny intercrossed to produce mice homozygous for each mutation. Genomic DNA was extracted from the mouse tail tissue using a Qiagen DNeasy tissue kit. Mice were genotyped by pyrosequencing. Primers were designed from published records (Arkblad et al., Biochim Biophys Acta 1520:115-123 (2001)) using Pyrosequencing Assay Design software from Biotage. The pyrosequencing results were analysed using the Pyrosequencer PSQ HS 96A system.

Intraperitoneal Glucose Tolerance Tests.

Mice were fasted overnight, weighed and a blood sample collected by tail venipuncture under local anaesthetic (Lignocaine) using Lithium-Heparin microvette tubes (Sarstedt) to establish a baseline glucose level “T0”. They were then injected intraperitoneally with 2 g glucose (20% glucose in 0.9% NaCl)/kg body weight and blood samples taken at the designated times. For a fuller protocol, see EMPReSS (the European Mouse Phenotyping Resource for Standardised Screens designed by Eumorphia) simplified IPGTT (http://empress.har.mrc.ac.uk). Plasma glucose was measured using an Analox Glucose analyser GM9. Plasma insulin was measured using a Mercodia Ultra-sensitive Mouse ELISA kit according to the manufacturer's instructions.

Thermoregulation Studies.

Mice were weighed and then placed into a cabinet fridge (Sanyo Scientific, USA, blood bank refrigerator) at 4° C., containing cages which had been incubated in the fridge overnight to prevent variations in fridge temperature during the experiment. The mice were not given food but had free access to water during the procedure. Each mouse was housed individually for the duration of the study. The body temperature of each mouse was taken hourly over a six hour period using a rectal temperature probe (World Precision Instruments, UK). If the temperature of the mouse dropped below 31° C., the mouse was removed immediately and took no further part in the experiment.

ii Cell Studies

Cell Culture.

MIN6 cells (starting passage 60) were maintained in DMEM containing 25 mM glucose and supplemented with 15% heat-inactivated fetal bovine serum in humidified 7% CO₂, 95% air at 37° C. Cells were exposed to glucose-free extracellular solution for 15 minutes prior to measurement of insulin secretion or intracellular calcium

Islet Isolation.

Mice were killed by cervical dislocation, the pancreas removed, and islets isolated by liberase digestion and handpicking (Example 1). Isolated islets were dispersed into single cells by incubation in calcium-free Hank's solution (1 mM EGTA) and trituration in RPMI tissue culture medium containing 5.5 mM glucose (Gibco, U.K.) supplemented with 10% fetal calf serum, 100 U/ml penicillin, and 100 μg/ml streptomycin. Cells were maintained in this medium at 37° C. in a humidified atmosphere at 5% CO₂ in air, and used 1-2 days after the isolation.

siRNA.

Synthetic siRNAs were designed by Eurogentec and synthesised by both Eurogentec and VH Bio Ltd. A total of four siRNAs were used; Nonsense (NS), Glucokinase (Gck) and two Nnt siRNAs. Each siRNA had a Cy3 tag on the 5′ end of the sense strand to enable visualization of transfected cells. siRNA duplexes (5 nM) were transfected into MIN6 cells using Lipofectamine 2000 (Invitrogen) at 200 nM. Cells were cultured in DMEM (FBS-free) for a further 24 hours before experiment.

Quantitative RT-PCR.

Total RNA from siRNA-transfected MIN6 cells was extracted using RNeasy mini-kit (Qiagen). cDNA generated by Superscript II enzyme (Invitrogen) was analysed by RTPCR using a SYBR green PCR kit and an ABIPRISM 7700 Sequence detector (Perkin Elmer). To quantitate Nnt expression, it was PCR amplified from cDNA together with Gapdh (glyceraldehyde-3-phosphate dehydrogenase) as an endogenous control. All data were normalised to Gapdh expression. RT-PCR primers listed in 5′ to 3′ orientation are:

For Gapdh:

(SEQ ID NO:5)

Gapdh888F - CCTGCGACTTCAACAGCAACT
and

(SEQ ID NO:6)

Gapdh979RV - CCAGGAAATGAGCTTGACAAA
For Nnt:

(SEQ ID NO:7)

NntXn5inF - GATAGTTGGTGGTGGAGTTG
and

(SEQ ID NO:8)

NntXn6inR - GATGTCCACCTCCTTGCACT

The t-tests for significant differences between strains are based on RQ (Nnt/Gapdh) values. RRQ (or relative RQ) is the ratio of the nonsense siRNA-transfected MIN6 cells to that for Nnt siRNA transfected MIN6 cells.

Protein Gels and Western Blot Analysis.

Target-specific gene knockdown was verified using SDS-PAGE 12% gel and Western blot. Protein samples were heated for 3 minutes at 100° C. in sample buffer (final concentration of 30 mM Tris-HCl pH6.8, 5% glycerol, 0.005% bromophenol blue, 2.5% β-mercaptoethanol). Proteins were size-separated by electrophoresis alongside a “SeeBlue” (Invitrogen) protein ladder to allow molecular weights to be estimated. Proteins were transferred to Hybond-P membrane (Amersham) using a Transblot SD system (Biorad) by passing 100V across membrane/gel for 1 hour. Membranes were probed with a custom-made polyclonal Nnt antibody (raised against peptides, Eurogentec), Gck antibody and actin antibody (control) from Santa Cruz. ECL plus (Amersham) was then used according to manufacturers instructions to allow visualization of protein on ECL film, developed using the Compact X4 (Xograph).

Fura-2 Calcium Imaging.

MIN6 cells were cultured on 35mm Fluorodishes (World Precision Instruments) and incubated with 3 μM Fura-2-AM (Molecular Probes) for 40 min at 37° C. They were imaged at room temperature (20-24° C.) using an IonOptix fluorescence system (Boston, Mass.), with 340 nm and 380 nm dual excitation. The 510 nm emission ratio was collected at 1 Hz. Background subtraction was performed by measuring fluorescence from a cell-free region in the field of view. Cells were perfused continuously with extracellular solution containing (mM): 137 NaCl, 5.6 KCl, 2.6 CaCl₂, 1.2 MgCl₂, 10 HEPES (pH 7.4 with NaOH) plus glucose or tolbutamide as indicated. Only data from cells that responded to 500 μM tolbutamide were analysed.

Insulin Secretion.

MIN6 cells (1×10⁵) were replated, transfected with various siRNAs and cultured for 24 hours (as above) before being used in secretion assays. Pancreatic islets were isolated from wildtype and mutant mice and cultured overnight (as described above) before insulin secretion was assessed. Insulin secretion was measured during 1 hr static incubations in Krebs-Ringer Buffer (KRB solution; in mM: 118.5 NaCl, 2.54 CaCl₂, 1.19 KH₂PO₄, 4.74 KCl, 25 NaHCO₃, 1.19 MgSO₄, 10 HEPES, pH 7.4) containing 0, 2, or 10 mM glucose or 0.2 mM tolbutamide. Each glucose concentration was replicated 5-8 times. Samples of the supernatant were assayed for insulin. To determine total insulin content, insulin was extracted using 95:5 ethanol/acetic acid. Insulin was measured using a Mouse Insulin ELISA kit (Mercodia, Sweden).

ATP Measurements.

Batches of 6 islets were incubated at 37° C. for 1 hour in 50 μl KRB solution containing 2 mg/ml BSA and various concentrations of glucose. After this time, 25 μl of ice-cold 10% perchloric acid was added and islets were disrupted by vortexing for 10 sec. Each glucose concentration was tested in 5 separate assays and ATP was measured in duplicate in 10 μl aliquots from each assay, using a luciferin-luciferase bioluminescent assay (Sigma St. Louis, Mo., USA) and a TD20/20 luminometer (Turner Designs, Sunnyvale Calif.). Protein content was measured the DC protein assay kit (BioRad, Hercules, Calif.). ATP content was normalised to protein content to correct for differences in islet size.

B. Results

i. Cellular Studies

SiRNA.

To determine the functional role of Nnt in β-cells, siRNA molecules were used to silence Nnt in the insulin-secreting β-cell line MIN6. MIN6 cells were mock-transfected or transfected with one of four different siRNA duplexes; nonsense, glucokinase (Gck) or two different Nnt specific siRNAs. Gck was used as positive control as it is a key glycolytic enzyme and in its absence insulin secretion is abolished (Terauchi et al., J Biol Chem 270:30253-30256 (1995)). A scrambled (nonsense) siRNA was used as a negative control. Two separate Nnt siRNAs targeted to different regions of the gene were used to further corroborate the data.

Quantitative Real-Time PCR and Western Blotting.

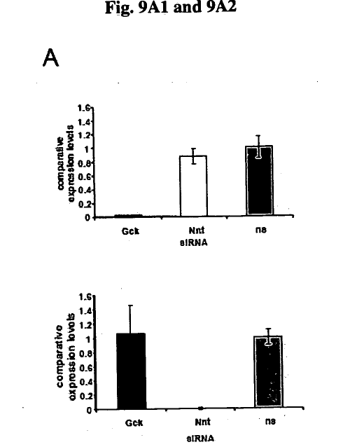

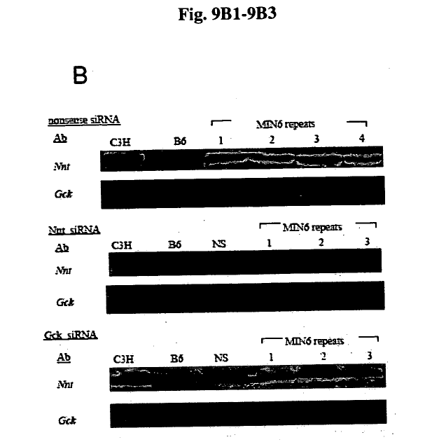

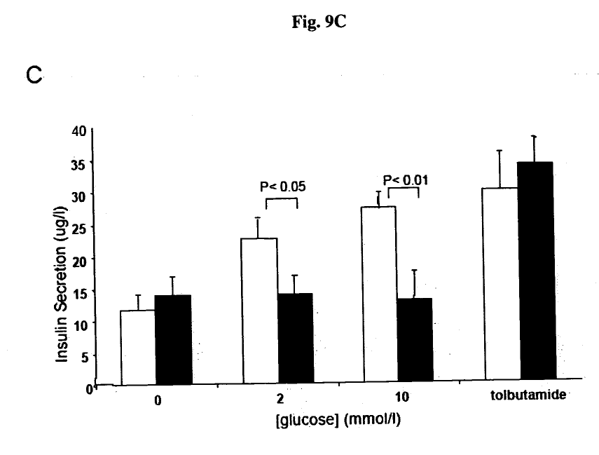

These techniques were used to verify mRNA and protein knockdown. Expression of Gck and Nnt mRNAs was abolished by siRNAs targeted against these genes, but neither was affected in cells transfected with nonsense siRNA (FIG. 9A). Similar results were found for protein expression. FIG. 9B shows Western blots of MIN6 cells probed with antibodies to Gck and Nnt following transfection with one of the three different siRNA. Pancreatic islets isolated from C3H/HeH and C57BL/6J mice are also included as positive and negative controls, respectively. The Nnt protein is not found in C57BL/6J mice although as previously reported the RNA is present (see Example 1). It is also clear that Nnt is present in MIN6 cells transfected with nonsense (upper panels in FIG. 9B) or Gck siRNA (lower panels in FIG. 9B) but not in cells transfected with Nnt siRNA (middle panels in FIG. 9B). Transfection with Gck siRNA abolished expression of Gck without altering Nnt expression (FIG. 9B, lower panels). A second Nnt siRNA also abolished protein and RNA expression (data not shown). Thus, it is demonstrated that target-specific knockdown of Nnt was achieved with both siRNAs.

Insulin Secretion studies.

Insulin secretion from MIN6 cells transfected with nonsense siRNA was compared with MIN6 cells transfected with Nnt siRNA. FIG. 9C shows that basal insulin secretion in glucose-free solution was almost identical for both nonsense and Nnt siRNA transfected cells but that glucose failed to stimulate insulin secretion from cells in which Nnt had been knocked down. However, the sulphonylurea tolbutamide (200 μM) elicited equivalent responses in both nonsense and Nnt siRNA transfected cells. Because tolbutamide closes K_{ATP} channels directly, this result suggests that the defect in insulin secretion lies prior to K_{ATP} channel closure. Under conditions used in these studies, about 95% of cells were transfected, as evidenced by their fluorescence. This explains why knockdown of Nnt was able to almost completely abolish insulin secretion in these preparations.

Calcium Imaging Studies.

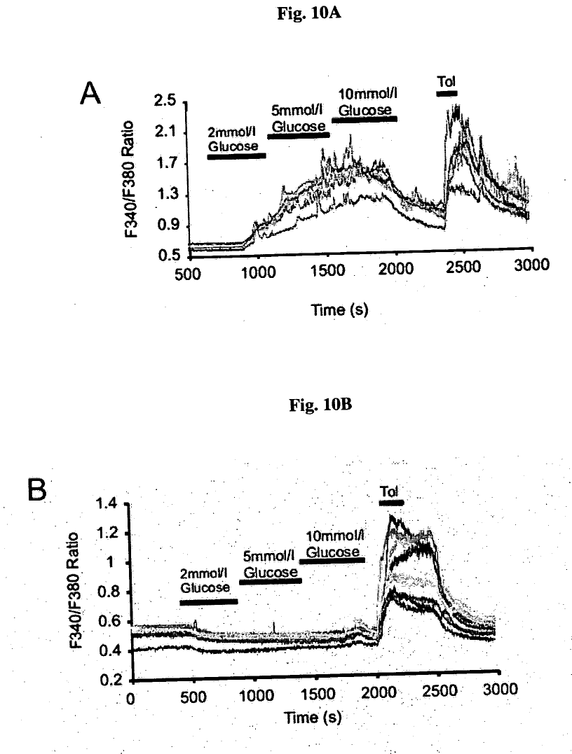

To determine the cause of impaired insulin secretion changes in the intracellular calcium concentration ([Ca]i) of MIN6 cells evoked by glucose were measured. In mock transfected cells, glucose concentrations greater than 2 mM elicited a rise in [Ca]i (FIGS. 10A, 10C). This response was unaltered by transfection with nonsense siRNA but was abolished by Gck siRNA and markedly reduced by both Nnt siRNAs (FIGS. 10B, 10C). These results resemble those found for C57BL/6J mice (see Example 1) and demonstrate that the failure of glucose to induce insulin secretion produced by downregulation of Nnt is due to the inability of glucose to promote Ca2+ influx. Tolbutamide (200 μM), however, stimulated a similar increase in [Ca]i in both cells transfected with nonsense and Nnt siRNA. (FIG. 10D). The simplest explanation of these results is that glucose fails to close K_{ATP} channels when Nnt is down regulated.

ii. Whole Animal Studies

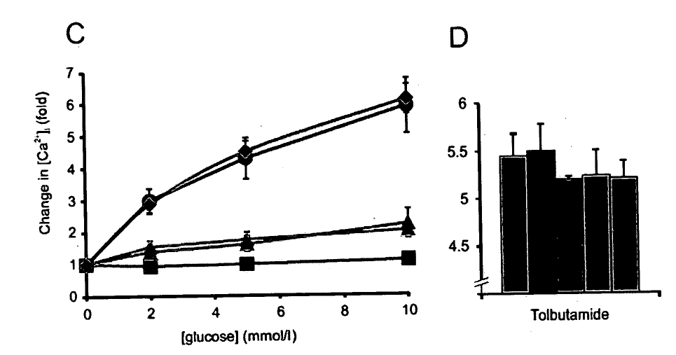

Nnt mutant mice are glucose intolerant and have reduced insulin secretion. Two mutant alleles of Nnt were identified by screening the Harwell ENU-DNA archive (Quwailid et al., Mammalian Genome, 15:585-591 (2004)). The first (N68K) was an exchange of C for A (nt 204) in exon 2 that results in a nonconservative missense asparagine to lysine mutation at amino acid 68 of the protein. This mutation is located in the predicted NAD-binding domain of Nnt. The second mutation (G745D) was found in the membrane spanning domain of Nnt and consisted of an exchange of G for A (nt 2224) in exon 14, resulting in a nonconservative substitution of glycine by aspartic acid at amino acid 745. Both residues N68 and G745 are highly conserved across species (FIG. 11A).

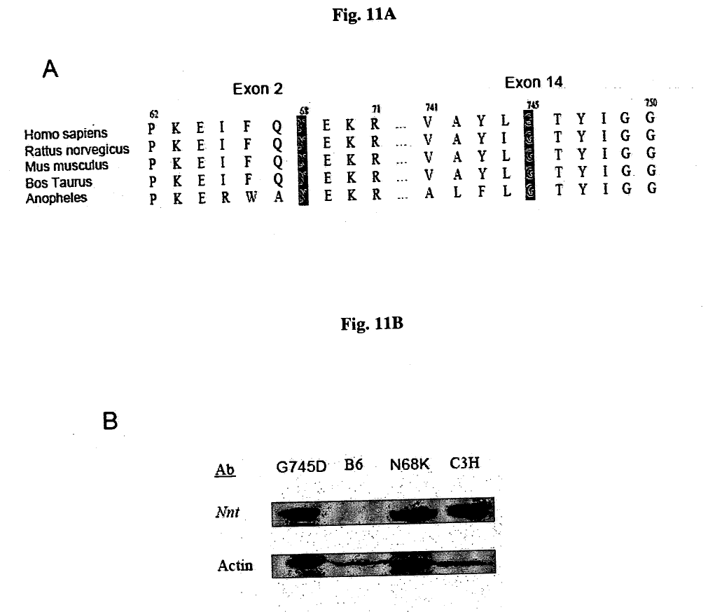

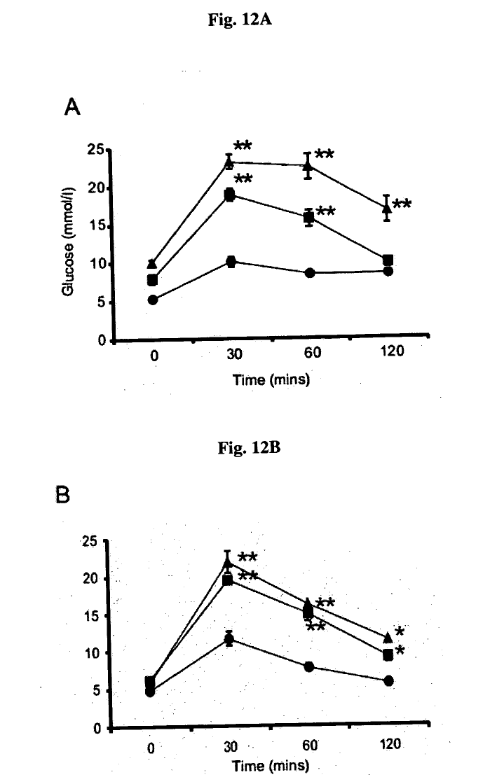

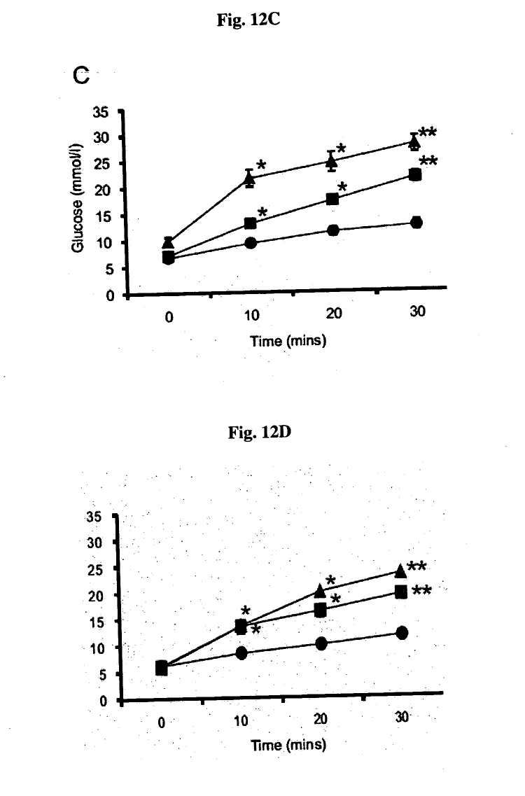

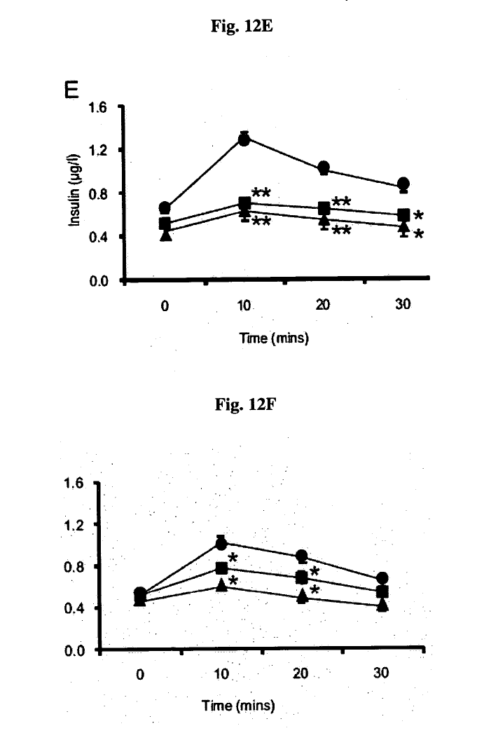

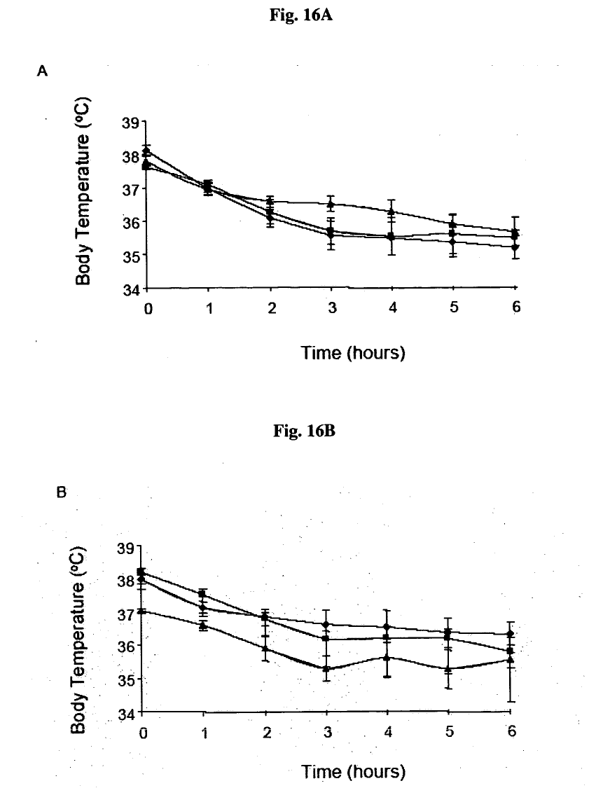

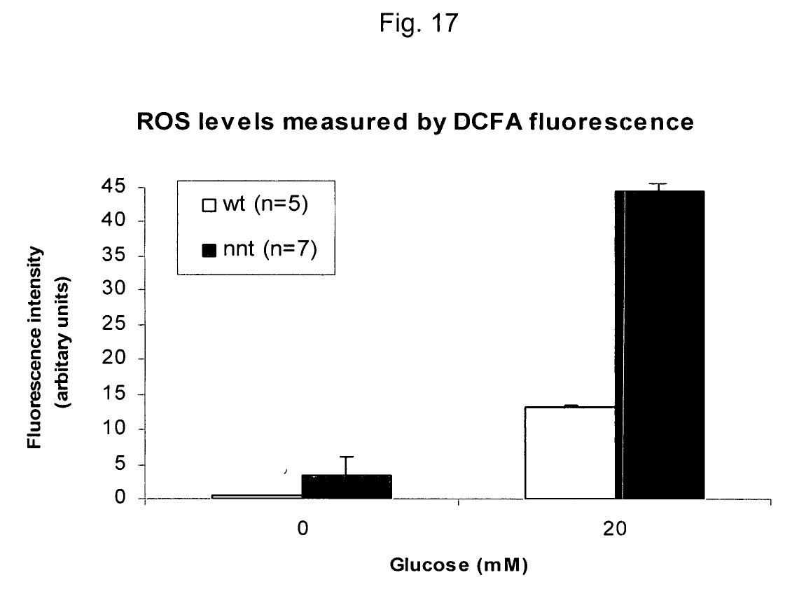

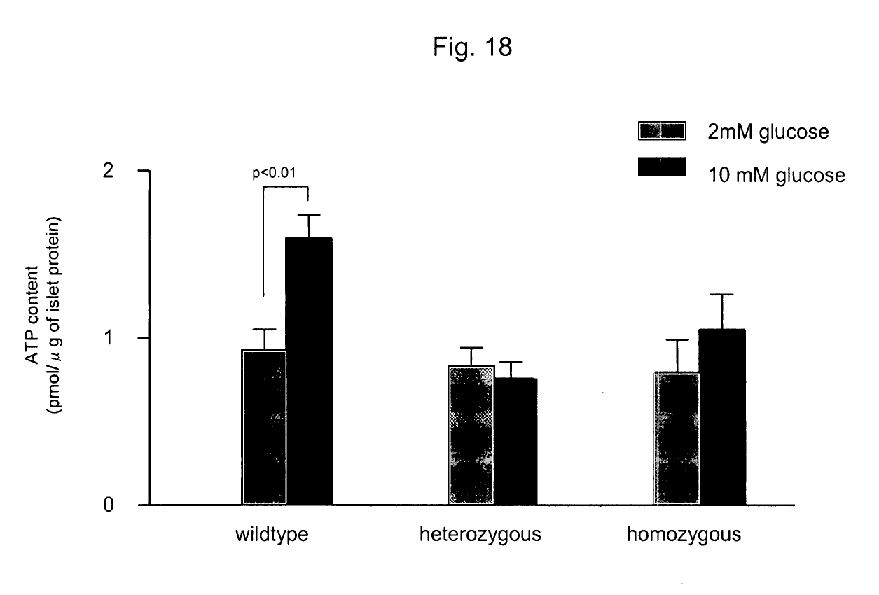

Both mutants were recovered as live mice by IVF (male F1 BALB/c×C3H/HeH sperm and C3H/HeH eggs), and the progeny were intercrossed to produce mice homozygous for the mutations. Intraperitoneal glucose tolerance tests (IPGTT) revealed that mice heterozygous and homozygous for the N68K mutation were markedly less glucose tolerant than their wild-type littermates (FIG. 12A). During a 2 hour IPGTT, blood glucose levels rose significantly higher and took longer to return to baseline in homozygous 12 week old male mice. Furthermore, heterozygotes were not as glucose intolerant as homozygotes (FIG. 12A). These differences in glucose tolerance persisted at 16 weeks (FIG. 12C). Both heterozygous and homozygous N68K mice also secreted substantially less insulin over a 30 minute period following a glucose challenge (FIG. 12E). Very similar results were obtained for female mice (see FIGS. 16 and 17).

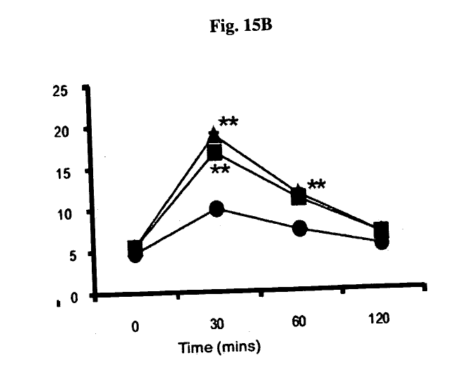

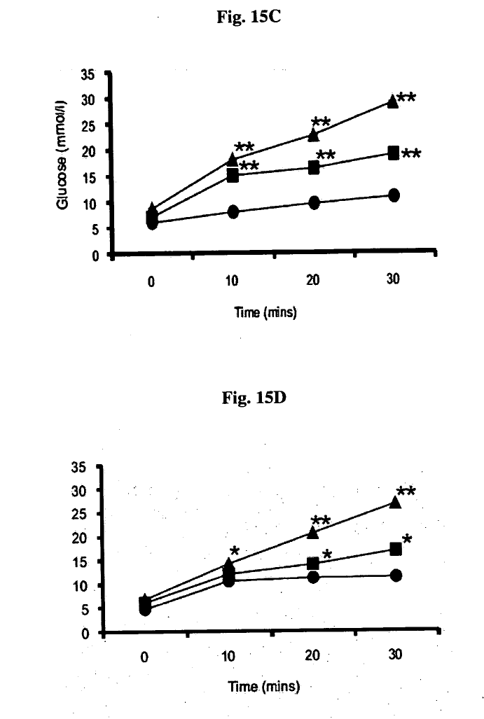

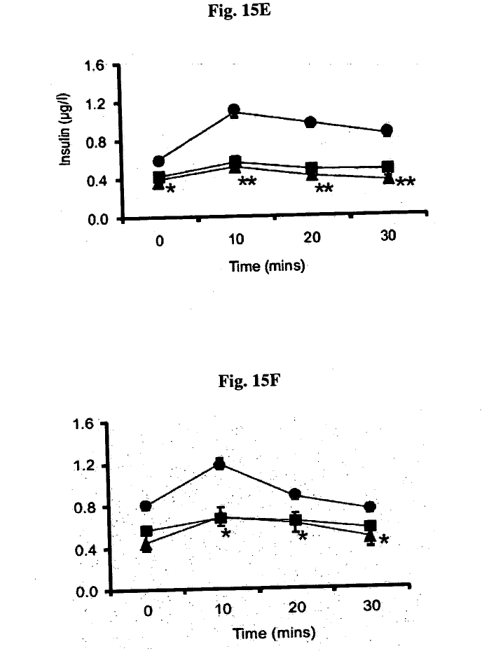

FIG. 12B shows that 12-week old male mice heterozygous and homozygous for the G745D mutation were also significantly less glucose tolerant than controls during a 2 hour IPGTT. Interestingly, the glucose tolerance profiles over 2 hours are very similar for heterozygous and homozygous mice suggesting that the G745D mutation affects heterozygous mice as severely as homozygous mice. Insulin secretion and glucose tolerance over 30 minutes were also impaired in 16 week old mice (FIGS. 12D, 12F). Similar results were found for G745D mutant female mice (see FIGS. 15 and 16).

Thus, two separate mutations in the Nnt gene confer impaired glucose tolerance and insulin responses. The ability of Nnt mutant mice to thermoregulate was compared with that of wild-type mice by measuring core temperature during an extended period at 4° C. All mice showed a slow and very slight drop in temperature over time, but no difference was observed between wild-type, heterozygous and homozygous mice for either the N68K or G745D mutations (see FIGS. 15 and 16). These data indicate that the Nnt mutations do not uncouple mitochondrial respiration in brown adipose tissue (or muscle) to an extent that influences non-shivering thermogenesis.

Nnt Expression in Nnt Mice.

Western blotting was used to determine if Nnt mutant mice express the Nnt protein. FIG. 11B shows that Nnt was expressed in pancreatic islets of homozygous N68K and G745D Nnt mice: therefore, these mutations must affect protein function and/or mitochondrial membrane targeting.

Insulin Secretion Studies in Nnt Mice.

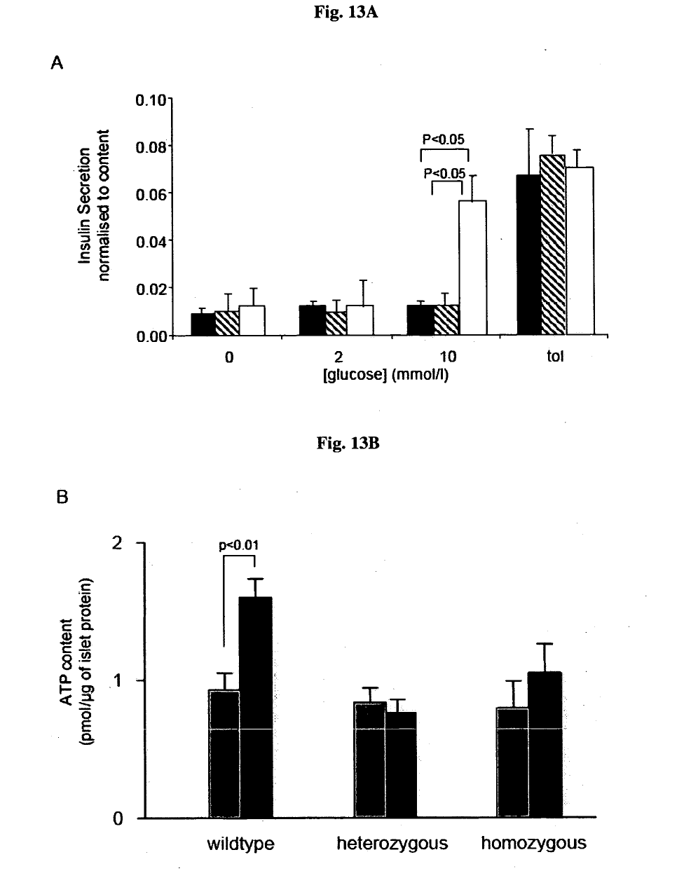

Insulin secretion from pancreatic islets isolated from Nnt mutant mice was measured. Insulin secretion was normalized to insulin content to control for any differences in islet size. There was no significant difference in insulin content between wild-type or mutant islets. Background secretion at 0 or 2 mM glucose from homozygous or heterozygous G745D islets was not significantly different from that of wild-type islets, but glucose (10 mM) stimulated insulin secretion was substantially reduced. However, the ability of tolbutamide to stimulate secretion was similar in all three types of mice (FIG. 13A). Similar results were obtained for islets isolated from heterozygous or homozygous N68K mice (data not shown). These results resemble those found for MIN6 cells and suggest that both Nnt mutations produce a loss of protein function.

ATP Measurements.

The data reported here, and those found for C57BL/6J mice (see Example 1), are consistent with the idea that Nnt deletion or mutation impairs β-cell metabolism and thereby insulin secretion. Therefore ATP concentration in pancreatic islets isolated from wild-type, heterozygous and homozygous Nnt mutant mice was measured. As FIG. 13B shows, ATP content increased significantly (1.8-fold; P<0.05) in wild-type islets when glucose was increased from 2 to 10 mM. However, no increase was detected in either heterozygous or homozygous G745D Nnt islets (FIG. 13B). No significant difference was observed in resting ATP content (at 2 mM glucose). Similar results were obtained for G745D islets (data not shown).

C. Discussion of Results

The results described in this Example demonstrate that Nnt plays an important role in the regulation of insulin secretion from the pancreatic β-cell. Mice in which this protein is deleted (e.g. C57BL/6J) or mutated (Nnt-N68K and Nnt-G745D), are characterised by impaired glucose tolerance. This is a consequence of impaired ATP production in response to glucose elevation, which translates into a marked decrease in insulin secretion.

Furthermore, siRNA silencing of Nnt in insulin-secreting MIN6 cells abolishes the glucose-dependent increase in both [Ca]i and insulin secretion. However, both are elicited when the sulphonylurea tolbutamide is used to close K_{ATP} channels. These results are consistent with the idea that metabolic regulation of K_{ATP} channel activity is impaired in Nnt mutant mice, or following Nnt knock down in the insulin-secreting MIN6 cell line. They are also in consistent with the location of Nnt in the inner mitochondrial membrane.

Role of Nnt.

Several studies have suggested that Nnt is involved in detoxification of reactive oxygen species (ROS) generated by the electron transport chain (Hoek et al., Biochem J 254:1-10 (1988)). Superoxide is produced by electron leakage during mitochondrial metabolism primarily at Complexes I and III (Raha et al., Trends Biochem Sci 25:502-508 (2000)). In highly metabolically active tissues up to 5% of the O₂ consumed is converted to superoxide. A significant fraction (at least 50%) of the superoxide generated is released into the mitochondrial matrix (Muller et al., J Biol Chem 279:49064-49073 (2004)) and as it is membrane impermeant must be detoxified within the mitochondrion itself. This is achieved by conversion to H₂O₂ by manganese-dependent superoxide dismutase 2 (SOD2) and subsequent oxidation to water by the glutathione cycle (FIG. 14). This process requires a continuous supply of NADPH. In mitochondria, this is provided by nicotinamide nucleotide transhydrogenase, which converts NADP and NADH into NADPH and NAD. Thus, in the absence of Nnt, one might expect an increase in the concentration of H₂O₂.

Previous studies have shown that H₂O₂ inhibits insulin secretion in rodent β-cells by activating the K_{ATP} channel currents and hyperpolarising the plasma membrane (Krippeit-Drews et al., J Physiol 514 (Pt 2) 471-481 (1999)). Consistent with these findings, tolbutamide was able to stimulate insulin secretion from pancreatic islets treated with H₂O₂. Opening of K_{ATP} channels by H₂O₂ has also been observed in ventricular myocytes (Goldhaber et al., J Physiol 477 (Pt 1): 135-147 (1994)) and substantia nigra neurones (Avshalumov et al., Proc Natl Acad Sci USA 100:11729-11734 (2003)). However, H₂O₂ does not activate the K_{ATP} channel directly, as it is ineffective in excised membrane patches or in whole-cell recording configurations that do not preserve β-cell metabolism (Nakazaki et al., Diabetes 44:878-883 (1995)). Rather, H₂O₂ mediates its effect on K_{ATP} channel activity by reducing cellular ATP production (Maechler et al., Nature 414:807-812 (1999)); Krippeit-Drews et al., J Physiol 514 (Pt 2):471-481 (1999)). The reduction in glucose-dependent ATP generation observed in Nnt mutant mice is consistent with the hypothesis that loss-of-function mutations in Nnt lead to elevation of H₂O₂.

Because H₂O₂ depolarises the mitochondrial membrane potential in β-cells (Maechler et al., J Biol Chem 274:27905-27913 (1999)), this may explain how it compromises ATP production. Since more than 95% of ATP in the β-cell is produced by the mitochondria, and mitochondrially generated ATP is of primary importance for insulin release (Silva et al., Nat Genet 26:336-340 (2000)); Maechler et al. Nature 414:807-812 (2001)), small changes in H₂O₂ resulting from impaired ROS detoxification by Nnt might have a significant effect on insulin secretion. This idea is consistent with the fact that male (but not female) C57BL/6J mice fail to upregulate antioxidant enzymes in pancreatic islets in response to oxidative stress and show more severely impaired glucose tolerance than female mice (Friesen et al., Diabetologia 47:676-685 (2004)).

The mechanism by which H₂O₂ influences mitochondrial membrane potential, and thereby ATP production, is unknown. However, one possibility is that it involves activation of uncoupling protein 2 (UCP2) (Echtay et al., Nature 415:96-99 (2002)); Brand et al., Free Radiac Biol Med 37:755-767 (2004)). Reactive oxygen species, such as superoxide, enhance UCP2 activity (Krauss et al., J Clin Invest 112:1831-1842 (2003); Brand et al., Free Radic Biol Med 37:755-767 (2004)). This leads to enhanced proton leakage across the inner mitochondrial membrane, which reduces the electromotive force and thereby ATP production. Consequently, enhanced UCP2 activity impairs insulin secretion (Krauss et al., J Clin Invest 112:1831-1842 (2003); Chan et al., Diabetes 50:1302-1310 (2001)) and reduced UCP2 activity increases insulin release (Zhang et al., Cell 105:745-755 (2001)). We therefore propose that knockdown of Nnt results in an increased production of H₂O₂, which leads to activation of UCP2, and thereby reduces ATP production (FIG. 14). In turn, this leads to increased K_{ATP} channel activity and a membrane hyperpolarization that prevents insulin secretion.

The deleterious effects of mitochondrially derived superoxide are illustrated by the fact that mice lacking the mitochondrial matrix superoxide dismutase (SOD2) have higher levels of mitochondrial ROS and die within a few days of birth (Li et al., Nat Genet 11:376-381 (1995)). Of particular interest is that survival time is highly correlated with the genetic background of the mutant animals. Thus, SOD2 knockout mice on the DBA/2J background survive for up to 8 days after birth, whereas on the C57BL/6J background, most SOD2 knockout mice die at embryonic day 15 and any that do survive to term die within 24 hours of birth. Mapping of genetic modifiers have suggested that Nnt is primarily responsible for these differences in survival time (Huang, T. T., Naeemuddin, M., and Epstein, C. J.; http://www.complextrait.org/archive/2004/PDF/huang_abstract.pdf). These findings are consistent with the idea that Nnt is involved in ROS detoxification. Further support comes from the fact that a C. elegans mutant lacking functional Nnt displays increased sensitivity to oxidative stress (Arkblad et al., Free Radic Biol Med 38:1518-1525 (2005)).

It has also been suggested that Nnt may exert a fine control of flux through the tricarboxylic acid cycle, by regulating the activity of isocitrate dehydrogenase (Sazanov et al. FEBS Lett 344:109-116 (1994)). However, this mechanism is unlikely to be very important in pancreatic β-cells, because the ATP required for K_{ATP} channel closure is generated primarily from NADPH derived from glycolysis and when both mitochondrial NADH shuttle systems are ablated K_{ATP} channel closure and insulin secretion are inhibited (Dukes et al., J Biol Chem 269:10979-10982 (1994); Eto et al., J Biol Chem 274:25386-25392 (1999a); Eto et al., Science 283:981-985 (1999b).

Nnt ENU Mutant Mice.

Both mutant mouse models of Nnt were significantly glucose intolerant and had impaired insulin secretion. In this respect, the phenotype was similar to that found for C57BL/6J mice, which have a 5 exon deletion in Nnt (see Example 1). ENU mutagenesis introduces multiple mutations in the genome and with a per locus mutation rate of 0.00108 and 30,000 genes, there will be an estimated 32 dominant functional mutations per F1 founder mouse (Quwailid et al., Mammalian Genome 15:585-591 (2004); Coghill et al., Nat Genet 30:255-256 (2002)). These mutations are segregated by subsequent backcrossing and, ultimately, intercrossing. The two ENU Nnt mutations we identified were independently derived and are thus extremely unlikely to share any ENU-induced mutations in genes other than Nnt. Thus, the observed phenotypes are due to the Nnt mutations carried by the two lines.

Both homozygous Nnt mutations appear to have a greater effect on glucose tolerance than that observed for C57BL/6J mice, yet QTL mapping suggested that Nnt contributed <10% of the glucose intolerant phenotype of C57BL/6J mice (see Example 1). The ENU mutations are on a BALB/c background and have been backcrossed to C3H/HeH and then intercrossed to produce homozygous animals. In contrast, the QTL mapping was performed between C57BL/6J and C3H/HeH in an F2 intercross. Thus the genetic composition of the mice is different. The smaller variance seen in the QTL mapping studies could be explained if C57BL/6J mice possessed compensatory mechanisms that reduced the severity of the Nnt deletion. The effect of genetic background on phenotype is well established: one example is the ob/ob mutation that shows large differences in diabetes susceptibility on BTBR and C57BL/6J genetic backgrounds (Stoehr et al., Diabetes 49:1946-1954 (2000)).

QTL mapping may also underestimate the contribution of an individual gene to an individual quantitative trait because the effect is estimated in a genetically heterogeneous F2 population using linked genetic markers. Furthermore, in an F2 population, new gene interactions may be unmasked (Stoehr et al., Diabetes 49:1946-1954 (2000)). Thus, the phenotype of the F2 generation may differ from that observed on the original (or indeed other) genetic backgrounds. This may explain why Nnt and C57BL/6J mice have a similar phenotype despite QTL mapping suggesting Nnt is only a minor contributor to the C57BL/6J phenotype.

There was no significant difference in phenotype between the heterozygous and homozygous Nnt mice for the G745D mutation. Likewise, there was no difference in ATP generation or insulin secretion between heterozygous and homozygous animals.

Because NNT functions as a homodimer (Hoek et al., Biochem J 254:1-10 (1988), this might be due to a dominant negative effect in which the presence of a mutant subunit in the homodimer impairs the function of the other subunit, thus inactivating the whole dimer. Alternatively it may be due to a gene dosage effect (i.e. >50% of functional protein is required to produce the wild-type phenotype).

Why is the Defect Only Seen in β-cells?.

Although Nnt is widely expressed in human tissues (Arkblad et al., Comp Biochem Physiol B Biochem Mol Biol 133:13-21 (2002)), it appears that only the β-cell is particularly sensitive to mutations and deletion of the Nnt gene as mice carrying Nnt mutations do not suffer from any obvious problems other than glucose intolerance. Strikingly the C57BL/6J strain has been widely used for decades without the deficiency in Nnt being phenotypically obvious. The lack of an extra-pancreatic phenotype is not because of redundancy, since there is only one copy of the gene in the genome and no other protein has been proposed to serve a similar role. A possible explanation may be that the β-cell is particularly sensitive to small changes in cytosolic ATP, perhaps because its metabolism has evolved to be very sensitive to blood glucose levels. Furthermore, the β-cell resting potential is largely determined by the activity of the K_{ATP} channel (Ashcroft et al., Prog Biophys Mol Biol 54:87-143 (1989); Ashcroft et al., Hum Mol Genet, 13 Spec No 1: R21-31 (2004)). Small changes in K_{ATP} currents may have marked effects on insulin secretion, as evidenced by the effects of mutations in Kir6.2, the pore-forming subunit of this channel (Sakura et al., FEBS Lett 377:338-344 (1995)) in both human patients with neonatal diabetes (Gloyn et al., N Engl J Med 350:1838-1849 (2004); Proks et al., Proc Natl Acad Sci USA 101:17539-17544 (2004)) and in transgenic mice (Koster et al., Cell 100:645-654 (2000)). Although Kir6.2 is expressed in multiple tissues (Sakura et al., FEBS Lett 377:338-344 (1995)), except for the most severe mutations, no functional effects are manifest in tissues other than the β-cell. Thus the effects of small changes in ATP production (rather than ATP sensitivity) produced by impaired Nnt function may be largely confined to the β-cell.

It is also worth noting that there was no difference in resting ATP content between pancreatic islets of wild-type, heterozygous and homozygous Nnt mice (FIG. 13B), and that the Nnt mutation only compromises glucose-dependent ATP generation. This was also true for insulin secretion (FIG. 13A). Both observations may be related to the fact that the rate of ROS generation is enhanced when metabolism increases (Fridlyand et al., Diabetes 53:1942-1948 (2004)).

Although, in β-cells, glucose does not elevate H₂O₂ levels, this may be due to a compensatory increase in NADPH (Martens et al., J Biol Chem 280:20389-20396 (2005)). In Nnt mice, however this compensation would not be expected to occur, so that ROS would increase with glucose.

Physiological Relevance for Type 2 Diabetes.

Compared to control pancreatic islets, type-2 diabetic islets have reduced glucose oxidation and insulin secretion (Del Guerra et al., Diabetes 54:727-735 (2005)). This may be partially a consequence of reduced mRNA expression of GLUT1, GLUT2 and glucokinase. However, an increase in markers of oxidative stress, which correlates with the degree of impairment in glucose stimulated insulin release, has also been observed. Interestingly, following 24-hour culture in the presence of 30 μM glutathione, the secretory response of type 2 diabetic islets to glucose improved significantly (Del Guerra et al., Diabetes 54:727-735 (2005)). Thus an underlying metabolic defect, resulting in increased oxidative stress, contributes to impaired insulin secretion in human type-2 diabetic patients (see also Green et al., Diabetes 53 Suppl 1: S110-118 (2004)). Given the effects of impaired Nnt function in mice, it would clearly now be worth screening human patients to determine if polymorphisms in the Nnt gene are associated with type 2 diabetes. Furthermore, the key role of Nnt in ROS detoxification, and the fact that insulin secretion is abolished when this gene is mutated or deleted, suggests that drugs which enhance Nnt function or expression might provide a valuable new strategy for treating type 2 diabetes.

### Example 3

### Transgenic Rescue Experiments to Further Verify Role of Defective Nnt in Glucose Intolerance and Impaired Insulin Secretion

In further studies to corroborate the findings described herein, the inventors rescued the Nnt deletion in C57BL/6J mice by transgenic expression of the entire Nnt gene sequence contained within a bacterial artificial chromosome (BAC). This BAC transgenic line was prepared using BAC RP22-455H18 derived from a 129S6/SvEvTac mouse BAC library obtained from Children's Hospital Oakland Research Institute. The BAC containing all 21 exons of the Nnt gene, as well as considerable 5′ and 3′ flanking intergenic DNA, was microinjected into the pronucleus of one-cell C57BL/6J embryos. Mice born were genotyped for the missing Nnt exons from tail tip DNA and offspring testing positive for the BAC were backcrossed to C57BL/6J. All mice were phenotyped at 12 and 16 weeks of age by IPGTT for plasma glucose and insulin responses.

Experimental Methods

Briefly, transgenic mice were generated by pronuclear injection of closed circular BAC DNA into mouse C57BL/6J one cell embryos. 3-week-old C57BL/6J were superovulated with 5 units PMS, followed 46 hours later by 5 units hCG and mated with C57BL/6J males overnight. The next day, females with copulation plugs were culled, and embryos harvested and treated with hyaluronidase to remove cumulus cells⁸. DNA prepared using a Qiagen Large Construct kit was resuspended in polyamine buffer (10 mM Tris-HCl pH 7.5, 0.1 mM EDTA pH 8.0, 100 mM NaCl, 30 μM spermine, 70 μM spermidine) immediately prior to microinjection. Between 100 and 200 embryos were injected with BAC DNA at a concentration of 1.0 ng/ul. Injected embryos were transferred on the same day to the oviducts of pseudopregnant CD1 recipient females. Litters arising from these transfers were genotyped at 3 weeks of age using ear biopsies to identify transgenic animals.

Genomic DNA was extracted from mouse tail tissue using a Qiagen DNeasy tissue kit. Mice were genotyped by PCR of missing exons 7 and 11 of the Nnt gene. Primers listed in 5′ to 3′ orientation are: for Nnt exon 7: NntXn7-Forward gtgcattgaaccctcaaagg (SEQ ID NO:9) and NntXn7-Reverse caggtaagaaagctcctgtttt (SEQ ID NO:10); for Nnt exon 11: NntXn11-Forward tcctgctattcctcctcctg (SEQ ID NO:11) and NntXn11-Reverse gctgccttgactttggatatt (SEQ ID NO: 12). PCR products were visualized on a 2% agarose gel run at 200V for 25 minutes.

Total RNA from mouse liver was extracted using an RNeasy mini-kit (Qiagen). cDNA generated by Superscript II enzyme (Invitrogen) underwent PCR amplification across the deleted fragment of Nnt and several introns. Primers listed in 5′ to 3′ orientation are: for Nnt fragment 1 Forward: cagcacagctctgattccag (SEQ ID NO:13) and Nnt fragment 1 Reverse: gtcaaaccgaagaccgtggctgag (SEQ ID NO:14); for Nnt fragment 2 Forward: gtgatgaaggatggcaaagtg (SEQ ID NO:15) and Nnt fragment 2 Reverse: gcaggtggttttctggtgactc (SEQ ID NO:16).

Intraperitoneal glucose tolerance tests were performed substantially as described throughout the specification. Briefly, mice were fasted overnight, weighed and a blood sample collected by tail venipuncture under local anaesthetic (Lignocaine) using Lithium-Heparin microvette tubes (Sarstedt) to establish a baseline glucose level “T0”. They were then injected intraperitoneally with 2 g glucose (20% glucose in 0.9% NaCl)/kg body weight and blood samples taken at the designated times. For a fuller protocol, see EMPReSS (the European Mouse Phenotyping Resource for Standardised Screens designed by Eumorphia) simplified IPGTT. Plasma glucose was measured using an Analox Glucose analyzer GM9 (Analox, London, UK). Plasma insulin was measured using a Mercodia Ultra-sensitive Mouse ELISA kit (Mercodia, Sweden) according to the manufacturer's instructions.

The pancreatic islets were isolated as described above.

Nnt expression was verified using SDS-PAGE 12% gel and Western blot. Protein samples were heated for 3 minutes at 100° C. in sample buffer (final concentration of 30 mM Tris-HCl pH6.8, 5% glycerol, 0.005% bromophenol blue, 2.5% β-mercaptoethanol). Proteins were size-separated by electrophoresis alongside a “SeeBlue” (Invitrogen, UK) protein ladder to allow molecular weights to be estimated.

Proteins were transferred to Hybond-P membrane (Amersham, UK) using a Transblot SD system (Biorad) by passing 100V across membrane/gel for 1 hour. Membranes were probed with a custom-made polyclonal Nnt antibody raised against amino acids 1073-1086 at the C-terminus of the peptide (Eurogentec, Belgium) antibody also was raised against amino acids 1008-1023, and actin antibody (control) from Santa Cruz. ECL Plus (Amersham, UK) was then used according to manufacturers' instructions to allow visualization of protein on ECL film, developed using the Compact X4 (Xograph).

Results

Two independent transgenic lines B6-TG1 and B6-TG2 were obtained. BAC complementation was verified first by PCR of the BAC vector itself, and secondly at the transcriptional level by detection of cDNA missing exons 7 through to 11 in the Nnt gene. In the PCR of BAC vector pBACe3.6 (U80929) using three primer sets of equal product size targeted to the start, middle and end of the vector sequence, all three bands were clearly present in the C57BL/6J mice carrying the entire Nnt gene sequence (B6-TG1 and B6-TG2) whereas in the C57BL/6J littermates without the transgene (B6-TG negative controls) there was no amplification. This confirmed that the presence or absence of missing Nnt exons is due solely to the presence of the BAC. For the RT-PCR analysis of Nnt cDNA expression across missing exons 7-11 endogenous to the C57BL/6J mouse it was seen that the naturally occurring multi-exon deletion in the C57BL/6J strain is too large, even at the cDNA level, to be amplified easily in a single PCR reaction. Therefore, it was amplified as two fragments both spanning several intronic regions. Hence two bands are visible for the C57BL/6J expressing the BAC (B6-TG1 and B6-TG2) and both bands are absent in their littermate controls (B6-TG1 negative and B6-TG2 negative). C3H/HeH was used as a positive control, as C3H/HeH mice express all 21 exons of the Nnt gene. Wildtype C57BL/6J and deionised water were run as negative controls.

The above results also were observed at the protein level by Western blotting. The amount of Nnt protein produced by both transgenic lines was similar to, or higher than, that produced by a C3H/HeH control strain that harbours wildtype Nnt. Nnt protein was completely absent in B6-TG negative controls and stock C57BL/6J mice. Both the transgenes were transmitted through the germline and lines were maintained by crossing to wild type C57BL/6J mice.

In both lines, expression of wild type Nnt on the C57BL/6J background in male mice induced improvements in glucose tolerance following a glucose challenge at 12 and 16 weeks of age compared to non-transgenic littermates. The inventors therefore looked for differences in insulin secretion by carrying out an IPGTT taking samples at 0, 10, 20 and 30 minutes after glucose injection. Insulin secretion by B6-TG1 and B6-TG2 mice was significantly higher (for the same glucose challenge per gram bodyweight) than for B6-TG negative mice. No difference in body weight was observed between the transgenic lines compared with the C57BL/6J littermates without the transgene. Similar results were seen in female C57BL/6J mice although glucose intolerance in females is less pronounced (Toye, et al. Diabetologia 48, 675-86, 2005).

To further confirm that a defect in beta-cell function, due to a deletion in Nnt, underlies the glucose intolerance of C57BL/6J mice and that this is ameliorated in the transgenic rescue mice, the ability of glucose to stimulate insulin secretion from pancreatic islets isolated from B6-TG and B6-TG-negative mice was compared. This showed that there was no difference in basal insulin secretion (at 0 and 2 mM glucose) between the two mouse strains, but that insulin secretion in response to 20 mM glucose was increased in B6-TG islets in comparison to B6-TG negative islets (p<0.05). The K_{ATP} channel blocker tolbutamide stimulated secretion comparably in both strains, as expected because it bypasses the steps in metabolism-secretion coupling that involve Nnt (Freeman, et al., Cell Metab 3, 35-45, 2006; Arkblad et al. Free Radic Biol Med 38, 1518-25, 2005; Hoek & Rydstrom, Biochem J 254, 1-10, 1988). There was clear evidence that B6-TG mice have normal glucose tolerance resembling that of C3H/HeH control mice, whereas both B6-TG negative and C57BL/6J mice display significantly higher plasma glucose levels after 60 and 120 minutes following IPGTT.

Of the 23 mouse strains previously screened⁵, including six other C57BL mouse strains, only C57BL/6J had the naturally occurring deletion in Nnt.

This example again shows that the deletion of Nnt observed in C57BL/6J mice accounts for glucose insensitivity and reduced insulin secretion in these mice. This supports the concept that primary genetic defects in beta-cell metabolism contribute to the pathogenesis of impaired glucose homeostasis. Previous studies (Kayo et al. Comp Med 50, 296-302, 2000) suggested that this locus would account for approximately 10% of the variance of the phenotype. However, this is in the F₂ genetic environment rather than a pure C57BL/6J genetic environment where Nnt alone appears to be sufficient to account for the phenotype. Thus the genetic compositions of the mice are different and this illustrates the importance of genetic interactions in determining quantitative traits.

### Example 4

### Role of Glucose Metabolism in Oxidative Stress in β-cells

In addition, it is contemplated that Nnt plays a role in detoxification of free radicals/reactive oxygen species, not only in diabetes but also in other conditions caused by oxidative stress (e.g., motor neurone disease, ageing). The present Example provides evidence that down-regulation of Nnt results in the increase of free radicals in the cell via regulation of UCP2. More particularly, the inventors have observed that glucose metabolism in β-cells leads to elevation of free radicals, such as H₂O₂. In mice carrying mutations/deletions in the NNT gene, detoxification of ROS via NADPH-dependent mechanisms will be impaired and the level of ROS will be increased relative to wild-type mice. FIG. 17 shows that ROS increase more in β-cells isolated from NNT mutant mice than wild-type mice, at 20 mM glucose.

The result of an increase in ROS will be activation of uncoupling protein 2 (UCP2). This will result in a decrease in mitochondrial membrane potential and lower production of ATP.

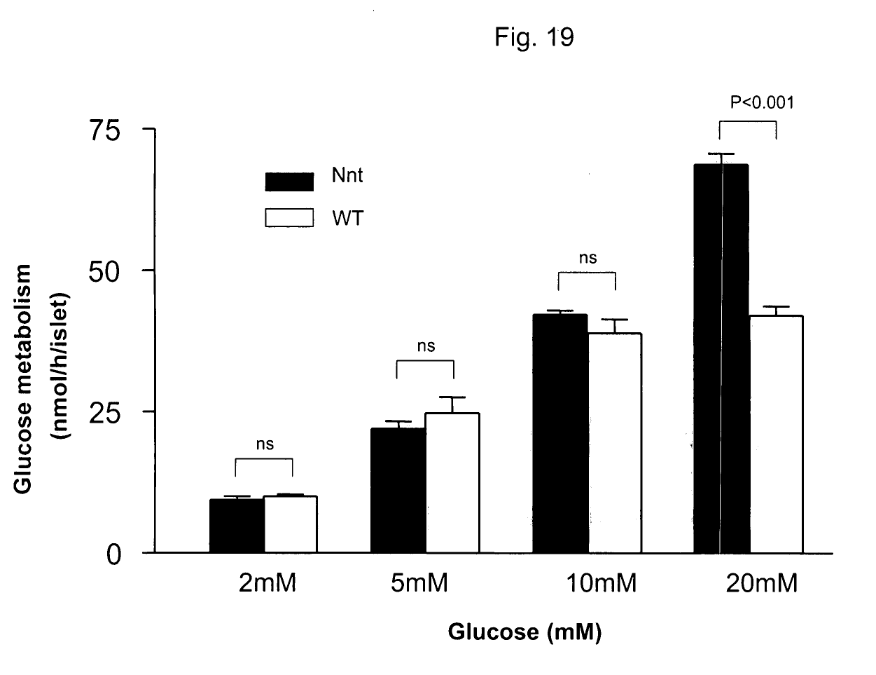

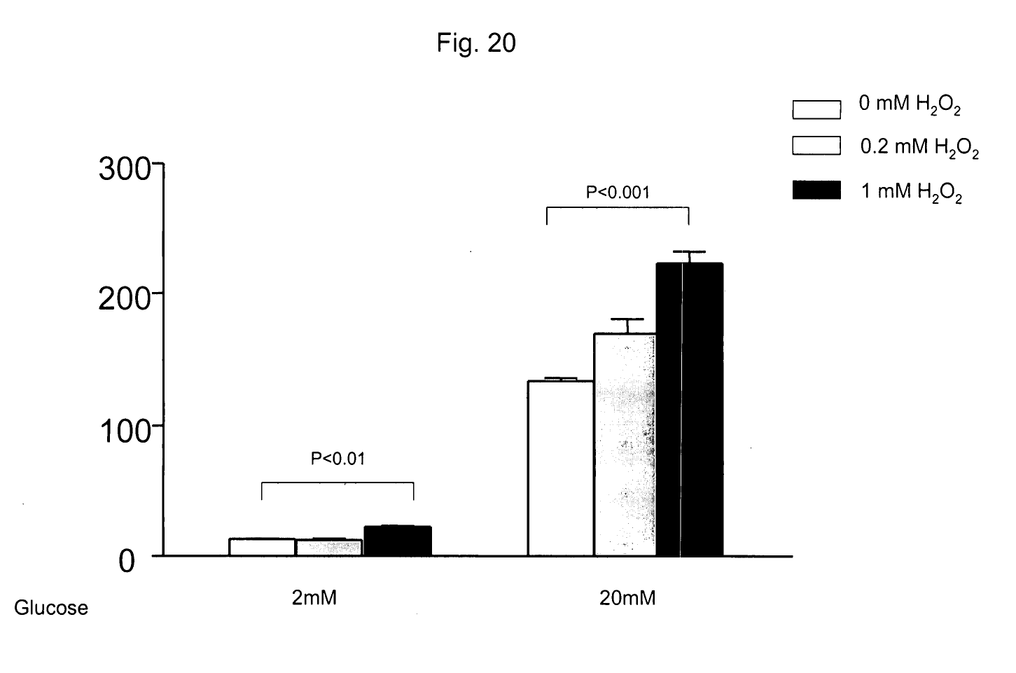

FIG. 19 shows that ATP production is reduced in pancreatic islets isolated from NNT mutant mice compared with wild-type mice, when glucose is increased to 20 mM. However, there is little difference at low glucose levels. If uncoupling protein is activated, one might expect that glucose utilization will be enhanced, since flux through the electron transport chain will increase although no ATP will be produced. To test this idea, glucose utilization was measured in islets isolated from NNT mutant mice and wild-type mice.

FIG. 19 shows that glucose utilization was similar at 2 mM glucose in pancreatic islets isolated from NNT mutant mice and wild-type mice. However, glucose utilization was significantly enhanced in NNT mutant mice at 20 mM glucose. This is consistent with an increase in mitochondrial uncoupling in NNT islets at high glucose levels.

One ROS that is expected to increase is H₂O₂. It is known that H₂O₂ inhibits insulin secretion, that it depolarizes the mitochondrial membrane potential and that it reduces ATP production. The depolarization of the mitochondrial membrane potential suggests that H₂O₂ may uncouple the mitochondria, perhaps by activating UCP2. If this were the case, we would expect that H₂O₂ should increase glucose utilization in islets isolated from NNT mutant mice and wild-type mice.

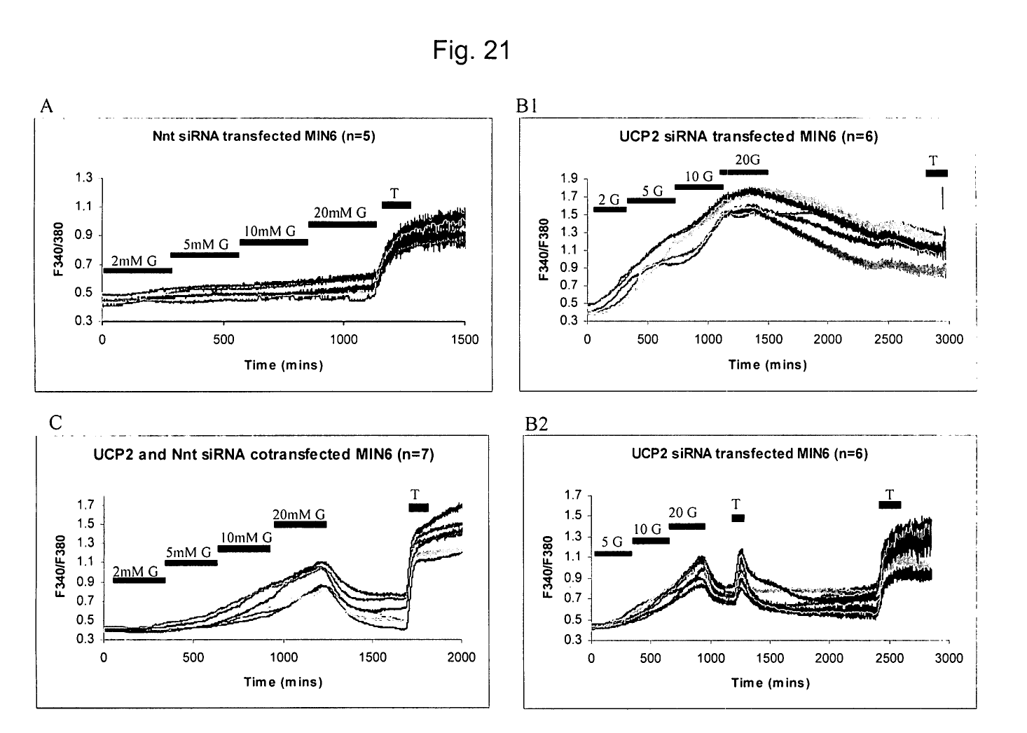

FIG. 21 shows that H₂O₂ significantly increases glucose utilization at 20 mM glucose in islets isolated from wild-type mice.

In the present Example the hypothesis is that enhanced ROS in NNT mutant mice inhibits ATP production by stimulating UCP2, and thereby prevents glucose inducing KATP channel closure, Ca entry and insulin secretion. If this is the case, then these effects of NNT should not be apparent in beta-cells of UCP2-knockout mice. In this case, ROS levels will rise but as they will not activate UCP2, they will not affect the mitochondrial membrane potential or ATP production. Thus glucose should cause KATP channel closure, Ca²⁺ entry and insulin secretion in beta-cells that lack both NNT and UCP2.

FIG. 21 shows that, as predicted, siRNA knockdown of NNT plus UCP2 restores the ability of MIN6 cells to respond to glucose with a rise in intracellular calcium, which is abolished when only NNT is knocked down.

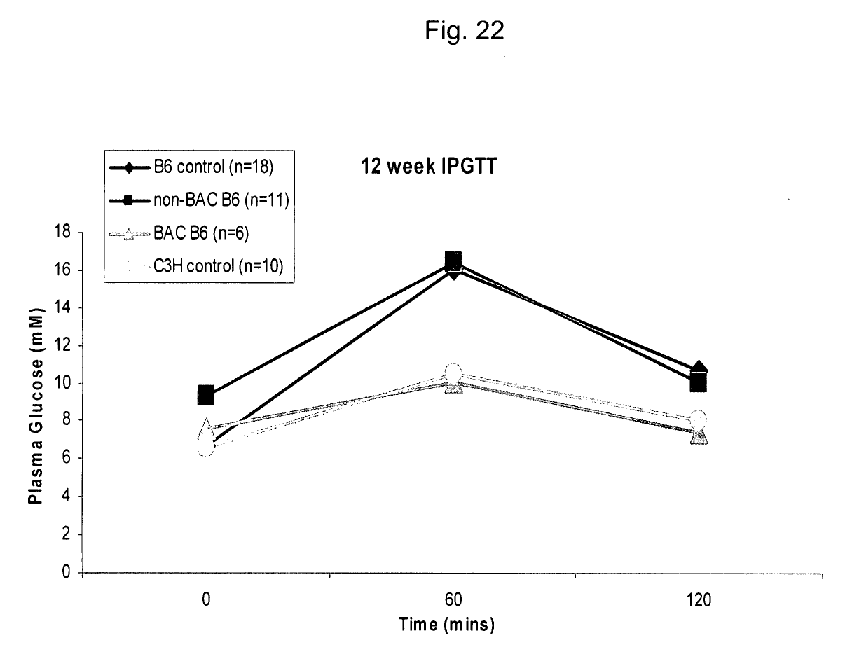

FIG. 22 shows that the glucose tolerance can be restored in C57BL/6J mice by a BAC transgene carrying the entire NNT gene. Without being bound to any theory or mechanism of action, it is postulated the BAC-encoded NNT may be complementing the deleted endogenous gene and thus it appears that impaired glucose tolerance in C57BL/6J mice is entirely the result of the mutation in NNT.

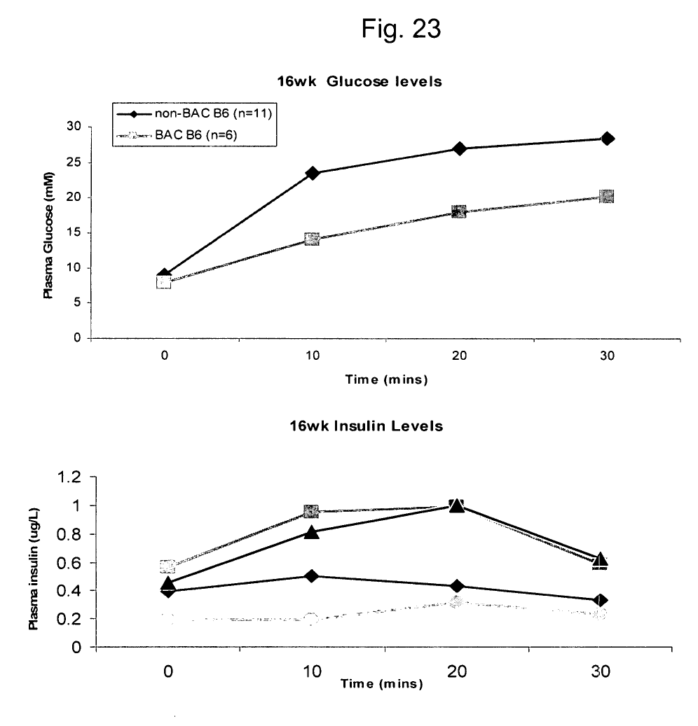

FIG. 23 shows in the same mice as in FIG. 22 that insulin secretion is restored in mice carrying the BAC and thus explains their improved glucose tolerance.

From the above data, it is demonstrated that down-regulation of Nnt results in the increase of free radicals in the cell via regulation of UCP2. This finding will have important implications in detoxification of free radicals/reactive oxygen species, not only in diabetes but also in other conditions caused by oxidative stress (e.g., motor neurone disease, ageing). It is known that free radical production and in particular ROS production is an indicator of altered mitochondrial function (see U.S. Pat. No. 6,140,067, incorporated herein by reference in its entirety). It is contemplated that the present findings may be used in methods of identifying an agent that modulates (and particularly enhances) the activity of wild-type NNT will be an agent that is useful in detoxification of free radical/ROSs in an animal. In such methods that agent may modulate (and especially enhance) the expression, activity or targeting of the NNT polypeptide to the mitochondria. The polypeptide in such methods is defined as described above for the identification of agents for the identification of agents that modulate glucose-stimulated insulin secretion. In like manner, either the polypeptide or the agent used in the identification assays of the present example can be linked to a solid support; the agent can be selected by identifying an agent that specifically binds to the polypeptide. The polypeptide may be an isolated protein or may be a polypeptide expressed in a recombinant cell or an intracellular organelle of such a cell. The cell may, but need not be, an insulin-secreting cell. Agents identified in such methods may be useful in the treatment of oxidative stress disorders.

### Example 5

### Further Studies to Corroborate Role of Nnt in Glucose Metabolism

Glucose Concentration Dependence of Insulin Secretion.

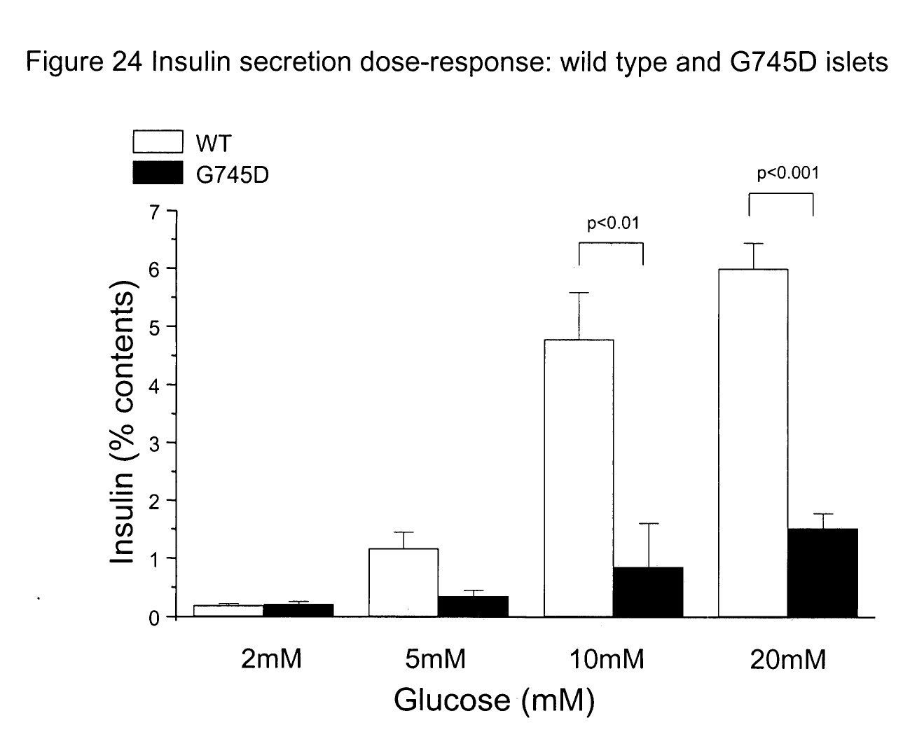

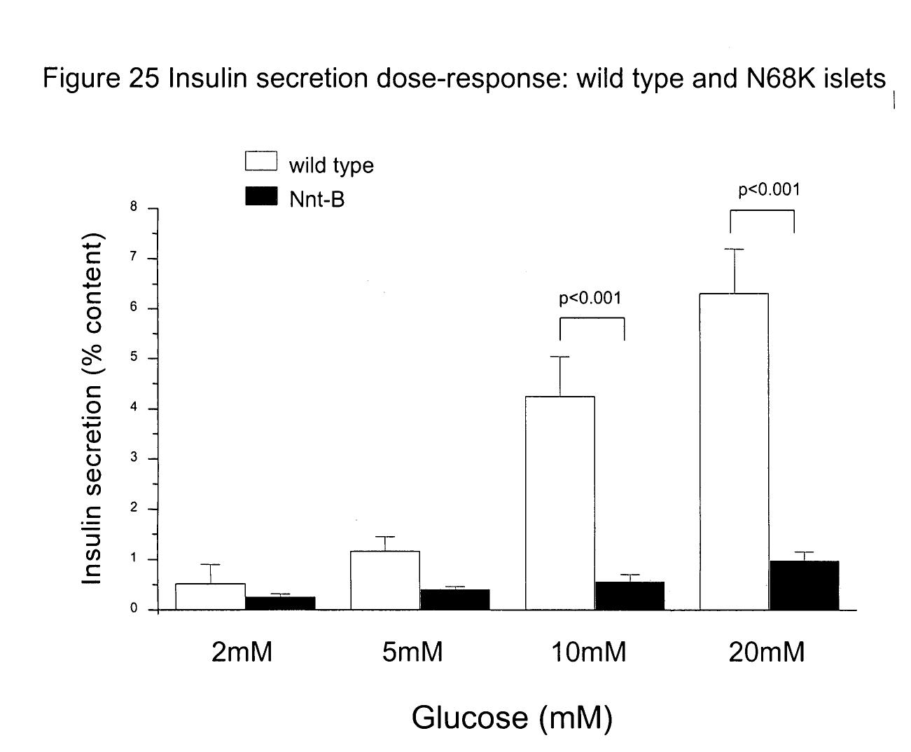

The studies discussed above demonstrate that insulin secretion in response to 10 mM glucose was abolished in pancreatic islets isolated from mice carrying mutations in Nnt (see also Freeman et al. Diabetes 55:2153-2156 (2006)). However, in IPGTT these mice secreted some insulin when blood glucose levels rose to around 20 mM. Therefore, insulin secretion from isolated islets was examined over a wider range of glucose concentrations. FIGS. 24 and 25 show that, whereas glucose concentrations above 5 mM stimulate secretion in wild-type islets, there is no significant release in response to either 5 or 10 mM glucose in islets isolated from N68K and G745D mice. At 20 mM glucose, however, there was a small but significant increase in insulin secretion from both N68K and G745D islets. These results explain the insulin secretion observed in vivo, and also explain why the mice do not suffer from overt diabetes, but only from impaired glucose tolerance.

Restoration of Glucose Tolerance and Glucose stimulated Insulin Secretion in C57BL/6J Mice Complemented for their Lack of Nnt.

Transgenic expression of the entire Nnt gene in C57BL/6J mice rescues the glucose-intolerant and impaired insulin secretion phenotype of these mice. This finding proves that Nnt is the gene underling inappropriate glucose homeostasis in C57BL/6J mice (see Freeman et al. Diabetes 55:2153-2156 (2006)).

Importance of the Mitochondrial Membrane Potential.

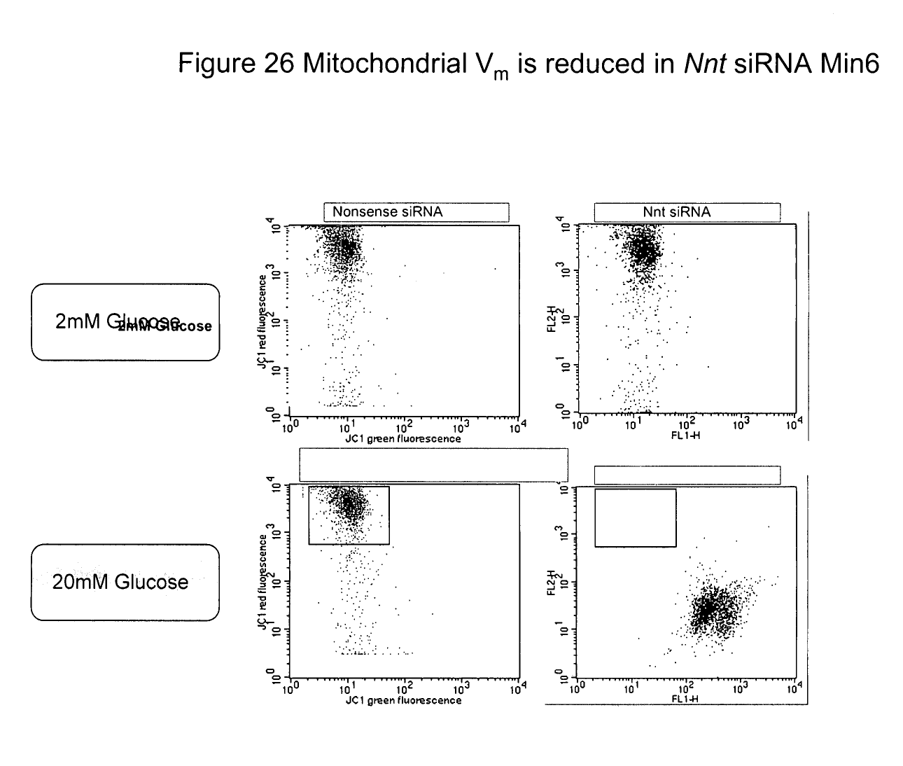

In order to test whether Nnt impairs ATP synthesis in beta-cells by reducing the mitochondrial membrane potential (mitoVm), the effects of glucose on mitoVm in MIN6 cells transfected with nonsense or Nnt siRNA was examined using the fluorescent dye JC-1. This dye fluoresces green when the membrane potential is low, but red when the membrane potential is increased. The ratio of red to green fluorescence is dependent on mitoVm alone and not on the size, shape or density of mitochondria within the cells. FIG. 26 shows that the average mitoVm was similar at 2 mM glucose, but that when glucose was increased to 20 mM, the mitochondrial membrane potential depolarized in MIN6 cells transfected with Nnt siRNA, but not with nonsense siRNA.

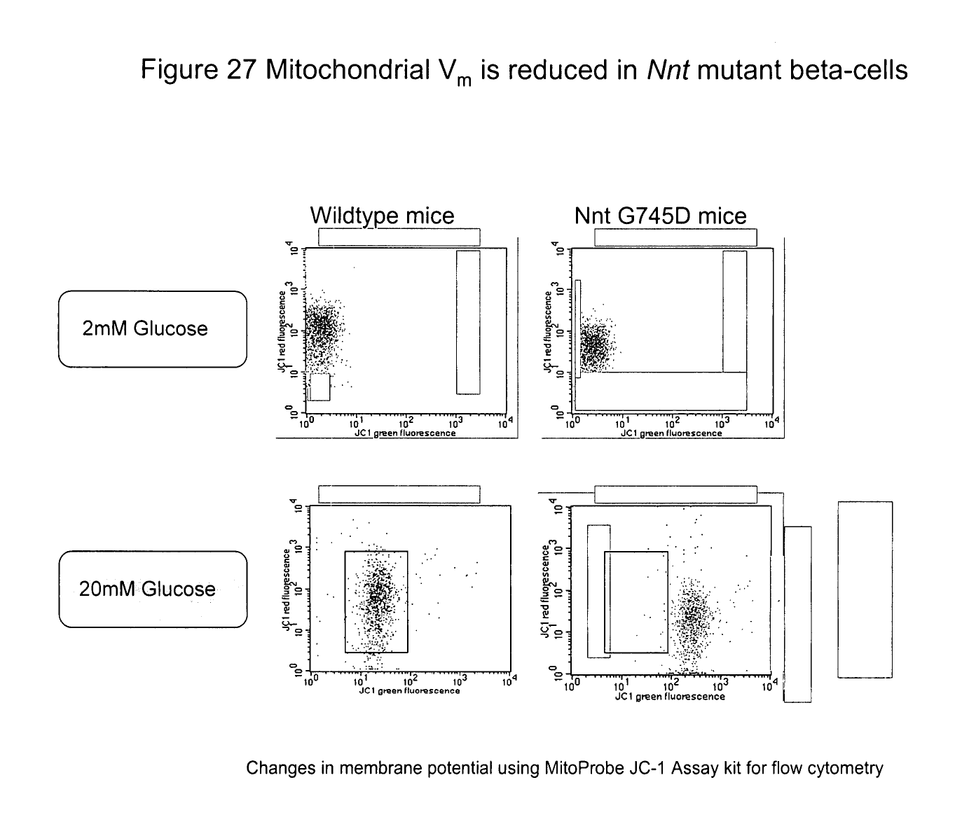

Similarly, mitoVm was the same in beta cells isolated from wild-type or Nnt-G745D mouse islets at 2 mM glucose (FIG. 27). When glucose was increased, there was a small depolarization of mitoVm in WT beta-cells but this was substantially larger in Nnt-G745D beta-cells.

In summary the above studies provide clear evidence of a change in mitochondrial membrane potential as a result of reducing levels of Nnt or mutating Nnt. This is consistent with the proposed model.

### Example 6

### Further Studies on Implications for ROS and Nnt in Insulin Secretion

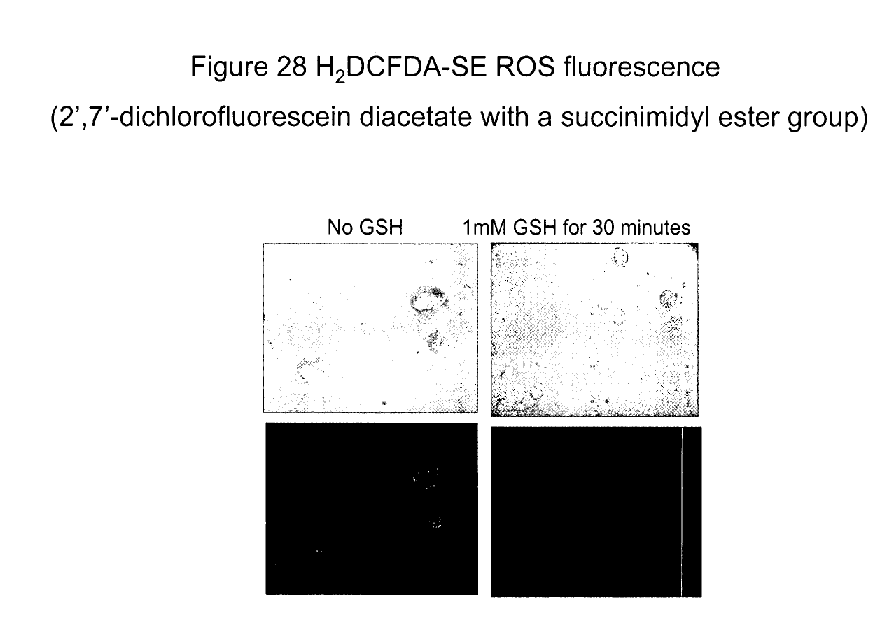

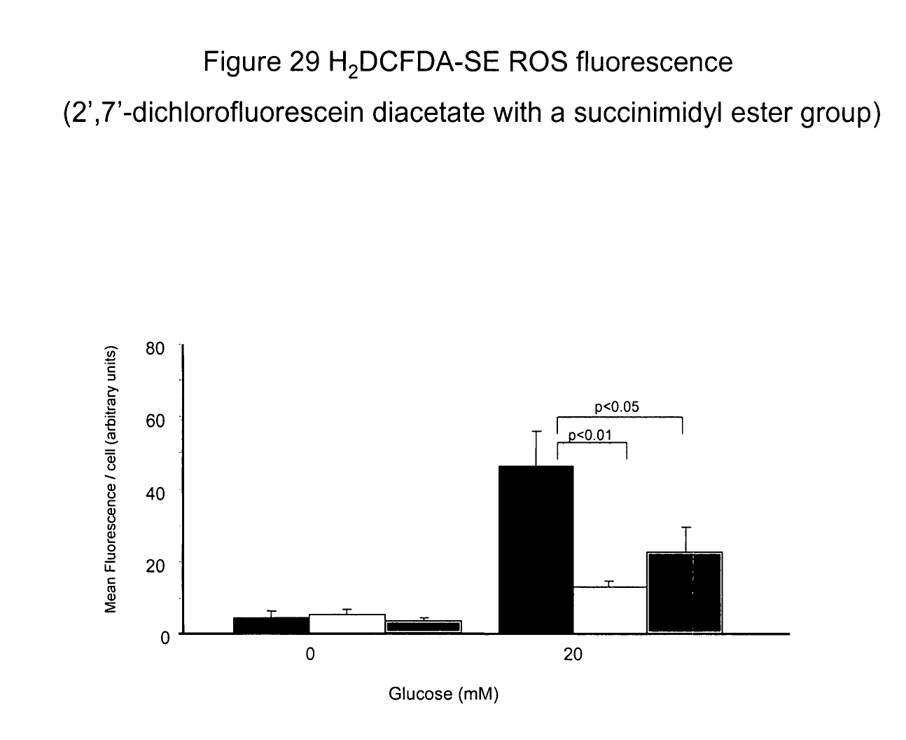

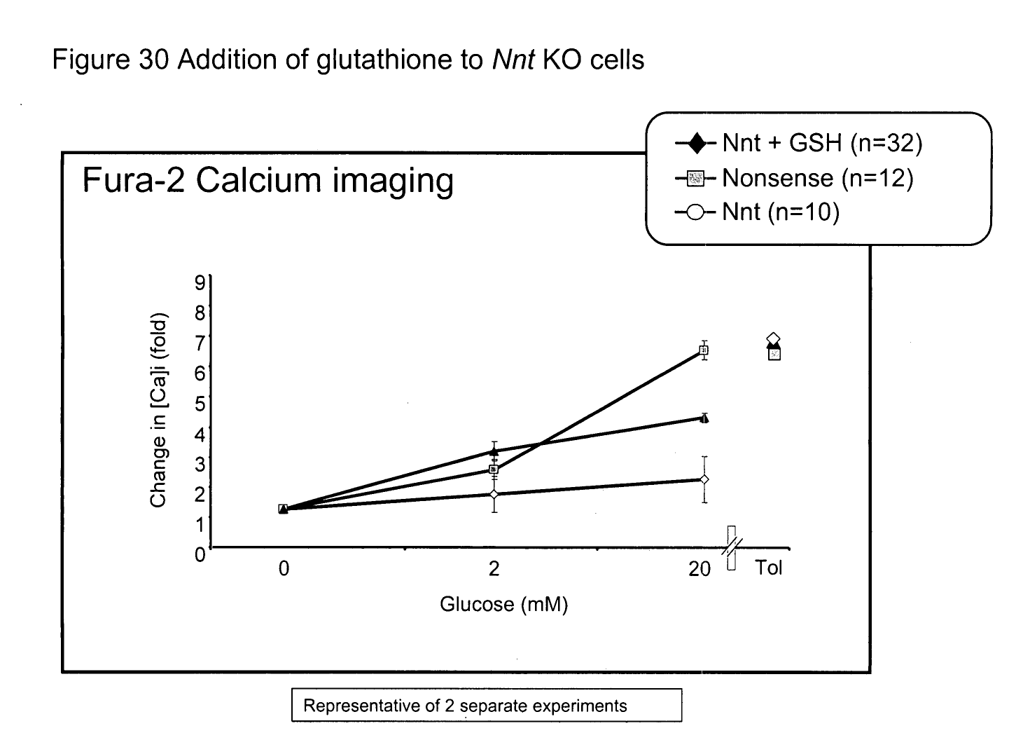

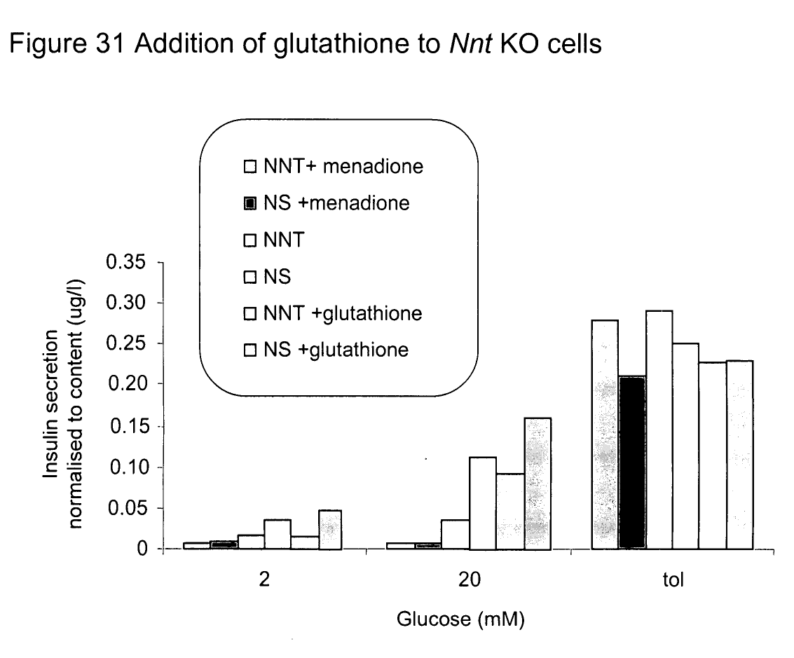

If, as suggested above, ROS generation underlies the impaired insulin secretion of Nnt pancreatic islets, then removal of excess ROS should restore secretion. Studies were carried out order to reverse oxidative stress and rescue insulin secretion using the antioxidant glutathione. FIGS. 28 and 29 show that incubation with 1 mM glutathione for 30 min decrease ROS production in MIN6 cells transfected with Nnt siRNA. Furthermore, incubation with 1 mM glutathione for 4 hours partially reversed the impaired calcium and insulin secretory responses found in MIN6 cells transfected with Nnt siRNA (FIGS. 30 and 31, respectively).

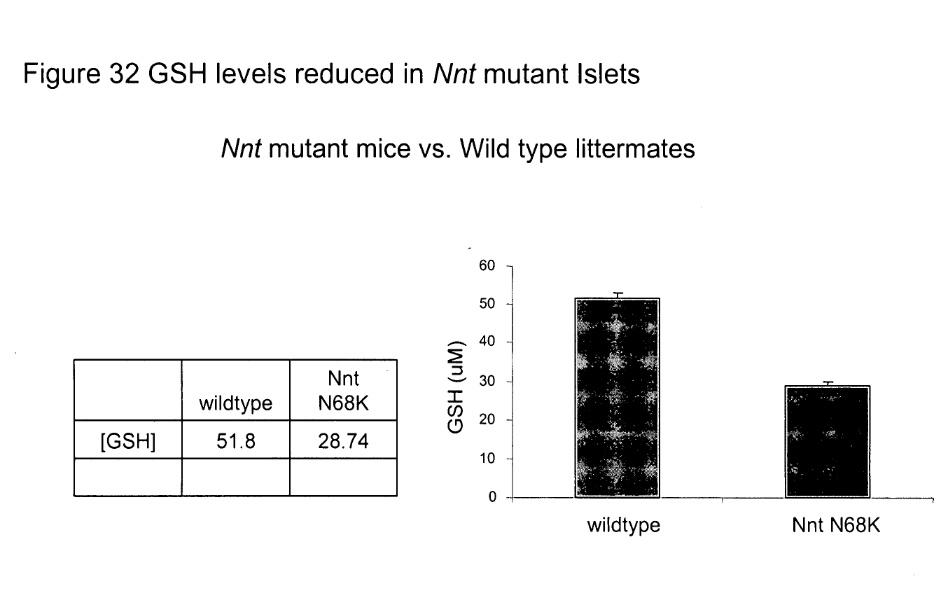

Consistent with a role for Nnt in production of reduced glutathione it was shown that reduced glutathione levels are lower in Nnt mutant pancreatic islets (FIG. 32).

These data support the hypothesis that ROS is part of the mechanism ultimately controlling insulin secretion and that Nnt mutants exert their effects through changes in ROS levels.

### Example 7

### Further studies on Role of Nnt in UCP2 Stimulation

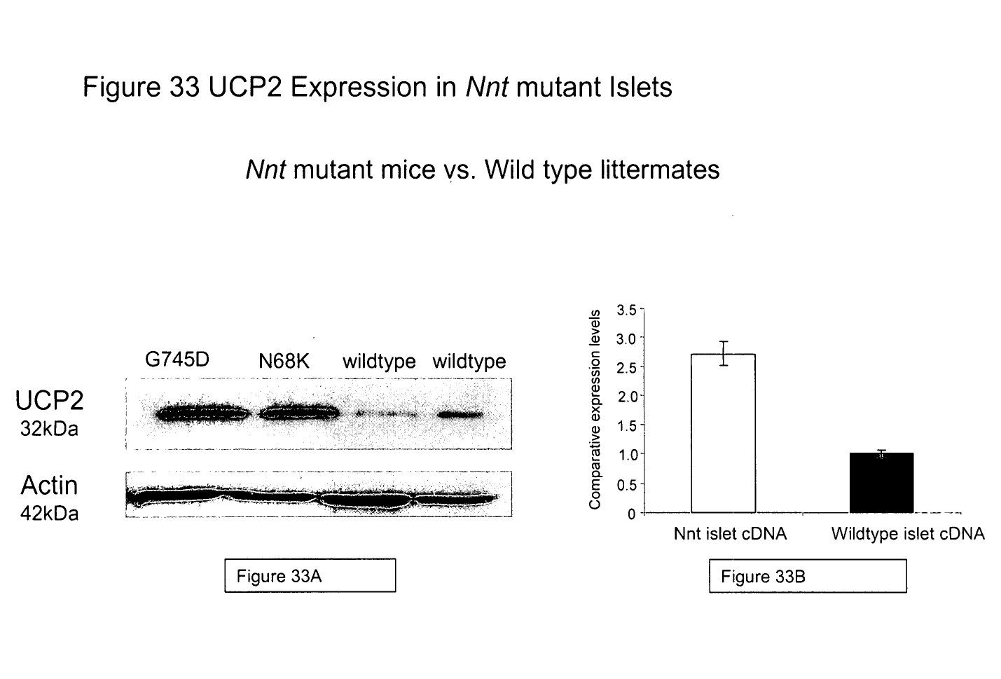

In the studies described above, UCP2 was shown to be upregulated in Nnt pancreatic islets (see also FIG. 33), perhaps as a consequence of ROS elevation. If this prediction is correct, deletion of UCP2 should restore glucose-dependent ATP synthesis, K_{ATP} channel closure, Ca²⁺ elevation, and insulin secretion in beta-cells lacking Nnt. Therefore, further studies were performed using siRNA to knock down Nnt and UCP2 simultaneously, or singly, in MIN6 cells.

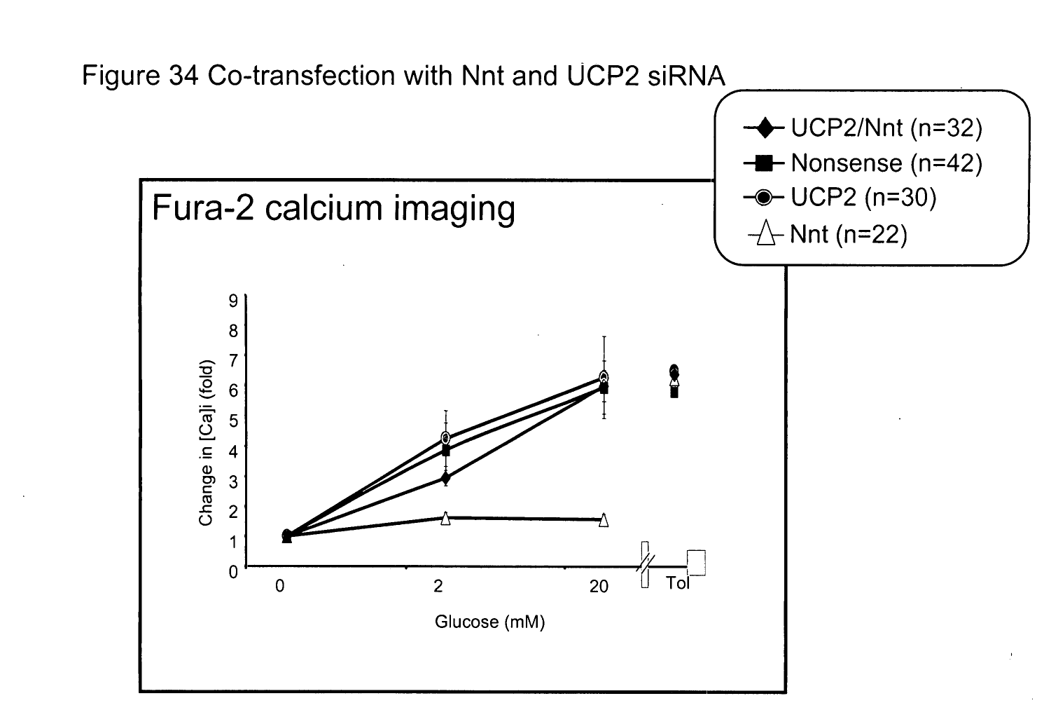

FIG. 34 shows that the calcium response to glucose was unaffected by knockdown of UCP2 alone. Knockdown of Nnt alone almost completely abolished the calcium response, as previously reported. However, the calcium response could be largely restored by simultaneous knockdown of UCP2 and Nnt.

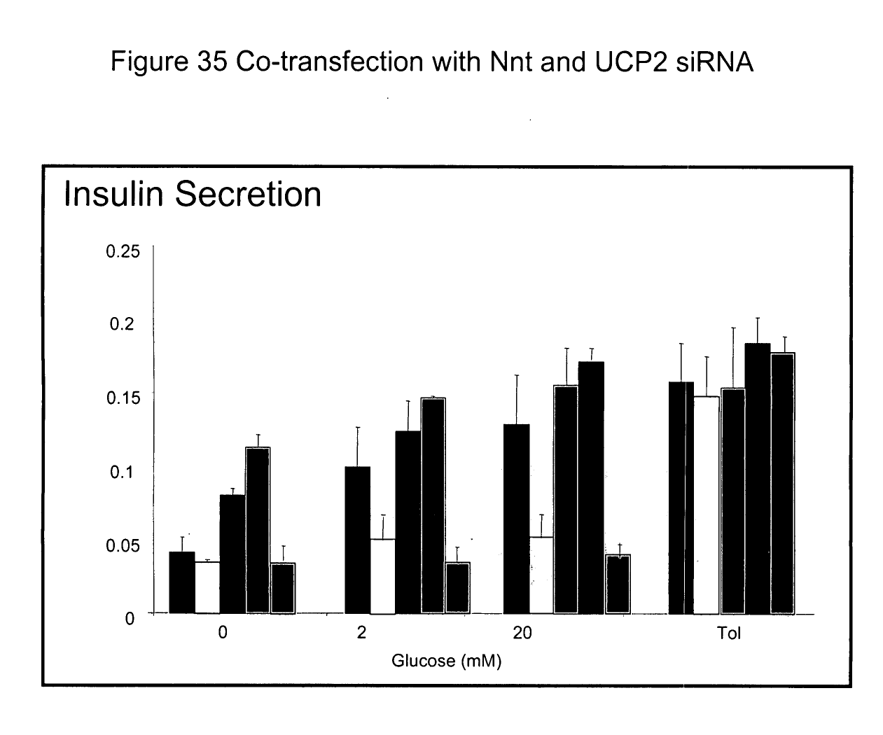

FIG. 35 shows that equivalent results were obtained for insulin secretion. Thus simultaneous knockdown of UCP2 and Nnt reversed the impaired secretory response to glucose observed for Nnt knockdown alone. Insulin secretion at basal (0 mM) glucose was also was also enhanced in cells transfected with either UCP2 or UCP2+Nnt siRNAs.

Tolbutamide, which bypasses cell metabolism and closes K_{ATP} channels directly, stimulated a calcium response and insulin secretion under all conditions (FIGS. 34 and 35). These results are consistent with the idea that Nnt acts by stimulating UCP2 function, either via enhanced expression of UCP2, or by increasing UCP2 activity directly, or both.

Further studies are being carried out to corroborate this finding in vivo. In such studies, UCP-2 knockout mice are crossed with the three Nnt mutant mouse lines described above. The mice generated from these crosses will be monitored in order to measure glucose tolerance, insulin secretion in vivo and insulin secretion in isolated pancreatic islets. ROS levels also will be determined. The examples presented herein above provide a teaching of how to monitor characteristics such as ROS levels, glucose tolerance, insulin secretion in vivo and insulin secretion in isolated islets. Routine techniques can be used to monitor these parameters which will readily lead to a corroboration of whether Nnt acts to stimulate UCP2 in vivo.

### Example 8

### Effects of Metformin

Metformin is widely used to treat type-2 diabetes. It is thought to act by stimulating (indirectly) the activity of AMPK. However, the molecular target of the drug is unknown. One idea is that it acts by reducing mitochondrial metabolism and elevating AMP levels, although this has been contested. Another is that it reduces oxidative stress. Preincubation in 15 μM metformin, a concentration similar to that found in the plasma of patients treated with this drug, protects human and rodent pancreatic islets from the deleterious effects of elevated lipids, and partially restores insulin secretion in type-2 diabetic human islets. Incubation of rodent islets at higher concentrations (1 mM) reduces glucose-stimulated insulin secretion, suggesting metformin has multiple actions.

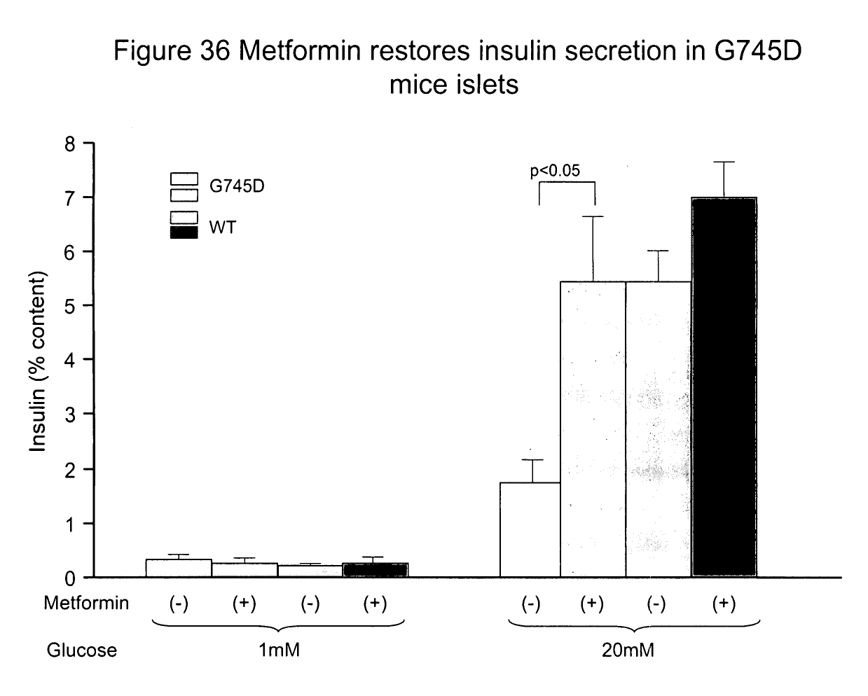

In preliminary studies, it was discovered that a 24-hour preincubation in 15 μM metformin surprisingly restored glucose-stimulated insulin secretion in Nnt-G745D islets, while having little effect on WT islets (FIG. 36). Acute exposure to metformin was without effect. One possibility is that metformin acts by decreasing Complex I activity and ROS production. If so, its effect on ATP synthesis, as opposed to ROS production, must be small, since insulin secretion requires ATP. Alternatively, metformin may act at another stage of the secretory pathway.

### Example 9

### Dye-Based High Throughput Screening Assay to Screen for Compounds that Affect NNT Activity

In exemplary embodiments, a high throughput assay to screen for compounds that affect NNT activity may be based on the method described by Farrelly et al. Analytical Biochemistry 293:269-276 (2001). Exemplary such HTS assays are described in Example 10 herein below.

NNT couples proton translocation across the inner mitochondrial membrane with generation of NADPH. This protein may therefore act in ways similar to an uncoupler and thus may affect mitochondrial membrane potential, intramitochondrial pH and intramitochondrial calcium concentrations. Compounds that activate or inhibit NNT would lead to changes in intramitochondrial calcium. This would allow an HTS screen based on mitochondrial calcium levels. Experiments could readily be carried out in insulin-secreting cells lines (e.g. MIN6) or even isolated beta-cells (methods to prepare these are already described herein above). In some aspects, a negative control can be used. A good negative control would be to repeat the experiment in cells is which Nnt is either down-regulated (e.g. by siRNA as described herein above) or lacking due to genetic deletion (the C57BL/6J mice could be used—these mice have a deletion mutation that results in a complete lack of detectable protein).

In an exemplary screening assay, MIN6 cells are cultured in for example microtitre plates. The intramitochondrial calcium in these cultured cells is measured using fluorescent dyes such as Rhod2 (Molecular probes Invitrogen Technologies, The Handbook, A guide to Fluorescent Probes and Labelling Technologies, Tenth Edition, 2005, by R. P. Haugland EDITOR M. T. Z. Spence ISBN 0-9710636-4-8; Boitier et al. 1999 J. Cell Biol. 145:795-868; Mitochondria exert a negative feedback on the propagation of intracellular Ca+ waves in rat cortical astrocytes). If ROS are present they will cause depolarization of mitochondrial membrane potential which is expected to elevate intramitochondrial calcium. The activity of Nnt will regulate the amount of intramitochondrial ROS and thus, indirectly, intramitochondrial calcium.

The experimental conditions can be varied. For example, it will probably be useful to elevate ROS either using 20 mM glucose or low concentrations of ROS generators such as menadione, hydrogen peroxide. This will enable one to more easily identify compounds that inhibit and activate Nnt.

The screening assay developed and tested in the microtitre plate format can then readily be adapted for high throughput screening in microtitre plate format using liquid handling robots to facilitate high throughput screening essentially as describe in Farrelly et al. Analytical Biochemistry 293:269-276 (2001).

### Example 10

### Yeast-Based High Throughput Screening Assay to Screen for Compounds that Affect NNT Activity

A yeast-based assay for high throughput screening can be used to identify compounds that will affect NNT activity. Yeast lack NNT and various uncoupling proteins. NNT couples proton translocation across the inner mitochondrial membrane with generation of NADPH. This protein may therefore act in ways similar to an uncoupler and thus may affect mitochondrial membrane potential, intramitochondrial pH and intramitochondrial calcium concentrations. Thus, the method of Farrelly et al. (Analytical Biochemistry 293:269-276 (2001)) could be modified to achieve expression of NNT (instead of UCP-1 as described in Farrelly et al.) in yeast which would allow an HTS screen based on mitochondrial membrane potential. Compounds that activate or inhibit NNT would lead to changes in mitochondrial membrane potential.

Expression of human NNT in yeast (Saccharomyces cerevisiae, S. pombe or any other suitable yeast species) may be achieved through conventional methods, for example by cloning in the pYES2 vector (Invitrogen Life Technologies) or other suitable vector. The yeast cells are then permeabilized using any conventional method, for example by the high potassium acetate/freeze method (Farrelly et al.) or any other suitable permeabilizing method.

The membrane potential of the permeabilized yeast is measured using fluorescent dyes such as DiSC3 (Farrelly et al.) or JC-1 (Molecular probes Invitrogen Technologies, The Handbook, A guide to Fluorescent Probes and Labelling Technologies, Tenth Edition, 2005, by R. P. Haugland EDITOR M. T. Z. Spence ISBN 0-9710636-4-8, see also, See also Smiley et al., PNAS 88: 3671-3675 (1991); Reers et al. Biochemistry 30 (18) 4480-4486 (1991); Cossarizza et al., Biochem Biophys Res Commun. November 30; 197(1):40-5 (1993). Kits for measuring membrane potential using JC-1 are commercially available through a variety of sources including e.g., Invitrogen, Biotium and Stratagene.

The HTS screen is carried out a in microtitre plate format using liquid handling robots to facilitate high throughput essentially as described in Farrelly et al. Analytical Biochemistry 293:269-276 (2001).

As an alternative or in addition to measuring membrane potential, the HTS also may be set up to measure measure mitochondrial pH with dyes such as SNARF-1 AM ester acetate after loading and selective retention (allowing for example 4 hours for the dye to wash out of the cytoplasmic compartment) in the mitochondria (Molecular probes Invitrogen Technologies, The Handbook, A guide to Fluorescent Probes and Labelling Technologies, Tenth Edition, 2005, by R. P. Haugland EDITOR M. T. Z. Spence ISBN 0-9710636-4-8; See also Takahashi et al. BioTechniques Vol. 30, No. 4: pp 804-815 (2001)).

In still another alternative or in addition to measuring membrane potential, and/or mitochondrial pH, a high throughput screen also can be set up to measure changes in intramitochondrial calcium concentrations by expressing a mitochondrial targeted protein such as Aequorin. This can be achieved by adding the appropriate mitochondrial sorting peptide to the expression fusion protein construct as describe for example by Rizzuto et al. (Nature 358, 325-327 (1992)), Rutter et al. (PNAS 93: 5489-5494 (1996) and Kennedy et al. (J. Clin. Invest. 98:2524-2538 (1996)). Alternatively the dye Rhod2 or its derivatives could be used to measure mitochondrial calcium. As above, the HTS screen can be readily set up and automated as described by Farrelly et al. Analytical Biochemistry 293:269-276 (2001).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. In certain of the above examples, articles are identified using a number in square brackets. The following is a listing of the references that correspond to those number.

We claim:

1. A method of identifying an agent that modulates glucose-stimulated insulin secretion in an animal, the method comprising the steps of: (i) contacting an agent to a wild-type NNT polypeptide comprising at least 20 contiguous amino acids of SEQ ID NO:2 or SEQ ID NO:4; (ii) selecting an agent that binds to the polypeptide or modulates the expression or activity of the polypeptide, or its targeting to the mitochondria and (iii) determining the effect of the selected agent on glucose-stimulated insulin secretion, thereby identifying an agent that modulates glucose-stimulated insulin secretion in an animal.

2. A method of identifying an agent that modulates glucose-stimulated insulin secretion in an animal or cell, the method comprising the steps of: (i) contacting an agent with an NNT polypeptide; and (ii) selecting an agent that modulates the expression or activity of the polypeptide or its targeting to the mitochondria, thereby identifying an agent that modulates glucose-stimulated insulin secretion in an animal.

3. The method of claim 1 or 2, wherein step (ii) comprises selecting an agent that modulates the expression of the polypeptide.

4. The method of claim 1 or 2, wherein step (ii) comprises selecting an agent that modulates the activity of the polypeptide.

5. The method of claim 1 or 2, wherein step (ii) comprises selecting an agent that modulates the correct targeting of the polypeptide to the mitochondria.

6. The method of claim 1 or 2, wherein the polypeptide comprises SEQ ID NO: 2 or SEQ ID NO:4.

7. The method of claim 1 or 2, wherein the polypeptide is encoded by a nucleic acid sequence of SEQ ID NO: 1 or SEQ ID NO:3.

8. The method of claim 1 or 2, wherein the polypeptide is linked to a solid support.

9. The method of claim 1 or 2, wherein the agent is linked to a solid support.

10. The method of claim 1 or 2, wherein the agent is selected by identifying an agent that specifically binds to the polypeptide.

11. The method of claim 1 or 2, wherein the mixture comprises a cell expressing the polypeptide.

12. The method of claim 1 or 2, wherein the activity of the polypeptide is determined by a step comprising measuring a change in calcium flux in said cell.

13. The method of claim 12, wherein the activity of the polypeptide is determined by a step comprising measuring a change in membrane potential of a cell.

14. The method of claim 12, wherein the cell is an insulin-secreting cell or an intracellular organelle of such a cell.

15. The method of claim 12, wherein the activity of the polypeptide is determined by a step comprising measuring a change in insulin secretion by the cell.

16. The method of claim 1 or 2, wherein the activity of the polypeptide is determined by the step comprising measuring a change in glucose-stimulated insulin secretion by the cell.

17. The method of claim 1 or 2, the method further comprising administering the agent to a diabetic animal and testing the animal for increased glucose-stimulated insulin secretion.

18. The method of claim 17, wherein said animal has a mutant NNT activity that causes an impaired β-cell function and said agent improves said impaired β-cell function.

19. A method of inducing glucose-stimulated insulin production in an animal, the method comprising administering a therapeutically effective amount of the agent selected in claim 1.

20. The method of claim 19, wherein the animal is a human.

21. The method of claim 19, further comprising administering metformin to said animal.

22. A method of determining whether a patient is susceptible to developing polygenic type 2 diabetes comprising determining the presence of a mutation in the NNT gene of said patient, wherein a mutation in the NNT gene of said patient is indicative of a susceptibility to polygenic type 2 diabetes.

23. A recombinant cell transfected with the polynucleotide that encodes an NNT polypeptide wherein said cell is an insulin secreting cell.

24. The cell of claim 23, wherein said cell expresses a mutant NNT protein.

25. The cell of claim 23, wherein said cell expresses a wildtype NNT protein.

26. The cell of claim 23, wherein the cell is a pancreatic islet cell.

27. A method of treating diabetes in a patient, comprising: administering to said patient an effective amount of an agent that augments, potentiates or otherwise increases the activity of nicotinamide nucleotide transhydrolase (NNT) in said patient.

28. The method of claim 27, wherein said patient has a mutant NNT and said agent is administered to overcome the effects of said mutation.

29. The method of claim 27, wherein said effects of said mutation comprise a reduced insulin secretion, a glucose-dependent increase in [Ca²⁺]i in the β-cells of said patient, an increased glucose intolerance, impaired glucose dependent β-cell electrical activity, a decrease in β-cell ATP production, or enhanced K_{ATP} channel activity.

30. The method of claim 27, wherein said method comprises administering a composition that comprises a wild-type NNT to said patient.

31. The method of claim 27, wherein said agent enhances NNT expression.

32. The method of claim 27, wherein said agent enhances NNT activity.

33. The method of claim 27, wherein said patient has Type 2 Diabetes.

34. The method of claim 27, wherein said patient has impaired the β-cell function and said administration improves said β-cell function.

35. The method of claim 27, wherein said administering results in increased islet blood flow, increased pancreatic β-cell perfusion, reduced insulin resistance in skeletal muscles, increased insulin-mediated glucose disposal, or increased insulin-mediated glucose uptake by skeletal muscles.

36. The method of claim 33, further comprising administering to said patient a therapeutically effective amount of a sulfonylurea agent or a biguanide agent.

37. The method of claim 36 wherein said biguanide is selected from the group consisting of metformin, phenformin and buformin.

38. The method of claim 33, further comprising administering to said patient a therapeutically effective amount of metformin.

39. A method of screening for an agent that modulates NNT activity comprising culturing a cell according to claim 23 and determining the mitochondrial calcium levels in said cell being cultured in prior to and after addition of a candidate agent that modulates NNT activity wherein an alteration in the mitochondrial calcium level of said cell in the presence of said candidate agent identifies said agent as an NNT modulator.

40. The method of claim 39, further comprising elevating the reactive oxygen species (ROS) of said cell in culture prior to addition of said candidate agent.

41. The method of claim 40, wherein said ROS is elevated by addition of glucose, menadione, hydrogen peroxide or a combination thereof.

42. A method of screening for an agent that modulates NNT activity comprising culturing a yeast cell transformed or transfected with the polynucleotide that encodes an NNT polypeptide and determining the membrane potential and/or mitochondrial pH in said yeast cell being cultured prior to and after addition of a candidate agent that modulates NNT activity wherein an alteration in the membrane potential and/or mitochondrial pH of said cell in the presence of said candidate agent identifies said agent as an NNT modulator.


SEQUENCE LISTING (16 sequences)

SEQ ID NO: 1 - DNA, 4600 bp, Homo sapiens
ggatttttcctagcgagctggaggaaaggcaggtacccggagtggagttatattacctgtttctttgttctgatgggaccccagagaggacatcacgctttgtaactggttcttcaaggggagaagcaaatgtgtccctgaaaacttcagctgccgcgtttggcaccagtgatttgccttcaaggaaactggggagtcagaaaattgggaactcatatcaacatggcaaacctattgaaaacagtggtgactggctgctcgtgtcctctacttagcaatttggggtcctgtaagggtctacgtgtgaagaaggattttttacgaacattttatactcaccaagaactgtggtgtaaagcgcctgtaaaaccaggaattccatataagcaactgactgttggagtccccaaagagatattccaaaatgagaagcgagtggcattgtctcctgctggtgttcagaacttggtcaagcagggttttaatgttgtcgtggaatcgggtgcgggcgaagcttccaagttctcagatgatcactatagagtggcaggtgcccaaatccaaggggcaaaggaagtgctggcttctgatttggtggtcaaagtgcgagcccctatggttaatccaacattaggtgttcatgaagctgaccttttaaagacatcaggaacgctgattagttttatttacccagcccaaaatccagagttgctaaataaactttcccaaagaaaaactacagttctggcaatggaccaggttccaagagtcacaattgctcagggatatgatgcgctaagctccatggccaacattgcgggttataaggctgttgtcctagcagcaaatcattttgaacgtttttttactggtcagatcacagctgctggaaaagttcctccagctaagattctgatagttggtggtggtgttgctgggcttgcttctgcaggcgcagcaaagtcgatgggtgcaattgttcgaggatttgacacaagagctgcagctttggaacagttcaagtctcttggtgctgagcccttggaggtggacttgaaggaatctggtgagggacaaggaggatatgcaaaagagatgtccaaagagttcattgaagctgaaatgaaactctttgctcaacaatgcaaggaggtagacatccttatcagcacagcacttattccaggtaaaaaagctccagttttatttaataaagaaatgattgagtcaatgaaggaaggttcagttgttgtggatttagctgctgaggctggtggaaactttgaaaccactaagccaggagaactctacattcataagggaattactcacataggctacacagacctgcccagccgaatggccactcaggccagcaccctatattccaacaacatcaccaaactcctgaaggccatcagcccggacaaagataatttttattttgatgtgaaagatgactttgactttggtacgatgggtcatgtcattagaggaactgtagtgatgaaagatggtaaagtgattttcccagctcccacaccgaaaaatattcctcaaggtgccccagtaaaacagaagacagtggctgagctggaagctgaaaaagcagctaccattacacccttcaggaagacaatgtcaacggcttctgcatatacagcaggtctcacagggatactgggtttgggcattgcggctcccaatctagccttttctcagatggtgaccacttttggcttggctggcattgtggggtatcataccgtctggggagtgacccctgctctccactcaccactgatgtctgtgacaaatgcaatctcagggctgactgcagttggtgggttggcactgatgggaggacatttgtatccttccacaacttctcagggccttgctgctcttgctgcattcatatcctctgtcaacattgcaggtggctttctggtgactcagagaatgctggacatgttcaagcgtcccactgaccccccagaatacaactacctgtacctgctccctgccggcacctttgttggtggatatttagctgccctctacagtggttataacattgaacagatcatgtacctaggctcgggtttgtgctgtgtcggtgccttggctggcctctccacccagggaacagcacgtcttggcaatgcactgggcatgattggggttgctggaggactggcagccaccctcggagtcctaaaaccgggcccagaattactagctcagatgtctggagcgatggctttgggtggtaccattggattgacaattgccaaacgcatccagatttctgatttacctcaattagttgctgcttttcacagtttagtgggtttggcagctgtacttacttgcatagctgagtacattatagaatatccacattttgctacggacgcagcagcaaatctcaccaagattgtggcctacctcggcacttacattggtggcgtcacctttagtgggtctctcattgcctatggaaaattgcagggtctcctgaaatctgcccctctcctactgcctggaaggcacttactcaatgcaggcttactggctgctagtgtgggcgggataatcccattcatggtggacccaagctttactactggcatcacctgtctgggttcagtgtctgctctctctgctgtcatgggtgtgactttgacagctgctattgggggtgctgacatgcccgtcgttatcactgtgctgaacagctactcaggctgggccctgtgtgcagagggcttcctgctcaacaacaatctgctgaccatcgtgggtgcactcataggctcgtctggtgctatcctgtcatacatcatgtgtgtggcaatgaatcgctccctggctaatgtgattcttggaggctatggcaccacttcaacagctggtggaaaacccatggaaatttctggcacacatacggaaatcaaccttgacaatgcaattgacatgattcgagaagctaatagcattattattacaccaggctatggtctctgtgcagccaaagctcaataccccattgctgatttggtaaagatgctcactgagcaaggcaaaaaagtcaggtttggaattcacccagttgcaggccgaatgcctggtcagcttaatgtgctgctggctgaggctggtgtgccatatgacattgtgttggaaatggatgagatcaaccatgattttccagatactgatttggtccttgtaattggagctaatgacactgttaattcagcagctcaagaagatcccaactctattattgcaggcatgccagtccttgaggtctggaaatcaaagcaggtgattgttatgaagaggtctttgggtgttggctatgctgcagtggacaatccaatcttctacagacctaacacggccatgcttctaggtgatgccaagaaaacatgtgacgcgctccaggcgaaagttagagaatcctatcagaagtaaatattaaggatcaagctgttagctaataatgccacctctgcagttttgggaacaggcaaataaagtatcagtatacatggtgatgtacatctgtagcaaagctcttggagaaaatgaagactgaagaaagcaaagcaaaaactgtatagagagatttttcaaaagcagtaatccctcaattttaaaaaaggattgaaaattctaaatgtctttctgtgcatattttttgtgttaggaatcaaaagtattttataaaaggagaaagaacagcctcattttagatgtagtcctgttggattttttatgcctcctcagtaaccagaaatgttttaaaaaactaagtgtttaggatttcaagacaacattatacatggctctgaaatatctgacacaatgtaaacattgcaggcacctgcattttatgtttttttttttcaacaaatgtgactaatttgaaacttttatgaacttctgagctgtccccttgcaattcaaccgcagtttgaattaatcatatcaaatcagttttaattttttaaattgtacttcagagtctatatttcaagggcacattttctcactactattttaatacattaaaggactaaataatctttcagagatgctggaaacaaatcatttgctttatatgtttcattagaataccaatgaaacatacaacttgaaaattagtaatagtatttttgaagatcccatttctaattggagatctctttaatttcgatcaacttataatgtgtagtactatattaagtgcacttgagtggaattcaacatttgactaataaaatgagttcatcatgttggcaagtgatgtggcaattatctctggtgacaaaagagtaaaatcaaatatttctgcccgttacaaatatcaaggaagacctgctactatgaaatagatgacattaatctgtcttcactgtttataatacggatggattttttttcaaatcagtgtgtgttttgaggtcttatgtaattgatgacatttgagagaaatggtggctttttttagctacctctttgttcatttaagcaccagtaaagatcatgtctttttaaaaaaaaaaaaaaaaaaaaaaaaaaaaaaaaa

SEQ ID NO: 2 - protein, 1086 aa, Homo sapiens
MANLLKTVVTGCSCPLLSNLGSCKGLRVKKDFLRTFYTHQELWCKAPVKPGIPYKQLTVGVPKEIFQNEKRVALSPAGVQNLVKQGFNVVVESGAGEASKFSDDHYRVAGAQIQGAKEVLASDLVVKVRAPMVNPTLGVHEADLLKTSGTLISFIYPAQNPELLNKLSQRKTTVLAMDQVPRVTIAQGYDALSSMANIAGYKAVVLAANHFERFFTGQITAAGKVPPAKILIVGGGVAGLASAGAAKSMGAIVRGFDTRAAALEQFKSLGAEPLEVDLKESGEGQGGYAKEMSKEFIEAEMKLFAQQCKEVDILISTALIPGKKAPVLFNKEMIESMKEGSVVVDLAAEAGGNFETTKPGELYIHKGITHIGYTDLPSRMATQASTLYSNNITKLLKAISPDKDNFYFDVKDDFDFGTMGHVIRGTVVMKDGKVIFPAPTPKNIPQGAPVKQKTVAELEAEKAATITPFRKTMSTASAYTAGLTGILGLGIAAPNLAFSQMVTTFGLAGIVGYHTVWGVTPALHSPLMSVTNAISGLTAVGGLALMGGHLYPSTTSQGLAALAAFISSVNIAGGFLVTQRMLDMFKRPTDPPEYNYLYLLPAGTFVGGYLAALYSGYNIEQIMYLGSGLCCVGALAGLSTQGTARLGNALGMIGVAGGLAATLGVLKPGPELLAQMSGAMALGGTIGLTIAKRIQISDLPQLVAAFHSLVGLAAVLTCIAEYIIEYPHFATDAAANLTKIVAYLGTYIGGVTFSGSLIAYGKLQGLLKSAPLLLPGRHLLNAGLLAASVGGIIPFMVDPSFTTGITCLGSVSALSAVMGVTLTAAIGGADMPVVITVLNSYSGWALCAEGFLLNNNLLTIVGALIGSSGAILSYIMCVAMNRSLANVILGGYGTTSTAGGKPMEISGTHTEINLDNAIDMIREANSIIITPGYGLCAAKAQYPIADLVKMLTEQGKKVRFGIHPVAGRMPGQLNVLLAEAGVPYDIVLEMDEINHDFPDTDLVLVIGANDTVNSAAQEDPNSIIAGMPVLEVWKSKQVIVMKRSLGVGYAAVDNPIFYRPNTAMLLGDAKKTCDALQAKVRESYQK

SEQ ID NO: 3 - DNA, 4232 bp, Homo sapiens
gcgccgcggggcccaagcccgggtctgccagcgcgagctcctctcgcggccctcagggcacagcccaaggctgtcagcctcccggcccagtgatttgccttcaaggaaactggggagtcagaaaattgggaactcatatcaacatggcaaacctattgaaaacagtggtgactggctgctcgtgtcctctacttagcaatttggggtcctgtaagggtctacgtgtgaagaaggattttttacgaacattttatactcaccaagaactgtggtgtaaagcgcctgtaaaaccaggaattccatataagcaactgactgttggagtccccaaagagatattccaaaatgagaagcgagtggcattgtctcctgctggtgttcagaacttggtcaagcagggttttaatgttgtcgtggaatcgggtgcgggcgaagcttccaagttctcagatgatcactatagagtggcaggtgcccaaatccaaggggcaaaggaagtgctggcttctgatttggtggtcaaagtgcgagcccctatggttaatccaacattaggtgttcatgaagctgaccttttaaagacatcaggaacgctgattagttttatttacccagcccaaaatccagagttgctaaataaactttcccaaagaaaaactacagttctggcaatggaccaggttccaagagtcacaattgctcagggatatgatgcgctaagctccatggccaacattgcgggttataaggctgttgtcctagcagcaaatcattttggacgtttttttactggtcagatcacagctgctggaaaagttcctccagctaagattctgatagttggtggtggtgttgctgggcttgcttctgcaggcgccgaaaagtcgatgggtgcaattgttcgaggatttgacacaagagctgccagtttggaacagttcaagtctcttggtgctgagcccttggaggtggacttgaaggaatctggtgagggacaaggaggatatgcaaaagagatgtccaaagagttcattgaagctgaaatgaaactctttgctcaacaatgcaaggaggtagacatccttatcagcacagcacttattccaggtaaaaaagctccagttttatttaataaagaaatgattgagtcaatgaaggaaggttcagttgttgtggatttagctgctgaggctggtggaaactttgaaaccactaagccaggagaactctacattcataagggaattactcacataggctacacagacctgcccagccgaatggccactcaggccagcaccctatattccaacaacatcaccaaactcctgaaggccatcagcccggacaaagataatttttattttgatgtgaaagatgactttgactttggtacgatgggtcatgtcattagaggaactgtagtgatgaaagatggtaaagtgattttcccagctcccacaccgaaaaatattcctcaaggtgccccagtaaaacagaagacagtggctgagctggaagctgaaaaagcagctaccattacacccttcaggaagacaatgtcaacggcttctgcatatacagcaggtctcacagggatactgggtttgggcattgcggctcccaatctagccttttctcagatggtgaccacttttggcttggctggcattgtggggtatcataccgtctggggagtgacccctgctctccactcaccactgatgtctgtgacaaatgcaatctcagggctgactgcagttggtgggttggcactgatgggaggacatttgtatccttccacaacttctcagggccttgctgctcttgctgcattcatatcctctgtcaacattgcaggtggctttctggtgactcagagaatgctggacatgttcaagcgtcccactgaccccccagaatacaactacctgtacctgctccctgccggcacctttgttggtggatatttagctgccctctacagtggttataacattgaacagatcatgtacctaggctcgggtttgtgctgtgtcggtgccttggctggcctctccacccagggaacagcacgtcttggcaatgcactgggcatgattggggttgctggaggactggcagccaccctcggagtcctaaaaccgggcccagaattactagctcagatgtctggagcgatggctttgggtggtaccattggattgacaattgccaaacgcatccagatttctgatttacctcaattagttgctgcttcacacagtttagtgggtttggcagctgtacttacttgcatagctgagtacattatagaatatccacattttgctccggatgcagcagcaaatctcaccaagattgtggcctacctcggcacttacattggtggcgtcacctttagtgggtctctcattgcctatggaaaattgcagggtctcctgaaatctgcccctctcctactgcctggaaggcacttactcaatgcaggcttactggctgctagtgtgggcgggataatcccattcatggtggacccaagctttactactggcatcacctgtctgggttcagtgtctgctctctctgctgtcatgggtgtgactttgacagctgctattgggggtgctgacatgcccgtcgttatcactgtgctgaacagctactcaggctgggccctgtgtgcagagggcttcctgctcaacaacaatctgctgaccatcgtgggtgcactcataggctcgtctggtgctcccctgtcatacatcatgtgtgtggcaatgaatcgctccctggctaatgtgattcttggaggctatggcaccacttcaacagctggtggaaaacccatggaaatttctggcacacatacggaaatcaaccttgacaatgcaattgacatgattcgagaagctaatagcattatttttacaccaggctatggtctctgtgcagccaaagctcaataccccattgctgatttggtaaagatgctcactgagcaaggcaaaaaagtcaggtttggaattcacccagttgcaggccgaatgcctggtcagcttaatgtgctgctggctgaggctggtgtgccatatgacattgtgttggaaatggatgagatcaaccatgattttccagatactgatttggtccttgtaattggagctaatgacactgttaattcagcagctcaagaagatcccaactctattattgcaggcatgccagtccttgaggtctggaaatcaaagcaggtgattgttatgaagaggtctttgggtgttggctatgctgcagtggacaatccaatcttctacaaacctaacacggccatgcttctaggtgatgccaagaaaacatgtgacgcgctccaggcgaaagttagagaatcctatcagaagtaaatattaaggatcaagctgttagctaatatgccacctctgcagttttgggaacaggcaaataaagtatcagtatacatggtgatgtacatctgtagcaaagctcttggagcaaaatgaagactgaagaaagcaaagcaaaaactgtatagagagatttttcaaaagcagtaatccctcaattttaaaaaaggattgaaaattctaaatgtctttctgtgcatattttttgtgttaggaatcaaaagtattttataaaaggagaaagaacagcctcattttagatgtagtcctgttggattttttatgcctcctcagtaaccagaaatgttttaaaaaactaagtgtttaggatttcaagacaacattatacatggctctgaaatatctgacacaatgtaaacattgcaggcacctgcattttatgttttttttttcaacaaatgtgactaatttgaaacttttatgaacttctgagctgtccccttgcaattcaaccgcagtttgaattaatcatatcaaatcagttttaattttttaaattgtacttcagagtctatatttcaagggcacattttctcactactattttaatacattaaaggactaaataatctttcagagatgctggaaacaaatcacttgctttatatgtttcattagaataccaatgaaacatacaacttgaaaattagtaatagtattttggaagatcccatttctaattggagatctctttaatttcgatcaacttataatgtgtagtactatattaagtgcacttgagtgggattcaacatttgactaataaaatgagttcatcatgttggc

SEQ ID NO: 4 - protein, 1086 aa, Homo sapiens
MANLLKTVVTGCSCPLLSNLGSCKGLRVKKDFLRTFYTHQELWCKAPVKPGIPYKQLTVGVPKEIFQNEKRVALSPAGVQNLVKQGFNVVVESGAGEASKFSDDHYRVAGAQIQGAKEVLASDLVVKVRAPMVNPTLGVHEADLLKTSGTLISFIYPAQNPELLNKLSQRKTTVLAMDQVPRVTIAQGYDALSSMANIAGYKAVVLAANHFGRFFTGQITAAGKVPPAKILIVGGGVAGLASAGAEKSMGAIVRGFDTRAASLEQFKSLGAEPLEVDLKESGEGQGGYAKEMSKEFIEAEMKLFAQQCKEVDILISTALIPGKKAPVLFNKEMIESMKEGSVVVDLAAEAGGNFETTKPGELYIHKGITHIGYTDLPSRMATQASTLYSNNITKLLKAISPDKDNFYFDVKDDFDFGTMGHVIRGTVVMKDGKVIFPAPTPKNIPQGAPVKQKTVAELEAEKAATITPFRKTMSTASAYTAGLTGILGLGIAAPNLAFSQMVTTFGLAGIVGYHTVWGVTPALHSPLMSVTNAISGLTAVGGLALMGGHLYPSTTSQGLAALAAFISSVNIAGGFLVTQRMLDMFKRPTDPPEYNYLYLLPAGTFVGGYLAALYSGYNIEQIMYLGSGLCCVGALAGLSTQGTARLGNALGMIGVAGGLAATLGVLKPGPELLAQMSGAMALGGTIGLTIAKRIQISDLPQLVAASHSLVGLAAVLTCIAEYIIEYPHFAPDAAANLTKIVAYLGTYIGGVTFSGSLIAYGKLQGLLKSAPLLLPGRHLLNAGLLAASVGGIIPFMVDPSFTTGITCLGSVSALSAVMGVTLTAAIGGADMPVVITVLNSYSGWALCAEGFLLNNNLLTIVGALIGSSGAPLSYIMCVAMNRSLANVILGGYGTTSTAGGKPMEISGTHTEINLDNAIDMIREANSIIFTPGYGLCAAKAQYPIADLVKMLTEQGKKVRFGIHPVAGRMPGQLNVLLAEAGVPYDIVLEMDEINHDFPDTDLVLVIGANDTVNSAAQEDPNSIIAGMPVLEVWKSKQVIVMKRSLGVGYAAVDNPIFYKPNTAMLLGDAKKTCDALQAKVRESYQK

SEQ ID NO: 5 - DNA, 21 bp, Artificial sequence
  Synthetic primer
cctgcgacttcaacagcaact

SEQ ID NO: 6 - DNA, 21 bp, Artificial sequence
  Synthetic primer
ccaggaaatgagcttgacaaa

SEQ ID NO: 7 - DNA, 20 bp, Artificial sequence
  Synthetic primer
gatagttggtggtggcgttg

SEQ ID NO: 8 - DNA, 20 bp, Artificial sequence
  Synthetic primer
gatgtccacctccttgcact

SEQ ID NO: 9 - DNA, 20 bp, Artificial sequence
  Synthetic primer
gtgcattgaaccctcaaagg

SEQ ID NO: 10 - DNA, 22 bp, Artificial sequence
  Synthetic primer
caggtaagaaagctcctgtttt

SEQ ID NO: 11 - DNA, 20 bp, Artificial sequence
  Synthetic primer
tcctgctattcctcctcctg

SEQ ID NO: 12 - DNA, 21 bp, Artificial sequence
  Synthetic primer
gctgccttgactttggatatt

SEQ ID NO: 13 - DNA, 20 bp, Artificial sequence
  Synthetic primer
cagcacagctctgattccag

SEQ ID NO: 14 - DNA, 24 bp, Artificial sequence
  Synthetic primer
gtcaaaccgaagaccgtggctgag

SEQ ID NO: 15 - DNA, 21 bp, Artificial sequence
  Synthetic primer
gtgatgaaggatggcaaagtg

SEQ ID NO: 16 - DNA, 22 bp, Artificial sequence
  Synthetic primer
gcaggtggttttctggtgactc